(19) United States
(12) Patent Application Publication
(10) Pub. No.: US 2026/0008801 A1
(43) Pub. Date: Jan. 8, 2026

(54) BIO-BASED SURFACTANTS DERIVED FROM CARBOHYDRATES

(71) Applicants: COÖPERATIE KONINKLIJKE COSUN U.A., Breda (NL); STICHTING RADBOUD UNIVERSITEIT, Nijmegen (NL)

(72) Inventors: Adeline RANOUX, Dinteloord (NL); Henricus Wilhelmus Carolina RAAIJMAKERS, Roosendaal (NL); Thomas Jan BOLTJE, Doorn (NL); Laura JANSEN, Hoeven (NL)

(73) Assignees: COÖPERATIE KONINKLIJKE COSUN U.A., Breda (NL); STICHTING RADBOUD UNIVERSITEIT, Nijmegen (NL)

(21) Appl. No.: 18/871,520

(22) PCT Filed: Jun. 16, 2023
(86) PCT No.: PCT/EP2023/066313

§ 371 Date: Dec. 4, 2024

(30) Foreign Application Priority Data
Jun. 16, 2022 (EP) 22179458.9

(51) Int. Cl. C07H 5/04; B01J 23/52; C07H 1/00

(52) U.S. Cl. CPC C07H 5/04; B01J 23/52; C07H 1/00

(57) ABSTRACT

The present invention relates to new carbohydrate derivatives. The derivatives can be prepared via a method that involves direct amination. The derivatives can be used for various applications, for instance as surfactants.

### FIELD OF THE INVENTION

The present invention relates to new carbohydrate derivatives. The derivatives can be prepared via a method that involves direct amination. The derivatives can be used for various applications, for instance as surfactants.

### BACKGROUND

The use of biomass as a source of chemical products is an attractive proposition since it provides a sustainable alternative for chemicals derived from fossil sources. Biomass sources that stem from food production are especially useful since the biomass can be grown without affecting food production. This is important since the world population is rising, whilst the arable land surface area is decreasing. In this respect, the use of sugar beet pulp (SBP) produced during the processing of sugar beets as a source of biomass is very attractive. The use of biomass feed stocks may lead to more readily biodegradable chemical products. Notable in this regard is the development of synthetic routes towards biodegradable glucose-derived surfactants. WO92/06070 and WO92/06072 describe processes for manufacturing a linear glucamide surfactant comprising reacting an N-alkylglucamine, a fatty ester and a catalyst.

The main oligosaccharide constituents of sugar beet pulp are hemicellulose, cellulose, and pectin. Hemicelluloses and pectins can be further processed to afford monosaccharides such as L-Arabinose (L-Ara) and D-Galacturonic acid (D-GalA), respectively.

Surfactants are an important class of molecules as they play an important role in the home and personal care industry, amongst others. Currently used surfactants are often derived from petrochemicals and can be damaging for the human skin and harmful to the environment. Anionic products such as sodium lauryl sulfate or sodium lauryl ether sulfate are especially used for their higher performance compared to non-ionic derivatives but are also known to be sensitizers and possibly irritating. In contrast, carbohydrate based surfactants from renewable raw materials may offer many advantages as they are generally biodegradable, non-toxic for humans, and odorless. In addition, these surfactants are expected to be mild to the skin, which is advantageous in cosmetic applications (Lourith, N. & Kanlayavattanakul, M. Natural surfactants usedin cosmetics: glycolipids. International journal of cosmetic science 31, 255-261 (2009).)

Industrial chemical processes can often depend on chemicals that are hazardous to the environment, or can lead to side products that have to be considered ecotoxic. There is an ongoing need for new synthetic methods that have an improved ecotoxicity profile. Such processes are often referred to as ‘green’. Concurrently there is a need for alternative products that can be manufactured via such green processes.

### SUMMARY OF THE INVENTION

There is a need for new (non-ionic or ionic) surfactants with useful properties. There is a need for new bio-based surfactants. There is a need for new green surfactants. There is a need for new surfactants that can be biodegradable or non-irritating. There is a need for new green methods that can yield useful substances. There is a need for new green methods that can valorize waste streams. There is a need for new methods that can yield bio-based surfactants.

The inventors have identified arabinose (Ara) and galacturonic acid (GalA) as offering opportunities for the development of new bio-based products. Due to their hydrophilic nature and renewable resource, these molecules were found to be ideal to serve as the polar head groups for the development of new sugar- and bio-based surfactants. The present disclosure reports the synthesis and characterisation of non-ionic, anionic, cationic and amphoteric surfactants using the monosaccharides GalA (such as D-GalA) and Ara (such as L-Ara). Oxidative and reductive amination reactions are applied varying with several primary, secondary, linear and branched alkyl amines, resulting in surfactants containing amide and amine linkages.

Accordingly, the invention provides a compound of general formula (0):

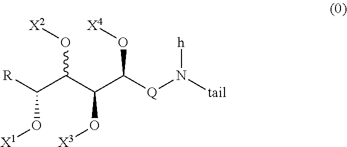

wherein R is —H, —CH₂—Rr, or —C(═O)—Rr; Rr is —OH, —Oh¹, —NH₂, —NH(h¹), or —Nh¹h², wherein h¹ and h² are independently a linear, branched, or cyclic C₁₋₆ alkyl, alkenyl, or alkynyl moiety wherein each carbon atom is optionally substituted by halogen, alkoxy, or haloalkoxy; X¹, X², X³, and X⁴ are in each instance independently hydrogen or a linear, branched, or cyclic C₁₋₆ acyl moiety wherein each carbon atom is optionally substituted by halogen, alkoxy, or haloalkoxy, and wherein the acyl chain is optionally unsaturated, wherein two instances of X¹, X², X³, and X⁴ can together form a bridging moiety to form a five- or six-membered ring; Q is —CH₂— or —C(═O)—; h is a hydrogen or a linear, branched, or cyclic C₁₋₂₀ alkyl, alkenyl, or alkynyl moiety wherein each carbon atom is optionally substituted by halogen, alkoxy, or haloalkoxy; or wherein h is an independently selected instance of tail; tail is a linear, branched, or cyclic C₁₋₄₀ alkyl, alkenyl, or alkynyl moiety, wherein each carbon atom is optionally substituted by one or more halogen, -h^{t}, —O-h^{t}, —C(═O)Oh^{t}, —C(═O)N(h^{t})₂, —OC(═O)-h^{t}, —N(h^{t})C(═O)-h^{t}, ═O, or —N(h^{t})₂, wherein each instance of h^{t} is independently hydrogen or a linear, branched, or cyclic C₁₋₂₀ alkyl, alkenyl, or alkynyl moiety wherein each carbon atom of h^{t} is optionally substituted by halogen, alkoxy, or haloalkoxy; or wherein tail is of general formula —(CH₂CH₂O)₁₋₄₅(CH₂)₀₋₂H; or a salt or N-oxide thereof. This compound is preferably of general formula (I), and in some embodiments it is of general formula (II):

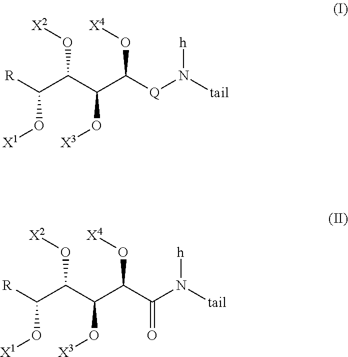

In some embodiments the compound is of general formula (III-og), (III-oa), (III-ra), or (III-ra):

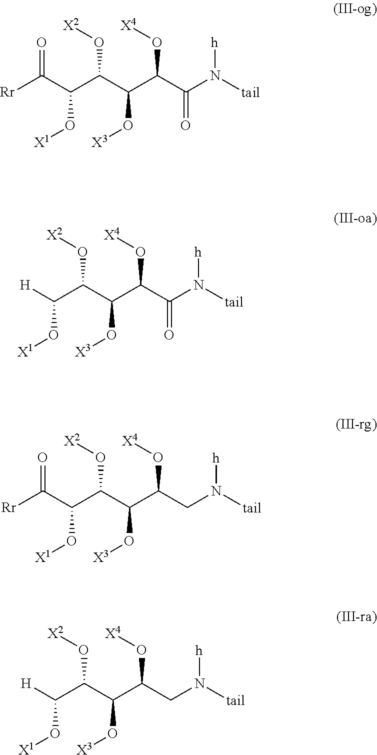

Preferably, Rr is —OH or —Oh¹, preferably —OH; h¹ is —CH₃, —CH₂CH₃, —CH(CH₃)₂, or —C(CH₃)₃; X¹, X², X³, and X⁴ are in each instance independently hydrogen or —C(═O)CH₃, and optionally each of X¹, X², X³, and X⁴ represent the same moiety, more preferably each of X¹, X², X³, and X⁴ represent hydrogen; h is hydrogen or a linear, branched, or cyclic C₁₋₂₀ alkyl, alkenyl, or alkynyl moiety, preferably hydrogen, —CH₃, or —CH₂CH₃, more preferably hydrogen or —CH₃; or tail is a linear or branched C₁₋₂₂ alkyl, alkenyl, or alkynyl moiety, wherein up to two carbon atoms are optionally substituted by halogen, -h^{t}, —O-h^{t}, —C(═O)Oh^{t}, C(═O)N(h^{t})₂, —OC(═O)-h^{t}, —N(h^{t})C(═O)-h^{t}, ═O, or —N(h^{t})₂, wherein each instance of h^{t} is independently hydrogen or a linear or branched C₁₋₁₄ alkyl, alkenyl, or alkynyl moiety.

Preferably tail is a linear C₃₋₂₀ alkyl, more preferably wherein tail is —(CH₂)₃—CH₃, —(CH₂)₅—CH₃, —(CH₂)₇—CH₃, —(CH₂)₉—CH₃, —(CH₂)₁₁—CH₃, —CH₂—CH(CH₃)—CH₃, —(CH₂)₁₀—COOH, —CH(CH₃)—(CH₂)₅—CH₃, —CH(CH₃)—(CH₂)₆—CH₃, —CH₂—CH(CH₂CH₃)—(CH₂)₃—CH₃, —(CH₂CH₂O)₃—CH₂CH₃, or —(CH₂CH₂O)₃—CH₃. In some embodiments h is a linear, branched, or cyclic C₁₋₂₀ alkyl, alkenyl, or alkynyl moiety, preferably —CH₃, or —CH₂CH₃, more preferably —CH₃. The compound can be of general formula (IV) or (V):

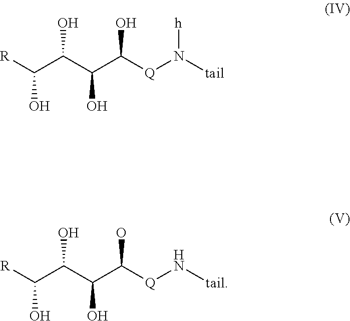

Also provided is a composition comprising a compound as defined above and a solvent or excipient.

Also provided is a method for producing an amide-derivative of a saccharide, comprising the steps of: i) providing a saccharide; ii) providing a primary or secondary amine, preferably of general formula tail-NH-h wherein tail and h are as defined above; iii) reacting the saccharide and the amine in the presence of a metal catalyst at a temperature of at most 60° C. to yield the amide-derivative; iv) optionally isolating the amide-derivative.

Preferably the saccharide is derived from biomass, preferably from agrowaste, more preferably from vegetal pulp such as sugar beet pulp, and/or wherein the saccharide is a monosaccharide, preferably arabinose or galacturonic acid, more preferably L-arabinose or D-galacturonic acid, most preferably D-galacturonic acid. Preferably the amine of step ii) is of general formula tail-NH₂, wherein tail is a linear C₃₋₂₀ alkyl. Preferably the metal catalyst is a gold catalyst, which is preferably a gold chloride, more preferably a gold oxide such as AuTiO₂ or a tetrachloroaurate such as KAuCl₄. The reacting of step iii) is preferably performed in the presence of a base, preferably a non-nucleophilic base, more preferably an inorganic base such as a carbonate salt such as Cs₂CO₃ or K₂CO₃, and wherein the base is preferably present at about 1 to about 300 mol-%, such as at about 200 mol-%.

In some embodiments of the method, a) the reacting of step iii) is performed at a temperature of about 20-60° C., preferably at about 20-40° C., more preferably at about 30-40° C. such as at about 35° C.; b) the metal catalyst is present at about 0.2-15 mol % such as at about 10 mol-%; c) the reacting of step iii) is performed in a protic solvent; d) the reacting of step iii) is performed in a single step; e) the reacting of step iii) is performed without the addition of an oxidant; and/or f) the reacting of step iii) is performed under ambient background radiation.

### DESCRIPTION OF THE INVENTION

The inventors have identified arabinose (Ara) and galacturonic acid (GalA) as offering opportunities for the development of new bio-based products. Due to their hydrophilic nature and renewable resource, these molecules were found to be ideal to serve as the polar head groups for the development of new sugar- and bio-based surfactants. The present disclosure reports the synthesis and characterisation of non-ionic, anionic, cationic and amphoteric surfactants using the monosaccharides GalA (such as D-GalA) and Ara (such as L-Ara). Oxidative and reductive amination reactions are applied varying with several primary, secondary, linear and branched alkyl amines, resulting in surfactants containing amide and amine linkages.

### Compounds

Thus the invention provides a compound of general formula (0):

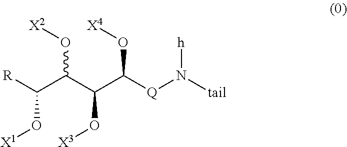

wherein
R is —H, —CH₂—Rr, or —C(═O)—Rr;
Rr is —OH, —Oh¹, —NH₂, —NH(h¹), or —Nh¹h², wherein h¹ and h² are independently a linear, branched, or cyclic C₁₋₆ alkyl, alkenyl, or alkynyl moiety wherein each carbon atom is optionally substituted by halogen, alkoxy, or haloalkoxy;
X¹, X², X³, and X⁴ are in each instance independently hydrogen or a linear, branched, or cyclic C₁₋₆ acyl moiety wherein each carbon atom is optionally substituted by halogen, alkoxy, or haloalkoxy, and wherein the acyl chain is optionally unsaturated, wherein two instances of X¹, X², X³, and X⁴ can together form a bridging moiety to form a five- or six-membered ring;
Q is —CH₂— or —C(═O)—;
h is a hydrogen or a linear, branched, or cyclic C₁₋₂₀ alkyl, alkenyl, or alkynyl moiety wherein each carbon atom is optionally substituted by halogen, alkoxy, or haloalkoxy;
   or wherein h is an independently selected instance of tail;
tail is a linear, branched, or cyclic C₁₋₄₀ alkyl, alkenyl, or alkynyl moiety, wherein each carbon atom is optionally substituted by one or more halogen, -h^{t}, —O-h^{t}, —C(═O)Oh^{t}, —C(═O)N(h^{t})₂, —OC(═O)-h^{t}, —N(h^{t})C(═O)-h^{t}, ═O, or —N(h^{t})₂, wherein each instance of h^{t} is independently hydrogen or a linear, branched, or cyclic C₁₋₂₀ alkyl, alkenyl, or alkynyl moiety wherein each carbon atom of h^{t} is optionally substituted by halogen, alkoxy, or haloalkoxy;
   or wherein tail is of general formula —(CH₂CH₂O)₁₋₄₅(CH₂)₀₋₂H;
or a salt or N-oxide thereof. Such a compound is referred to hereinafter as a compound according to the invention, or “the compound” as will be clear from context. When a compound is an N-oxide, preferably h is a linear, branched, or cyclic C₁₋₂₀ alkyl, alkenyl, or alkynyl moiety wherein each carbon atom is optionally substituted by halogen, alkoxy, or haloalkoxy; or an independently selected instance of tail;
Preferably, the compound is of general formula (I):

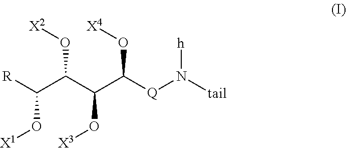

R can be seen as being positioned at the tip of the head of the surfactant. R is —H, —CH₂—Rr, or —C(═O)—Rr. In preferred embodiments R is —H or —C(═O)—Rr. When the compound is derived from Ara, R is preferably —H. When the compound is derived from GalA such as D-GalA, R is preferably —CH₂—Rr or —C(═O)—Rr, more preferably —C(═O)—Rr.

Rr is a group that can be seen as forming a larger moiety together with the adjacent carbonyl moiety, and the larger moiety is generally a carboxylic acid or a derivative thereof. Thus Rr is —OH, —Oh¹, —NH₂, —NH(h¹), or —Nh¹h², wherein h¹ and h² are independently a linear, branched, or cyclic C₁₋₆ alkyl, alkenyl, or alkynyl moiety wherein each carbon atom is optionally substituted by halogen, alkoxy, or haloalkoxy. Preferably Rr is —OH or —Oh¹, more preferably —OH. In some embodiments Rr is —Oh¹, —NH₂, —NH(h¹), or —Nh¹h². In some embodiments Rr is —NH₂, —NH(h¹), or —Nh¹h².

In preferred embodiments h¹ and h² represent the same moiety when both are present. h¹ and h² are preferably linear. h¹ and h² are preferably C₁₋₄, more preferably C₁₋₃, even more preferably C₁₋₂, and most preferably C₁, for instance —CH₃. h¹ and h² are preferably alkyl. When h¹ and h² are optionally substituted, preferred halogens are F and Cl, more preferably F, a preferred alkoxy is C₁₋₄alkoxy, most preferably —O—CH₃, and a preferred haloalkoxy is fluorinated or chlorinated C₁₋₄alkoxy such as —CF₃. In preferred embodiments h¹ and h² are not optionally substituted.

Throughout this disclosure, preferred examples of C₁₋₄alkyl are —CH₃, —CH₂CH₃, —CH(CH₃)₂, —C(CH₃)₃, —CH₂CH₂CH₃, —CH(CH₃)CH₂CH₃, —CH₂CH(CH₃)₂, cyclopropyl, and cyclobutyl, more preferably —CH₃, —CH₂CH₃, —CH(CH₃)₂, —C(CH₃)₃. Preferred examples of C₁₋₃alkyl are —CH₃, —CH₂CH₃, —CH(CH₃)₂, —CH₂CH₂CH₃, and cyclopropyl, more preferably —CH₃, —CH₂CH₃, and —CH(CH₃)₂. Preferred examples of C₁₋₂alkyl are —CH₃ and —CH₂CH₃, more preferably —CH₃. Throughout this description, when C₁₋₆ alkyl, alkenyl, or alkynyl moieties are described, it is to be understood that the described alkenyl or alkynyl moieties are C₂₋₆ moieties, and so forth for C₁₋₅, C₁₋₄, C₁₋₃, and C₁₋₂. Whenever such a moiety is a C₁ moiety, it is an alkyl moiety.

In preferred embodiments R is H or C(═O)—Rr. Such compounds are of general formula (III-og), (III-oa), (III-rg), or (III-ra):

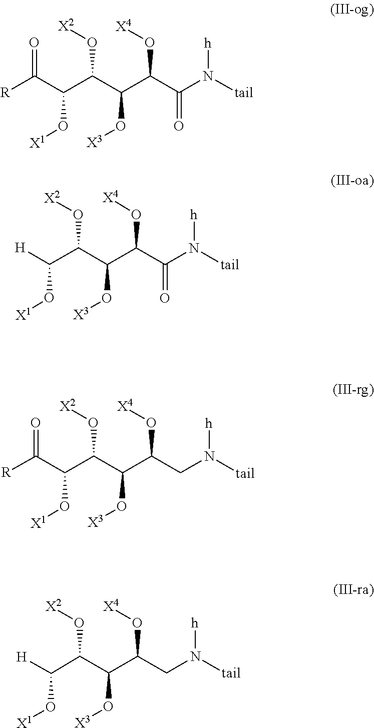

In some embodiments the compounds are of general formula (III-oa), (III-rg), or (III-ra). In some embodiments the compounds are of general formula (III-og), (III-rg), or (III-ra). In some embodiments the compounds are of general formula (III-og), (III-oa), or (III-ra). In some embodiments the compounds are of general formula (III-og), (III-oa), or (III-rg). In highly preferred embodiments the compounds are of general formula (III-og) or (III-oa). In preferred embodiments the compounds are of general formula (III-rg) or (III-ra). In preferred embodiments the compounds are of general formula (III-og) or (III-rg). In preferred embodiments the compounds are of general formula (III-oa) or (III-ra). General formula (III-og) is most preferred.

X¹, X², X³, and X⁴ are in each instance independently hydrogen or a linear, branched, or cyclic C₁₋₇ acyl moiety wherein each carbon atom is optionally substituted by halogen, alkoxy, or haloalkoxy, and wherein the acyl chain is optionally unsaturated, wherein two instances of X¹, X², X³, and X⁴ can together form a bridging moiety to form a five- or six-membered ring. In preferred embodiments, X¹, X², X³, and X⁴ each represent the same moiety. Preferably X¹, X², X³, and X⁴ are in each instance independently hydrogen or —C(═O)CH₃, more preferably each of X¹, X², X³, and X⁴ represent hydrogen.

When not hydrogen, X¹, X², X³, and X⁴ are preferably linear. When not hydrogen, X¹, X², X³, and X⁴ are preferably C₂₋₆, more preferably C₂₋₅, even more preferably C₂₋₄, still more preferably C₂-3, and most preferably C₂, for instance —C(═O)CH₃. When X¹, X², X³, and X⁴ are optionally substituted, preferred halogens are F and C₁, more preferably F, a preferred alkoxy is C₁₋₄alkoxy, most preferably —O—CH₃, and a preferred haloalkoxy is fluorinated or chlorinated C₁₋₄alkoxy such as —CF₃. In preferred embodiments X¹, X², X³, and X⁴ are not optionally substituted.

Throughout this description, preferred examples of C₁₋₅acyl are —C(═O)H, —C(═O)CH₃, —C(═O)CH₂CH₃, —C(═O)CH(CH₃)₂, —C(═O)C(CH₃)₃, —C(═O)CH₂CH₂CH₃, —C(═O)CH(CH₃)CH₂CH₃, —C(═O)CH₂CH(CH₃)₂, —C(═O)-cyclopropyl, and —C(═O)cyclobutyl, more preferably —C(═O)CH₃, —C(═O)CH₂CH₃, —C(═O)CH(CH₃)₂, —C(═O)C(CH₃)₃, or —C(═O)CH₂CH₂CH₃. Preferred examples of C₁₋₄acyl are —C(═O)H, —C(═O)CH₃, —C(═O)CH₂CH₃, —C(═O)CH(CH₃)₂, —C(═O)CH₂CH₂CH₃, and —C(═O)-cyclopropyl, more preferably —C(═O)CH₃, —C(═O)CH₂CH₃, —C(═O)CH(CH₃)₂, or —C(═O)CH₂CH₂CH₃. Preferred examples of C₁₋₃acyl are —C(═O)H, —C(═O)CH₃, and —C(═O)CH₂CH₃, more preferably —C(═O)CH₃, and —C(═O)CH₂CH₃. A most preferred acyl is C₂ acyl, preferably —C(═O)CH₃.

Throughout this disclosure an acyl chain is optionally unsaturated. Preferred unsaturated acyl chains are —C(═O)—CH═CH₂ or —C(═O)—CH₂—CH₂—C≡CH.

When not hydrogen, X¹, X², X³, and X⁴ can be understood to be protecting groups. These groups are not necessarily present in the surfactant when ready for use, but as is understood by a skilled person, the protected compounds are readily converted into the compounds wherein at least some, but preferably all of X¹, X², X³, and X⁴ are hydrogen. Therefore in preferred embodiments X¹, X², X³, and X⁴ are in each instance independently a protecting group selected from acetyl (Ac), benzoyl (Bz), benzyl (Bn), β-methoxyethoxymethyl (MEM), dimethoxytrityl, [bis-(4-methoxyphenyl)phenylmethyl](DMT), methoxymethyl (MOM), methoxytrityl [(4-methoxyphenyl)diphenylmethyl](MMT), p-methoxybenzyl (PMB), p-methoxyphenyl (PMP), methylthiomethyl, pivaloyl (Piv), tetrahydropyranyl (THP), tetrahydrofuran (THF), trityl (triphenylmethyl, Tr), silyls such as trimethylsilyl (TMS), tert-butyldimethylsilyl (TBDMS), tri-iso-propylsilyloxymethyl (TOM) and triisopropylsilyl (TIPS), methyl and ethoxyethyl (EE). More preferably, X¹, X², X³, and X⁴ are in each instance independently acetyl, benzyl or p-methoxybenzyl (PMB). In preferred embodiments X¹, X², X³, and X⁴ are in each instance independently chosen from H, acetyl (Ac), benzyl (Bz), and paramethoxybenzyl (PMB), most preferably H or acetyl. When each of X¹, X², X³, and X⁴ is H, the compound is ready for use as for instance a surfactant. Such compounds are of general formula (IV):

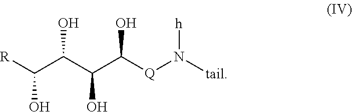

Two instances of X¹, X², X³, and X⁴ can together form a bridging moiety to form a five- or six-membered ring. It is to be understood that all four instances of X¹, X², X³, and X⁴ can together form two instances of such a bridging moiety. Bridging moieties as such are known to a skilled person, with common examples being acetonides or cycloalkylidenes such as cyclopentylidene or cyclohexylidene. Preferred bridging moieties are —CH₂—, —C(CH₃)₂—, spiro-cyclopentane, and spiro-cyclohexane, more preferably —C(CH₃)₂—, spiro-cyclopentane, and spiro-cyclohexane, most preferably —C(CH₃)₂—. Bridging moieties are preferably formed between two adjacent instances of X¹, X², X³, and X⁴. Bridging moieties are preferably not formed between X¹ and X⁴. In preferred embodiments none of X¹, X², X³, and X⁴ together form a bridging moiety.

Q is —CH₂— or —C(═O)—. When Q is —C(═O)— the compounds are conveniently accessible via oxidative amination, for instance via a method according to the invention, and thus in preferred embodiments Q is —C(═O)—. When Q is —CH₂—, the compounds are conveniently accessible via reductive amination, and thus in other preferred embodiments Q is —CH₂—. Compounds wherein Q is —C(═O)— are of general formula (II):

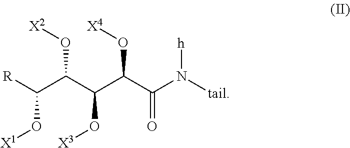

h is a hydrogen or a linear, branched, or cyclic C₁₋₂₀ alkyl, alkenyl, or alkynyl moiety wherein each carbon atom is optionally substituted by halogen, alkoxy, or haloalkoxy; or wherein h is an independently selected instance of tail. In some specific embodiments, h is an independently selected instance of tail. In other embodiments h is hydrogen or a linear, branched, or cyclic C₁₋₂₀ alkyl, alkenyl, or alkynyl moiety, preferably hydrogen, —CH₃, or —CH₂CH₃, more preferably hydrogen or —CH₃. In certain embodiments, h is H. These compounds are sometimes referred to herein as ‘not being N-substituted’. When h is H, the compounds are of general formula (V):

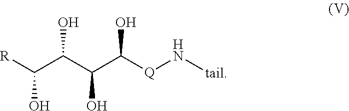

It was found that when h is not H, the compounds had certain improved properties such as improved solubility, improved surfactant properties, and improved foaming behavior. Accordingly in preferred embodiments h is a linear, branched, or cyclic C₁₋₂₀ alkyl, alkenyl, or alkynyl moiety, preferably —CH₃, or —CH₂CH₃, more preferably —CH₃. Here, h is more preferably a linear C₁₋₂₀ alkyl, alkenyl, or alkynyl moiety, preferably —CH₃, or —CH₂CH₃, more preferably —CH₃, even more preferably a linear C₁₋₂₀ alkyl moiety, preferably —CH₃, or —CH₂CH₃, more preferably —CH₃. It was found that for h, compound properties were most improved when C₁₋₂₀ alkyl was C₁₋₆ alkyl, more preferably C₁₋₄ alkyl, even more preferably C₁₋₂ alkyl, most preferably —CH₃.

Solubility is preferably solubility in aqueous solution, more preferably solubility in water, most preferably solubility in demineralised water. A skilled person knows how to assess such solubility. A preferred method for determining solubility is by suspending an excess of the compound in about 2 to 5 ml demineralised water, such as in 4 ml demineralised water, after which the resulting saturated solution is separated from undissolved solids, after which a known volume, such as 1 ml, of the saturated solution is dried to allow weighing of the amount of dissolved compound in the known volume. Preferred compounds according to the invention have a solubility in water of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.2, 4.4, 4.6, 4.8, 5, 5.2, 5.4, 5.6, 5.8, 6, 6.2, 6.4, 6.6, 6.8, 7, 7.5, 8, 8.5, 9, 9.5, 10, 11, 12, 13, 14, 15, 20, 25, 30, 35, 40, 45% or more, wherein the percentage represents the compound's mass fraction of the total solution weight. More preferably the compounds have a solubility in water of at least 0.06%, still more preferably at least 3, 3.1, 3.2, 3.3, 3.4, 3.5, 3.6, 3.7, 3.8, 3.9, 4, 4.2, 4.4, 4.6, 4.8, 5, 5.2, 5.4, 5.6, 5.8, 6, 6.2, 6.4, 6.6, 6.8, 7, 7.5, 8, 8.5, 9, 9.5, 10, 11, 12, 13, 14, 15, 20, 25, 30, 35, 40, 45% or more.

Improved surfactant properties are preferably an improved ability to reduce surface tension. This can be conveniently expressed as −log(concentration to reduce surface tension of water by 20 mN m⁻¹), often denoted as pC₂₀. pC₂₀ values are preferably determined as described in the examples. Preferred compounds according to the invention have a pC₂₀ value of below 61, 51, 50, 45, 40, 35, 34, 33, 32, 31, 30, or lower, more preferably of below 51 or lower, still more preferably of below 31 or lower.

Foaming behaviour is preferably determined as described in the examples. Preferred compounds according to the invention exhibit low foam formation, moderate foam formation, or strong foam formation; preferably moderate foam formation, or strong foam formation; most preferably strong foam formation.

Preferably, when h is not H, properties such as solubility, surfactant properties, and foaming behavior, are improved as compared to the same compound wherein h is H. This improvement is preferably by at least 1%, more preferably at least 5%, still more preferably at least 10%, most preferably at least 15%, and optionally least 20, 25, 30, 35, 40, 45, 50, 55, 60, 65, 70, 75, 80, 85, 90, 95, 100, 110, 120, 130, 140, 150% or more.

In the context of this invention, a halogen is F, Cl, Br, I or At. Preferably, a halogen is F, Cl, Br or I. More preferably, a halogen is C₁ or Br. Most preferably, a halogen is C₁.

In the context of this invention, a C_{X-Y}alkyl, alkenyl, or alkynyl (for instance a C₁₋₃alkyl) is a hydrocarbon wherein the number of C atoms comprised in said hydrocarbon is from X up to Y. A substituted C_{X-Y}hydrocarbon is a substituted hydrocarbon wherein the number of C atoms comprised in the (unsubstituted) hydrocarbon, wherein one or more H atoms have been replaced, is from X up to Y. In other words, a C_{X-Y}hydrocarbon may comprise more than Y C atoms in case said C_{X-Y}hydrocarbon comprises substituent comprising C atoms. All definitions and terms used in the context of substituted hydrocarbons and C_{X-Y}hydrocarbons may be applied mutatis mutandis to specific hydrocarbons such as alkyls, alkenyls, and alkynyls. In this disclosure, unless otherwise indicated, C₁₋₂₀alkyl, alkenyl, and alkynyl are preferably C₁₋₁₈, more preferably C₁₋₁₆, more preferably C₁₋₁₄, more preferably C₁₋₁₂, more preferably C₁₋₁₀, more preferably C₁₋₈, still more preferably C₁₋₆, still more preferably C₁₋₄, most preferably C₁₋₂, and can in preferred embodiments be C₁. Other preferred C₁₋₂₀alkyl, alkenyl, and alkynyl are C₂₋₂₀, more preferably C₄₋₂₀, more preferably C₆₋₂₀, more preferably C₈₋₂₀, more preferably C₁₀₋₂₀, more preferably C₁₂₋₂₀, still more preferably C₁₄₋₂₀, still more preferably C₁₆₋₂₀, optionally C₁₈₋₂₀, and can in some embodiments be C₂₀. Particularly when comprised in h, C₁₋₂₀alkyl, alkenyl, and alkynyl are preferably C₁₋₁₂, more preferably C₁₋₈, more preferably C₁₋₆, more preferably C₁₋₄, even more preferably C₁₋₂, most preferably C₁. Particularly when comprised in tail, C₁₋₄₀alkyl, alkenyl, and alkynyl are preferably C₄₋₂₀, more preferably C₄₋₁₆, more preferably C₄₋₁₄, more preferably C₄₋₁₂, even more preferably C₆₋₁₂, most preferably C₈₋₁₂.

In the context of this invention, an alkyl is a hydrocarbon, wherein each covalent bond comprised in said hydrocarbon is formally a single bond. An alkyl may be linear, branched or cyclic, as understood by the skilled person. In preferred embodiments, alkyl is linear or branched. In highly preferred embodiments alkyl is linear. A branched C_{X-Y}alkyl comprises from x to y carbon atoms in the combination of its longest chain and branch. An example is s-butyl as described below. A cyclic alkyl may be entirely cyclic such as cyclohexyl, but may also comprise a cyclic portion, such as in the C₈-cycloalkyl that is —CH₂-cyclohexyl-para-CH₃.

Examples of C₁₋₅alkyls are methyl (—CH₃), ethyl (—CH₂CH₃), n-propyl (—CH₂CH₂CH₃), i-propyl (—CH(—CH₃)₂), c-propyl, n-butyl (—(CH₂)₃—CH₃), s-butyl (—CH(—CH₃)—CH₂—CH₃), t-butyl (—C(—CH₃)₃), i-butyl (—CH₂—CH(—CH₃)₂), c-butyl, n-pentyl (—(CH₂)₄—CH₃), t-pentyl (—C(—CH₃)₂—CH₂—CH₃), neopentyl (—CH₂—C(—CH₃)₃), isopentyl (—(CH₂)₂—CH(—CH₃)₂), s-pentyl (—CH(—CH₃)—(CH₂)₂—CH₃), 3-pentyl (—CH(—CH₂—CH₃)₂), sec-isopentyl (—CH(—CH₃)—CH(CH₃)₂), active pentyl (—CH₂—CH(—CH₃)—CH₂—CH₃) and c-pentyl. Non-limiting examples of C₁₋₆alkyls are the C₁₋₅alkyls mentioned above, n-hexyl (—(CH₂)₅—CH₃), and c-hexyl. Non-limiting examples of C₁₋₈alkyls are the C₁₋₆alkyls mentioned above, n-heptyl (—(CH₂)₆—CH₃), n-octyl (—(CH₂)₇—CH₃), c-heptyl, and c-hexyl. Non-limiting examples of C₁₋₁₀alkyls are the C₁₋₈alkyls mentioned above, n-nonyl (—(CH₂)₈—CH₃), n-decyl(—(CH₂)₉—CH₃), c-heptyl, and c-hexyl. Non-limiting examples of C₁₋₁₂alkyls are the C₁₋₁₀alkyls mentioned above, n-undecyl (—(CH₂)₁₀—CH₃), and n-dodecyl (—(CH₂)₁₁—CH₃). Non-limiting examples of C₁₋₁₆alkyls are the C₁₋₁₂alkyls mentioned above, n-tetradecyl (—(CH₂)₁₃—CH₃), and n-hexadecyl (—(CH₂)₁₅—CH₃). Preferred alkyl moieties with four or more carbon atoms in the main chain have an even number of such carbon atoms.

In the context of this invention, an unsaturated hydrocarbon is a hydrocarbon, wherein said hydrocarbon comprises a formally double (C═C) or triple C—C bond (C≡C). An alkenyl is an unsaturated alkyl, wherein said unsaturated hydrocarbon comprises a formally double bond, wherein said formally double bond is not part of an aromatic system. Examples of alkenyls are vinyl (—CH═CH₂) and allyl (—CH₂—CH═CH₂). Preferred alkenyl moieties are as alkyl moieties described above, having one or two or three or more double bonds, optionally one or two double bonds, preferably one double bond. In the context of this invention, an alkynyl is an unsaturated hydrocarbon, wherein said unsaturated hydrocarbon comprises a formally triple bond, wherein said formally triple bond is not part of an aromatic system. An example of an alkynyls is ethynyl (—C≡CH). Examples of alkynyls are as alkyl moieties described above, having one or two or three or more triple bonds, optionally one or two triple bonds, preferably one triple bond. A cyclic unsaturated alkyl may be an aryl. In the context of this invention, an aryl is an unsaturated hydrocarbon, wherein said unsaturated hydrocarbon comprises an aromatic system. Examples of aryls are phenyl and benzyl.

In the context of this application, the substitution of a C atom with an alkoxy means replacement of a H atom covalently attached to said C atom, as outlined above, by an —O—R″ group, wherein R″ is an alkyl group. A preferred alkoxy is methoxy (—O—CH₃). In the context of this application, the substitution of a C atom with an haloalkoxy means replacement of a H atom covalently attached to said C atom, as outlined above, by an —O—R″ group, wherein R″ is a substituted alkyl group, wherein the substitution of said alkyl group is by one or more halogens. A preferred haloalkoxy is trifluoromethoxy (—O—CF₃).

In some embodiments there are no optional substitutions. In some embodiments the optional substitutions are halogen or haloalkoxy. In some embodiments the optional substitutions are halogen or alkoxy. In some embodiments the optional substitutions are alkoxy or haloalkoxy. In some embodiments the optional substitutions are halogen. In some embodiments the optional substitutions are haloalkoxy. In some embodiments the optional substitutions are alkoxy.

tail can be regarded as the tail portion of a surfactant, and is a linear, branched, or cyclic C₁₋₄₀ alkyl, alkenyl, or alkynyl moiety, wherein each carbon atom is optionally substituted by one or more halogen, -h^{t}, —O-h^{t}, —C(═O)Oh^{t}, —C(═O)N(h^{t})₂, —OC(═O)-h^{t}, —N(h^{t})C(═O)-h^{t}, ═O, or —N(h^{t})₂, wherein each instance of h^{t} is independently hydrogen or a linear, branched, or cyclic C₁₋₂₀ alkyl, alkenyl, or alkynyl moiety wherein each carbon atom of h^{t} is optionally substituted by halogen, alkoxy, or haloalkoxy; or wherein tail is of general formula —(CH₂CH₂O)₁₋₄₅(CH₂)₀₋₂H.

When tail is a linear, branched, or cyclic C₁₋₄₀ alkyl, alkenyl, or alkynyl moiety, it is preferably linear or branched, more preferably linear. It is preferably alkyl or alkenyl, more preferably alkyl. It is preferably C₁₋₃₀, more preferably C₁₋₂₀, or as described elsewhere herein for C_{X-Y}. tail can also be of general formula —(CH₂CH₂O)₁₋₄₅(CH₂)₀₋₂H, wherein particular examples are —(CH₂CH₂O)₂₋₂₀(CH₂)₀₋₂H, preferably —(CH₂CH₂O)₂₋₆(CH₂)₀₋₂H, more preferably —(CH₂CH₂O)₂₋₄(CH₂)₀₋₂H, such as —(CH₂CH₂O)₃(CH₂)₂H. Preferably tail is a linear C₃₋₂₀ alkyl, more preferably C₄₋₂₀, more preferably C₆₋₁₆, more preferably C₆₋₁₄, even more preferably C₆₋₁₂, still more preferably C₈₋₁₂. Compounds having tail C₈₋₂₀, preferably C₈₋₁₀, showed particularly good foaming behaviour.

In preferred embodiments tail is —(CH₂)₃—CH₃, —(CH₂)₅—CH₃, —(CH₂)₇—CH₃, —(CH₂)₉—CH₃, —(CH₂)₁₁—CH₃, —CH₂—CH(CH₃)—CH₃, —(CH₂)₁₀—COOH, —CH(CH₃)—(CH₂)₅—CH₃, —CH(CH₃)—(CH₂)₆—CH₃, —CH₂—CH(CH₂CH₃)—(CH₂)₃—CH₃, —(CH₂CH₂O)₃—CH₂CH₃, or —(CH₂CH₂O)₃—CH₃.

In tail, each carbon atom is optionally substituted by one or more halogen, -h^{t}, —O-h^{t}, —C(═O)Oh^{t}, —C(═O)N(h^{t})₂, —OC(═O)-h^{t}, —N(h^{t})C(═O)-h^{t}, ═O, or —N(h^{t})₂, wherein each instance of h^{t} is independently hydrogen or a linear, branched, or cyclic C₁₋₂₀ alkyl, alkenyl, or alkynyl moiety wherein each carbon atom of h^{t} is optionally substituted by halogen, alkoxy, or haloalkoxy. In preferred embodiments h^{t} is not further substituted. Preferably h^{t} is —H or —C₁₋₂alkyl. Examples of substituted h^{t} are —CH₂—OH and —CH₂—CH₂—OH.

Preferably tail is a linear or branched C₁₋₂₂ alkyl, alkenyl, or alkynyl moiety, wherein up to two carbon atoms are optionally substituted by halogen, -h^{t}, —O-h^{t}, —C(═O)Oh^{t}, C(═O)N(h^{t})₂, —OC(═O)-h^{t}, —N(h^{t})C(═O)-h^{t}, ═O, or —N(h^{t})₂, wherein each instance of h^{t} is independently hydrogen or a linear or branched C₁₋₁₄ alkyl, alkenyl, or alkynyl moiety.

In some embodiments there are no optional substitutions in tail. In some embodiments the optional substitutions are halogen, -h^{t}, —O-h^{t}, —C(═O)Oh^{t}, —OC(═O)-h^{t}, or ═O. In some embodiments the optional substitutions are halogen, -h^{t}, —O-h^{t}, or ═O. In some embodiments the optional substitutions are halogen, -h^{t}, or ═O, preferably halogen or ═O, optionally halogen or -h^{t}, optionally -h^{t} or ═O.

In preferred compounds, Rr is —OH or —Oh¹, preferably —OH;
h¹ is —CH₃, —CH₂CH₃, —CH(CH₃)₂, or —C(CH₃)₃;
X¹, X², X³, and X⁴ are in each instance independently hydrogen or —C(═O)CH₃, and optionally each of X¹, X², X³, and X⁴ represent the same moiety, more preferably each of X¹, X², X³, and X⁴ represent hydrogen;
h is hydrogen or a linear, branched, or cyclic C₁₋₂₀ alkyl, alkenyl, or alkynyl moiety, preferably hydrogen, —CH₃, or —CH₂CH₃, more preferably hydrogen or —CH₃;
tail is a linear or branched C₁₋₂₂ alkyl, alkenyl, or alkynyl moiety, wherein up to two carbon atoms are optionally substituted by halogen, -h^{t}, —O-h^{t}, —C(═O)Oh^{t}, C(═O)N(h^{t})₂, —OC(═O)-h^{t}, —N(h^{t})C(═O)-h^{t}, ═O, or —N(h^{t})₂, wherein each instance of h^{t} is independently hydrogen or a linear or branched C₁₋₁₄ alkyl, alkenyl, or alkynyl moiety.

In preferred embodiments, the compound is of general formula (IV) or (V).

Preferred compounds according to the invention are N-methyl-N-octyl-L-arabinonamide, N-methyl-N-octyl-D-galactaric acid amide, N-methyl-N-hexyl-L-arabinamine, N-methyl-N-octyl-L-arabinamine, N-methyl-N-dodecyl-L-arabinamine, N-methyl-N-hexyl-D-galacturonic acid amine, N-methyl-N-octyl-D-galacturonic acid amine, N-methyl-N-dodecyl-D-galacturonic acid amine, N,N-dioctyl-D-galacturonic acid amine, N-butyl-L-arabinonamide, N-hexyl-L-arabinonamide, N-octyl-L-arabinonamide, N-Decyl-L-arabinonamide, N-isobutyl-L-arabinonamide, N-(decanoic acid)-L-arabinonamide, N-butyl-D-galactaric acid amide, N-hexyl-D-galactaric acid amide, N-octyl-D-galactaric acid amide, N-butyl-L-arabinamine, N-hexyl-L-arabinamine, N-octyl-L-arabinamine, N-(decanoic acid)-L-arabinamine, N-octyl-D-galacturonic acid amine, N-dodecyl-D-galacturonic acid amine, N-(1-methyloctyl)-D-galacturonic acid amine, N-hexyl-D-galacturonic acid amine, N-methyl-N-hexyl-D-galacturonic acid amine oxide, N-methyl-N-octyl-D-galacturonic acid amine oxide, and N-methyl-N-dodecyl-D-galacturonic acid amine oxide, preferably N-methyl-N-octyl-L-arabinonamide, N-methyl-N-octyl-D-galactaric acid amide, N-methyl-N-hexyl-L-arabinamine, N-methyl-N-octyl-L-arabinamine, N-methyl-N-dodecyl-L-arabinamine, N-methyl-N-hexyl-D-galacturonic acid amine, N-methyl-N-octyl-D-galacturonic acid amine, N-methyl-N-dodecyl-D-galacturonic acid amine, N,N-dioctyl-D-galacturonic acid amine, N-butyl-L-arabinonamide, N-hexyl-L-arabinonamide, N-octyl-L-arabinonamide, N-Decyl-L-arabinonamide, N-isobutyl-L-arabinonamide, N-(decanoic acid)-L-arabinonamide, N-butyl-D-galactaric acid amide, N-hexyl-D-galactaric acid amide, N-octyl-D-galactaric acid amide, N-methylhexyl-L-arabinonamine oxide, N-methyloctyl-L-arabinonamine oxide, N-methyldecyl-L-arabinonamine oxide, N-methyl-N-hexyl-D-galacturonic acid amine oxide, N-ethyl-N-hexyl-D-galacturonic acid amine oxide, N-methyl-N-octyl-D-galacturonic acid amine oxide, N-methyl-N-octyl-D-galacturonic acid amine oxide, N-methyl-N-dodecyl-D-galacturonic acid amine oxide, 1-(N-methyl-N-octyl)-D-glucosamine oxide, 1-(N-methyl-N-decyl)-D-glucosamine oxide, 1-(N-methyl-N-dodecyl)-D-glucosamine oxide, and 1-(N-methyl-N-hexyl)-D-glucosamine oxide.

Preferred compounds wherein h is H are N-butyl-L-arabinonamide, N-hexyl-L-arabinonamide, N-octyl-L-arabinonamide, N-Decyl-L-arabinonamide, N-isobutyl-L-arabinonamide, N-(decanoic acid)-L-arabinonamide, N-butyl-D-galactaric acid amide, N-hexyl-D-galactaric acid amide, N-octyl-D-galactaric acid amide, N-butyl-L-arabinamine, N-hexyl-L-arabinamine, N-octyl-L-arabinamine, N-(decanoic acid)-L-arabinamine, N-octyl-D-galacturonic acid amine, N-dodecyl-D-galacturonic acid amine, N-(1-methyloctyl)-D-galacturonic acid amine, and N-hexyl-D-galacturonic acid amine, more preferably N-butyl-L-arabinonamide, N-hexyl-L-arabinonamide, N-octyl-L-arabinonamide, N-decyl-L-arabinonamide, N-isobutyl-L-arabinonamide, N-(decanoic acid)-L-arabinonamide, N-butyl-D-galactaric acid amide, N-hexyl-D-galactaric acid amide, and N-octyl-D-galactaric acid amide.

Preferred compounds wherein h is not H are N-methyl-N-octyl-L-arabinonamide, N-methyl-N-octyl-D-galactaric acid amide, N-methyl-N-hexyl-L-arabinamine, N-methyl-N-octyl-L-arabinamine, N-methyl-N-dodecyl-L-arabinamine, N-methyl-N-hexyl-D-galacturonic acid amine, N-methyl-N-octyl-D-galacturonic acid amine, N-methyl-N-dodecyl-D-galacturonic acid amine, and N,N-dioctyl-D-galacturonic acid amine.

Preferred compounds wherein Q is —C(═O)— are N-butyl-L-arabinonamide, N-hexyl-L-arabinonamide, N-octyl-L-arabinonamide, N-Decyl-L-arabinonamide, N-isobutyl-L-arabinonamide, N-(decanoic acid)-L-arabinonamide, N-butyl-D-galactaric acid amide, N-hexyl-D-galactaric acid amide, N-octyl-D-galactaric acid amide, N-methyl-N-octyl-L-arabinonamide, and N-methyl-N-octyl-D-galactaric acid amide.

Compounds according to the invention can be salts or N-oxides. A salt can be a pharmaceutically acceptable salt, although for instance cleaning applications do not require this. A salt is preferably a base addition salt. Examples of suitable salts are non-metallic salts such as ammonia salts, and metallic salts such as sodium salts and potassium salts. A skilled person can select suitable salt forms, and their means of production are well known (see e.g. “Occurrence of pharmaceutically acceptable anions and cations in the Cambridge Structural Database” Haynes et al., DOI: 10.1002/jps.20441). A salt can also be an acid addition salt. Acid addition salts are known in the art and examples are HCl salts and acetic acid salts.

N-oxides are known to a skilled person, and are preferably oxides of the nitrogen atom that is linked to Q, tail, and h. A general formula representing an N-oxide is general formula (I-oxide):

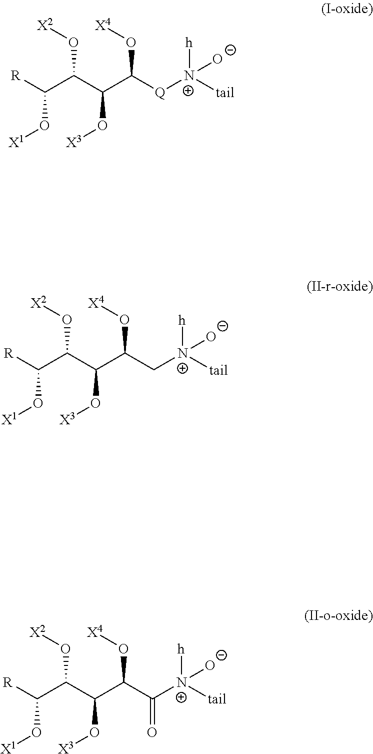

These N-oxide compounds generally have amphiphilic properties at neutral pH and known N-oxides are found in many applications like cleaning agents and cosmetics. They are generally considered soft surfactants. They have a zwitterionic property at every pH, and can for example be formed out of corresponding compounds by treatment with an aqueous hydrogen peroxide solution, preferably under ambient conditions. In preferred embodiments, the N-oxide is of general formula (I-oxide) wherein Q is —CH₂—, which are of general formula (II-r-oxide), or wherein Q is —C(═O)—, which are of general formula (II-o-oxide). Of particular interest are compounds of general formulas (III-rg-oxide) and (III-ra-oxide). Other relevant compounds are compounds of general formulas (III-og-oxide) and (III-oa-oxide). Preferred N-oxides are N-methylhexyl-L-arabinonamine oxide, N-methyloctyl-L-arabinonamine oxide, N-methyldecyl-L-arabinonamine oxide, N-methyl-N-hexyl-D-galacturonic acid amine oxide, N-ethyl-N-hexyl-D-galacturonic acid amine oxide, N-methyl-N-octyl-D-galacturonic acid amine oxide, N-methyl-N-octyl-D-galacturonic acid amine oxide, N-methyl-N-dodecyl-D-galacturonic acid amine oxide, 1-(N-methyl-N-octyl)-D-glucosamine oxide, 1-(N-methyl-N-decyl)-D-glucosamine oxide, 1-(N-methyl-N-dodecyl)-D-glucosamine oxide, or 1-(N-methyl-N-hexyl)-D-glucosamine oxide. More preferred are N-methyl-N-hexyl-D-galacturonic acid amine oxide, N-methyl-N-octyl-D-galacturonic acid amine oxide, and N-methyl-N-dodecyl-D-galacturonic acid amine oxide.

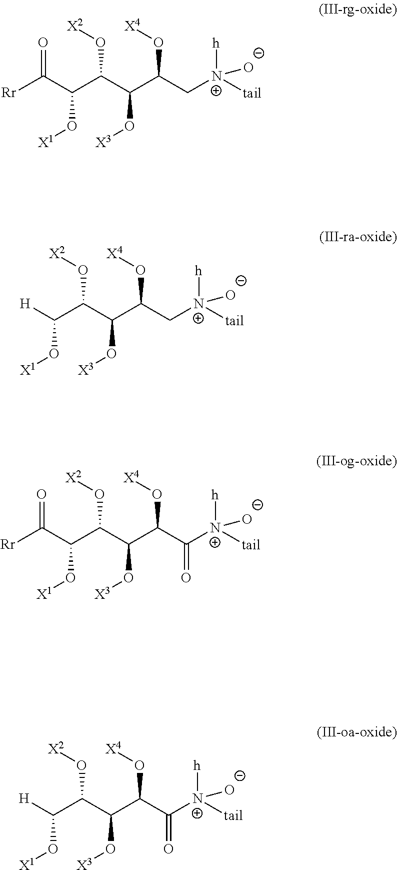

Compounds of general formulas as described herein that are not N-oxides can conveniently be used as precursors in the production of compounds of general formulas that are N-oxides.

### Compositions

Also provided is a composition comprising a compound as defined herein and a solvent or excipient. Such a composition may be called a composition according to the invention in the context of this application. In a preferred embodiment is provided a composition according to the invention, wherein said composition is a pharmaceutical composition. In a preferred embodiment is provided a composition according to the invention, wherein said composition is for use as a cleaning composition.

Compounds of the present invention confer desirable surfactant properties in a large and diverse range of products. For instance, the compounds were found to be surface active. The compounds are also biocompatible and biodegradable. The compounds of the present invention may confer beneficial properties already when used in low relative amounts. On the other hand, embodiments are envisaged, wherein the compound of the present invention constitutes a major ingredient. Hence, the relative amount of the compound in a composition according to the invention may vary over a wide range. In embodiments of the invention, a composition is provided comprising the compound according to the invention in an amount within the range of 0.05-99 wt. %, based on the total weight of the product, e.g. in an amount of at least 0.1 wt. %, at least 0.2 wt. %, at least 0.3 wt. %, at least 0.4 wt. %, at least 0.5 wt. %, at least 1 wt. %, at least 2 wt. %, at least 5 wt. % or at least 10 wt. % and/or in an amount of up to 50 wt. %, up to 25 wt. %, up to 20 wt. %, up to 15 wt. % or up to 10 wt. %. In embodiments of the invention, a composition is provided comprising the compound in an amount within the range of 0.1-50 wt. %, 0.2-25 wt. %, 0.3-20 wt. %, 0.4-15 wt. % or 0.5-10 wt. %.

In preferred embodiments, the composition or product is selected from the group consisting of pharmaceuticals, neutraceuticals, feed, food, cosmetics, detergents, fabric softeners, soaps, paints, adhesives, inks, anti-fogs, agrochemical products, herbicides, insecticides, biocides, cosmetics, shampoos, hair conditioners, toothpastes, coatings, drilling fluids, oilfield chemicals, emulsion polymerization systems and ferrofluids. More preferably, the product is selected from laundry detergent products, dishwashing products, personal care products, and hard surface cleaning products.

In embodiments of the invention, a composition as defined herein is provided, wherein the composition is a liquid, cream, gel, powder, spray, suspension, slurry, emulsion, lubricant or tablet.

In embodiments of the invention, a composition as defined herein is provided, wherein the composition comprises the compound according to the invention in addition to one, two, three, four or more component(s) selected from the group consisting of solvents, cosolvents, fragrance, softness extenders, other surfactants or emulsifiers, anti-redisposition agents, sequestrants, antiadherents, binders, coatings, colouring agents, disintegrants, flavors, glidants, lubricants, preservatives, sorbents, sweeteners, fillers, flow regulating agents, bulking agents, glycerides, glycols, monoesters of diols, wax, hydrophobic carriers, wetting agents, thickeners, chelating agents, abrasive agents, non-ionic surfactants, and ionic surfactants is provided.

In embodiments of the invention, a composition as defined herein is provided, wherein the composition is a laundry detergent product, comprising the compound of the present invention, in addition to one or more ingredients selected from the group consisting of phosphates, bleaching agents, optical brighteners, ionic surfactants, non-ionic surfactants, enzymes, alkaline salts, anti-foaming agents, complexing agents, perfumes, anti-caking agents, starches, gelling agents, emulsifiers, dispersing agents, dye transfer inhibitors, fabric softeners, colorants, etc. In preferred embodiments, a composition as defined herein is provided, wherein the composition is a laundry detergent product, comprising the compound of the present invention as a cosurfactant. In embodiments, this may be achieved by incorporating the compound of the present invention in an amount of less than 50 wt. % by weight of all surfactants in the composition, e.g. in an amount of less than 20 wt. %, less than 10 wt. %, less than 5 wt. %, less than 1 wt. %.

In embodiments of the invention, a composition as defined herein is provided, wherein the composition is a dishwashing product, comprising the compound of the present invention, in addition to one or more ingredients selected from the group consisting of phosphates, bleaching agents, ionic surfactants, non-ionic surfactants, enzymes, alkaline salts, anti-foaming agents, complexing agents, perfumes, anti-caking agents, starches, gelling agents, emulsifiers, dispersing agents, sand etc.

In embodiments of the invention, a composition as defined herein is provided, wherein the composition is a personal care product, comprising the compound of the present invention, in addition to one or more ingredients selected from the group consisting of hydrophobic carriers, cream bases, opacifiers, preservatives, chelating agents, emollients, emulsifiers, neutralizers, humectants, dyes, quenchers, proteins, thickeners, UV filters, vitamins, solubilizers, solvents, perfumes etc. By the term “personal care” is meant products such as skin cleansers, hair treatments (e.g. shampoos, mousses and conditioners), depilatories, skin lightening products, and leave-on skin lotions and creams. These products may be delivered from wipes (e.g. nonwoven substrates), liquids, gels, pumps or stick format.

In embodiments of the invention, a composition as defined herein is provided, wherein the composition is a hard surface cleaning product, comprising the compound of the present invention, in addition to one or more ingredients selected from the group consisting of chelating agents, other surfactants, wetting agents, solvents, diluents, acids, disinfectants, hydrophilic polymers, solvents, abrasives, inorganic absorbent materials, bleaching agents, perfume etc.

In embodiments of the invention, a composition as defined herein is provided, wherein the composition is an agrochemical product, comprising the compound of the present invention, in addition to one or more ingredients selected from the group consisting of pesticides, insecticides, fungicides, fertilizer compositions.

In embodiments of the invention, a composition as defined herein is provided, wherein the composition is a pharmaceutical product, comprising the compound of the present invention, in addition to one or more active pharmaceutical ingredients (‘APIs’), such as the compounds included in the Anatomical Therapeutic Chemical (ATC) classification system, maintained by the World Health Organization.

In embodiments of the invention, a composition as defined herein is provided, wherein the composition is a nutraceutical or food supplement product, comprising the polyhydroxy acid amide of the present invention, in addition to with one or more physiologically active ingredients, such as vitamins, minerals, trace elements, and/or one or more food-grade excipients, such as a compound which is recognized by the U.S. Food & Drug administration as GRAS (Generally Recognized as safe). In some embodiments of the invention said physiologically active ingredient is poorly water-soluble.

In embodiments of the invention, a composition as defined herein is provided, wherein the composition is a descaling product, comprising the compound of the present invention, in addition to one or more ingredients selected from the group consisting of descaling agents, complexing agents, chelating agents, binders, fillers, acidic compounds such as hydrochloric acid, acetic acid, citric acid, glycolic acid, formic acid, phosphoric acid and sulfamic acid.

In embodiments of the invention, a composition as defined herein is provided, wherein the composition is a chlorine product, comprising the compound of the present invention, in addition to one or more ingredients selected from the group of chlorine compounds, such as chlorinated isocyanurates, hypochlorite salts, chlorine dioxide, chloride of lime; etc.

In embodiments of the invention, a composition as defined herein is provided, wherein the composition is a mineral product, comprising the compound of the present invention, in addition to one or more ingredients selected from the group consisting of minerals of calcium, phosphorus, potassium, sodium, iron, cobalt, copper, zinc, manganese, molybdenum, iodine, and selenium, bromine, arsenic, nickel, fluorine, boron, lithium, strontium.

In embodiments of the invention, a composition as defined herein is provided, wherein the composition is a bleach product, comprising the compound of the present invention, in addition to one or more ingredients selected from the group consisting of chlorine-based bleaches, peroxide-based bleaches, reducing bleaches, peracetic acid, ozone and combinations thereof. Chlorine-based bleaches may comprise hypochlorite compounds such as calcium hypochlorite, chlorine dioxide, etc. Peroxide-based bleaches may comprise hydrogen peroxide, sodium percarbonate and sodium perborate. Reducing bleaches may comprise sodium dithionite and sodium oxymethylene sulfoxylate.

In embodiments of the invention, a composition comprising the compound according to the invention is provided, wherein the composition comprises an aqueous phase, typically liquid detergent formulations, shampoos, shower gels etc. In embodiments of the invention, a composition as defined herein is provided, having a water content within the range of 5-90 wt. %, based on the total weight of the composition, such as a water content of at least 5 wt. %, at least 10 wt. %, at least 20 wt. %, at least 30 wt. % or at least 40 wt. % and/or a water content of up to 90 wt. %, up to 80 wt. % or up to 70 wt. %.

In embodiments of the invention, a composition comprising the compound according to the invention is provided, wherein the composition further comprises at least one further component, which further component is poorly water-soluble. In some embodiments of the invention said at least one further component has a water solubility of less than 1 g/ml at a temperature of 20° C., e.g. a solubility of less than 0.1 g/ml, less than 0.01 g/ml or less than 0.001 g/ml. In embodiments of the invention, a composition as defined herein is provided, wherein the content of said further component is within the range of 0.0001-5 wt. %, based on the total weight of the composition, such as at least 0.0001 wt. %, at least 0.001 wt. %, at least 0.01 wt. %, at least 0.1 wt. % and/or up to 5 wt. %, up to 1 wt. %, up to 0.5 wt. %, up to 0.2 wt. %.

Reference is made to substances, components, or ingredients in existence at the time just before first contacted, formed in situ, blended, or mixed with one or more other substances, components, or ingredients in accordance with the present disclosure. A substance, component or ingredient identified as a reaction product, resulting mixture, or the like may gain an identity, property, or character through a chemical reaction or transformation during the course of contacting, in situ formation, blending, or mixing operation if conducted in accordance with this disclosure with the application of common sense and the ordinary skills of an average chemist. The transformation of chemical reactants or starting materials to chemical products or final materials is a continually evolving process, independent of the speed at which it occurs. Accordingly, as such a transformative process is in progress there may be a mix of starting and final materials, as well as intermediate species. Unless otherwise indicated herein, definitions of (relative) amounts of components concern the composition as is.

### METHODS AND USES

Compounds according to the invention van be produced using known synthetic methods, such as via reductive amination (see WO2019162469). Conveniently, compounds according to the invention can also be produced via oxidative amination. The inventors found that the use of a metal catalyst under mild conditions produces the compounds with a good yield, using eco-friendly reactants and reagents in an energy-efficient process. Accordingly, the invention provides a method for producing an amide-derivative of a saccharide, comprising the steps of:
i) providing a saccharide;
ii) providing a primary or secondary amine, preferably of general formula tail-NH-h wherein tail and h are as defined above;
iii) reacting the saccharide and the amine in the presence of a metal catalyst at a temperature of at most 60° C. to yield the amide-derivative;
iv) optionally isolating the amide-derivative.

Such a method is referred to herein as oxidative amination according to the invention. In preferred embodiments the steps are performed in numerical order.

### Step i) Provision of a Saccharide

The saccharide can be any suitable saccharide and the saccharide is preferably in pyranose or furanose form. Preferably the anomeric carbon is substituted with one hydrogen atom and one hydroxyl group. The saccharide can be a disaccharide or a monosaccharide or any other saccharide, although monosaccharides are most preferred for giving the highest yields or being more conveniently handled during work-up. In preferred embodiments the saccharide is derived from biomass, preferably from agrowaste, more preferably from vegetal pulp such as sugar beet pulp, and/or the saccharide is a monosaccharide, preferably arabinose or galacturonic acid, more preferably L-arabinose or D-galacturonic acid, most preferably D-galacturonic acid. Arabinose or galacturonic acid are particularly preferred saccharides, and galacturonic acid is most preferred.

Suitable biomass sources include those containing substantial quantities of arabinose and/or of galacturonic acid, such as hemicellulosic and pectin rich biomass. Materials may accordingly be utilized that, at present, are still mainly considered by-products in various industries. In preferred embodiments, the hemicellulose and pectin rich biomass is sugar beet pulp, which constitutes the production side stream from the sugar beet industry, for instance after other saccharides have been obtained from the beets. In alternative embodiments, the saccharide is obtained from a plant source such as from citrus fruits, tomatoes, chicory, potatoes, pineapple, apple, cranberries, grapes, carrots and the like. In some embodiments, a single saccharide is provided. In other embodiments, a mixture of more than one saccharides is provided. The saccharide can also be obtained from commercial suppliers, and can be part of a composition comprising further substances. The saccharide can be protected with protecting groups, although it can conveniently be used without protecting groups due to the mild reaction conditions. In preferred embodiments the saccharide is pure or substantially pure.

In some embodiments the provided saccharide has general formula (S):

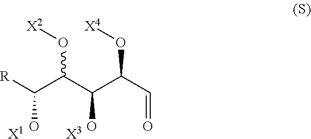

more preferably general formula (S2):

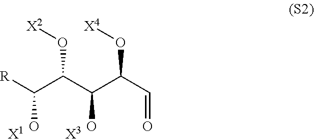

wherein within (S) or (S2), R is —H, —CH₂—Rr, or —C(═O)—Rr; Rr is —OH, —Oh¹, —NH₂, —NH(h¹), or —Nh¹h², wherein h¹ and h² are independently a linear, branched, or cyclic C₁₋₆ alkyl, alkenyl, or alkynyl moiety wherein each carbon atom is optionally substituted by halogen, alkoxy, or haloalkoxy; X¹, X², X³, and X⁴ are in each instance independently hydrogen or a linear, branched, or cyclic C₁₋₆ acyl moiety wherein each carbon atom is optionally substituted by halogen, alkoxy, or haloalkoxy, and wherein the acyl chain is optionally unsaturated, wherein two instances of X¹, X², X³, and X⁴ can together form a bridging moiety to form a five- or six-membered ring; or a salt thereof. A skilled person will understand that general formula (S) also encompasses corresponding furanoses and pyranoses, particularly pyranoses.

Within (S) or (S2), preferably R is —H, or —C(═O)—Rr, most preferably —C(═O)—Rr. Within (S) or (S2), preferably Rr is —OH or —Oh¹, wherein h¹ is a linear, branched, or cyclic C₁₋₆ alkyl, alkenyl, or alkynyl moiety wherein each carbon atom is optionally substituted by halogen, alkoxy, or haloalkoxy; most preferably it is —OH. Within (S) or (S2), preferably X¹, X², X³, and X⁴ are in each instance independently hydrogen or a linear, branched, or cyclic C₁₋₆ acyl moiety, more preferably hydrogen or acyl. In some embodiments within (S) or (S2) R is —H. In preferred embodiments within (S) or (S2) R is-C(═O)—OH.

### Step ii) Provision of an Amine

The amine provided in this step can be any suitable amine, keeping in mind that it should be primary or secondary. It is preferably primary. The tail of the resulting surfactant is a substituent on the amine as provided. Accordingly, the amine is preferably of general formula tail-NH-h wherein tail and h are as defined above. More preferably, the amine is a primary amine. In some embodiments the amine of step ii) is of general formula tail-NH₂, wherein tail is a linear C₃₋₂₀ alkyl.

Preferably, about two equivalents of the amine is provided as compared to the saccharide. Preferably, about 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.6, 2.7, 2.8, 2.9, 3, 3.5, 4 or more equivalents of amine are used relative to the saccharide. More preferably about 1.1-3 equivalents are used, still more preferably about 1.5-2.5, even more preferably about 1.8-2.2, such as about 2 equivalents are used.

Step iii) Reacting with a Metal Catalyst

The saccharide and the amine are then reacted in the presence of a metal catalyst such as a gold catalyst at a temperature of at most 60° C. to yield the amide-derivative. The metal catalyst can be any metal catalyst known to be suitable for the oxidation of carbohydrates, for instance as described by Eaqub Ali et al. (J. Nanomater., Vol. 2014, Article ID 192038). Metal catalysts are preferably heterogeneous catalysts. Examples of suitable catalysts are gold catalysts, platinum catalysts, palladium catalysts, iron catalysts, copper catalysts, cobalt catalysts, manganese catalysts, nickel catalysts, lead catalysts, and tellurium catalysts, preferably gold catalysts, platinum catalysts, palladium catalysts, iron catalysts, copper catalysts, cobalt catalysts, manganese catalysts, and nickel catalysts, more preferably gold catalysts, platinum catalysts, and palladium catalysts.

Metal catalysts can be bimetallic catalysts such as platinum bismuth catalysts, or gold platinum catalysts. Metal catalysts can be doped on supports, such as metal catalysts doped on bismuth or on carbon or on nanoparticles such as Al₂O₃ nanoparticles or ZrO₂ nanoparticles. Preferred metal catalysts are gold catalysts, platinum catalysts, palladium catalysts, more particularly gold catalysts doped on supports, platinum catalysts doped on supports, and palladium catalysts doped on supports.

In some embodiments the metal catalyst is a gold catalyst, platinum catalyst, more preferably a gold catalyst, a platinum catalyst, a platinum-bismuth catalyst, or a gold platinum catalyst.

Most preferably the metal catalyst is a gold catalyst. Examples of suitable gold catalysts are gold chlorides, gold oxides, and gold on a support. Examples of gold on a support are gold on titanium dioxide (AuTiO₂), gold nanoparticles on cellulose, gold on Al₂O₃, gold/palladium on Al₂O₃, gold on ZnO, gold on carbon, gold-palladium on carbon, gold on SiO₂ wherein the gold is preferably nanosized gold, and bimetallic gold/platinum nanoparticles. A gold catalyst is preferably a gold chloride or gold on a support, more preferably a gold on a metal oxide support such as AuTiO₂ or tetrachloroaurate such as KAuCl₄. Gold on a support is most preferred, particularly AuTiO₂. Preferably, about 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.6, 2.7, 2.8, 2.9, 3, 3.5, 4, 5, 6, 7, 8, 9, 10, 11, 12, 13, 14, 15 or more equivalents of catalyst are used relative to the saccharide. More preferably about 0.05-3 equivalents are used, still more preferably about 0.1-2, even more preferably about 0.5-1.5, such as about 1 equivalents are used. In preferred embodiments the gold catalyst is present at about 0.2-15 mol % such as at about 10 mol-%. For AuTiO₂ a preferred range is about 0.01% to about 0.05%.

The reacting is done in a suitable solvent, which can be protic or aprotic. Protic solvents are preferred for giving higher reaction yields. Examples of suitable protic solvents are lower alcohols, acetic acid, formic acid, and water, preferably a lower alcohol or water, most preferably a lower alcohol. Examples of suitable lower alcohols are ethanol, methanol, isopropanol, and butanol. Good results were obtained using methanol, ethanol, or mixtures of methanol and triethylamine in a ratio of 1:1 to 1:3 (either by volume or by equivalents), or of ethanol and triethylamine in those ratios.

An advantage of the method according to the invention is that it can be performed at relatively low temperatures, which reduces the energy requirements for performing the reaction, particularly at an industrially relevant scale. In fact, it was found that lower temperatures improved the yield of the reaction. Preferably the reacting of step iii) is performed at a temperature of about 20-60° C., preferably at about 20-40° C., more preferably at about 30-40° C. such as at about 35° C. The reacting is preferably performed at a temperature of at most 60° C., preferably at most 55° C., more preferably at most 50° C., even more preferably at most 45° C., still more preferably at most 40° C., optionally at most 35° C., and alternatively at most 30° C. A good balance of yield versus energy requirement was found with a reaction at about 30° C. or 40° C., such as at about 35° C.

In some embodiments the reacting of step iii) is performed in the presence of a base, preferably a non-nucleophilic base, more preferably an inorganic base such as a carbonate salt such as K₂CO₃ or Cs₂CO₃, and wherein the base is preferably present at about 1 to about 300 mol-%, such as at about 200 mol-%. A skilled person can select a suitable base, such as carbonate salts or bicarbonate salts. Preferably, about 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.6, 2.7, 2.8, 2.9, 3, 3.5, 4 or more equivalents of base are used relative to the saccharide. More preferably about 1-4 equivalents are used, still more preferably about 1.1-3, even more preferably about 1.5-2.5, such as about 2 equivalents are used. In other preferred embodiments 0.5-5 equivalents of base are used. In other preferred embodiments 0.5-5 equivalents of amine are used. In other preferred embodiments the non-nucleophilic base is an organic non-nucleophilic base such as a trialkylamine, for example diisopropylethylamine, particularly triethylamine, which is preferred when the gold catalyst is AuTiO₂.

An advantage of the method according to the invention is that the reacting can be performed in a single step. Accordingly in preferred embodiments the reacting of step iii) is performed in a single step. Another advantage of the method according to the invention is that no external stimulus such as irradiation is required. Accordingly in preferred embodiments the reacting of step iii) is performed under ambient background radiation. Another advantage of the method according to the invention is that no external oxidizing potential or oxidizing agent is requited. Accordingly in preferred embodiments the reacting of step iii) is performed without the addition of an oxidant. It should be understood that this does not imply that atmospheric oxygen should be excluded. In some embodiments, the reaction is performed under an 02 atmosphere, for instance using an 02 balloon.

Good results were obtained when the saccharide and the optional base (for instance 2 equivalents) were first dissolved in a lower alcohol such as methanol, after which the catalyst such as AuTiO₂ (for instance 1 equivalent) and the amine such as the tail-NH₂ (for instance 2 equivalents) were added. The mixture could then be stirred for about 48 hours at about 40° C.

After its formation in step iii), the compound according to the invention can be isolated using known techniques. It is not particularly important which technique is used. Examples of suitable techniques are counter-current solvent gradient purification or chromatography such as silica column chromatography or HPLC techniques. The compounds can optionally be purified using trituration or washing.

In preferred embodiments,
a) the reacting of step iii) is performed at a temperature of about 20-60° C., preferably at about 20-40° C., more preferably at about 30-40° C. such as at about 35° C.;
b) the metal catalyst is present at about 0.2-15 mol % such as at about 10 mol-%;
c) the reacting of step iii) is performed in a protic solvent;
d) the reacting of step iii) is performed in a single step;
e) the reacting of step iii) is performed without the addition of an oxidant; and/or
f) the reacting of step iii) is performed under ambient background radiation.

Also provided are compounds and composition as obtained and/or obtainable by the methods defined herein. Such compounds and/or compositions may be the same or may differ in some aspect(s) from compounds and/or compositions as described herein.

Also provided is the use of a compound and/or a compound or composition according to the invention as a surfactant or surface active component. In certain embodiments the use is provided of a compound and/or a composition according to the invention as a surfactant or surface active component in a product selected from the group consisting of laundry detergent products, dishwashing products, personal care products, hard surface cleaning products, and agricultural products.

Certain embodiments provide the use of a compound and/or a composition according to the invention for conferring and/or improving foaming properties, emulsifying properties and/or wetting properties in a product selected from the group consisting of laundry detergent products, dishwashing products, personal care products, hard surface cleaning products, food products, pharmaceutical products, and agricultural products.

### General Definitions

Herein, boldface can be used in variables to assist the reader; it does not imply further definition. In preferred embodiments, compounds and compositions according to the invention are for use in methods according to the invention, or are for use according to the invention. Each embodiment as identified herein may be combined together unless otherwise indicated.

When a structural formula or chemical name is understood by the skilled person to have chiral centers, yet no chirality is indicated, for each chiral center individual reference is made to all three of either the racemic mixture (having any enantiomeric excess), the pure R enantiomer, and the pure S enantiomer. Whenever a fragment of a molecule, often referred to as a moiety, is represented, a dotted or wavy line indicates which bond links it to the entirety of the molecule; alternately, an asterisk (*) indicates where the represented moiety is linked to the rest of the molecule. This asterisk does not imply an atom, and neither does a bond that is crossed by a dotted or wavy line convey information about which atom is at the non-moiety side of the bond. All this is known practice.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 10%, optionally 1% of the value.

All patent and literature references cited in the present specification are hereby incorporated by reference in their entirety.

### EXEMPLARY EMBODIMENTS THAT ARE PART OF THE DESCRIPTION

1. A compound of general formula (I):

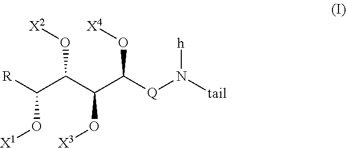

wherein
R is —H, —CH₂—Rr, or —C(═O)—Rr;
Rr is —OH, —Oh¹, —NH₂, —NH(h¹), or —Nh¹h², wherein h¹ and h² are independently a linear, branched, or cyclic C₁₋₆ alkyl, alkenyl, or alkynyl moiety wherein each carbon atom is optionally substituted by halogen, alkoxy, or haloalkoxy;
X¹, X², X³, and X⁴ are in each instance independently hydrogen or a linear, branched, or cyclic C₁₋₆ acyl moiety wherein each carbon atom is optionally substituted by halogen, alkoxy, or haloalkoxy, and wherein the acyl chain is optionally unsaturated, wherein two instances of X¹, X², X³, and X⁴ can together form a bridging moiety to form a five- or six-membered ring;
Q is —CH₂— or —C(═O)—;
h is a hydrogen or a linear, branched, or cyclic C₁₋₂₀ alkyl, alkenyl, or alkynyl moiety wherein each carbon atom is optionally substituted by halogen, alkoxy, or haloalkoxy;
or wherein h is an independently selected instance of tail;
tail is a linear, branched, or cyclic C₁₋₄₀ alkyl, alkenyl, or alkynyl moiety, wherein each carbon atom is optionally substituted by one or more halogen, -h^{t}, —O-h^{t}, —C(═O)Oh^{t}, —C(═O)N(h^{t})₂, —OC(═O)-h^{t}, —N(h^{t})C(═O)-h^{t}, ═O, or —N(h^{t})₂, wherein each instance of h^{t} is independently hydrogen or a linear, branched, or cyclic C₁₋₂₀ alkyl, alkenyl, or alkynyl moiety wherein each carbon atom of h^{t} is optionally substituted by halogen, alkoxy, or haloalkoxy;
or wherein tail is of general formula —(CH₂CH₂O)₁₄₅(CH₂)₀₋₂H;
or a salt or N-oxide thereof.
2. The compound according to embodiment 1, wherein it is of general formula (II):

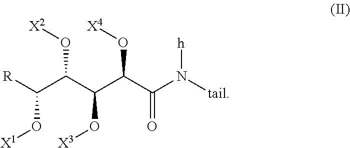

3. The compound according to embodiment 1 or 2, wherein it is of general formula (III-og), (III-oa), (III-rg), or (III-ra):

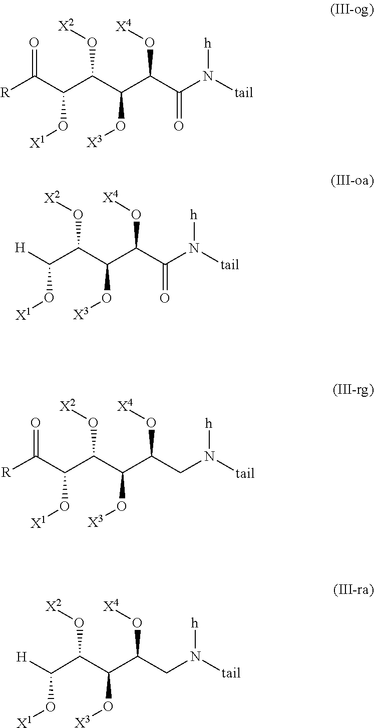

4. The compound according to any one of embodiments 1-3, wherein
Rr is —OH or —Oh¹, preferably —OH;
h¹ is —CH₃, —CH₂CH₃, —CH(CH₃)₂, or —C(CH₃)₃;
X¹, X², X³, and X⁴ are in each instance independently hydrogen or —C(═O)CH₃, and optionally each of X¹, X², X³, and X⁴ represent the same moiety, more preferably each of X¹, X², X³, and X⁴ represent hydrogen;
h is hydrogen or a linear, branched, or cyclic C₁₋₂₀ alkyl, alkenyl, or alkynyl moiety, preferably hydrogen, —CH₃, or —CH₂CH₃, more preferably hydrogen or —CH₃;
tail is a linear or branched C₁₋₂₂ alkyl, alkenyl, or alkynyl moiety, wherein up to two carbon atoms are optionally substituted by halogen, -h^{t}, —O-h^{t}, —C(═O)Oh^{t}, C(═O)N(h^{t})₂, —OC(═O)-h^{t}, —N(h^{t})C(═O)-h^{t}, ═O, or —N(h^{t})₂, wherein each instance of h^{t} is independently hydrogen or a linear or branched C₁₋₁₄ alkyl, alkenyl, or alkynyl moiety.
5. The compound according to any one of embodiments 1-4, wherein tail is a linear C₃₋₂₀ alkyl.
6. The compound according to any one of embodiments 1-5, wherein h is a linear, branched, or cyclic C₁₋₂₀ alkyl, alkenyl, or alkynyl moiety, preferably —CH₃, or —CH₂CH₃, more preferably —CH₃.
7. The compound according to any one of embodiments 1-6, wherein it is of general formula (IV) or (V):

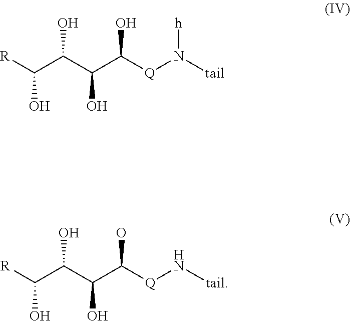

8. The compound according to embodiment 6, wherein tail is —(CH₂)₃—CH₃, —(CH₂)₅—CH₃, —(CH₂)₇—CH₃, —(CH₂)₉—CH₃, —(CH₂)₁₁—CH₃, —CH₂—CH(CH₃)—CH₃, —(CH₂)₁₀—COOH, —CH(CH₃)—(CH₂)₅—CH₃, —CH(CH₃)—(CH₂)₆—CH₃, —CH₂—CH(CH₂CH₃)—(CH₂)₃—CH₃, —(CH₂CH₂O)₃—CH₂CH₃, or —(CH₂CH₂O)₃—CH₃.
9. A composition comprising a compound as defined in any one of embodiments 1-8 and a solvent or excipient.
10. Method for producing an amide-derivative of a saccharide, comprising the steps of:
   i) providing a saccharide;
   ii) providing a primary or secondary amine, preferably of general formula tail-NH-h wherein tail and h are as defined in embodiment 1;
   iii) reacting the saccharide and the amine in the presence of a metal catalyst at a temperature of at most 60° C. to yield the amide-derivative;
   iv) optionally isolating the amide-derivative.
11. The method according to embodiment 10, wherein the saccharide is derived from biomass, preferably from agrowaste, more preferably from vegetal pulp such as sugar beet pulp,
and/or wherein the saccharide is a monosaccharide, preferably arabinose or galacturonic acid, more preferably L-arabinose or D-galacturonic acid, most preferably D-galacturonic acid.
12. The method according to embodiment 10 or 11, wherein the amine of step ii) is of general formula tail-NH₂, wherein tail is a linear C₃₋₂₀ alkyl.
13. The method according to any one of embodiments 10-12, wherein the metal catalyst is a gold catalyst, which is preferably a gold chloride, more preferably a gold oxide such as AuTiO₂ or tetrachloroaurate such as KAuCl₄.
14. The method according to any one of embodiments 10-13, wherein the reacting of step iii) is performed in the presence of a base, preferably a non-nucleophilic base, more preferably an inorganic base such as a carbonate salt such as K₂CO₃ or Cs₂CO₃, and wherein the base is preferably present at about 1 to about 300 mol-%, such as at about 200 mol-%.
15. The method according to any one of embodiments 10-14, wherein:
   a) the reacting of step iii) is performed at a temperature of about 20-60° C., preferably at about 20-40° C., more preferably at about 30-40° C. such as at about 35° C.;
   b) the metal catalyst is present at about 0.2-15 mol-% such as at about 10 mol-%;
   c) the reacting of step iii) is performed in a protic solvent;
   d) the reacting of step iii) is performed in a single step;
   e) the reacting of step iii) is performed without the addition of an oxidant; and/or
   f) the reacting of step iii) is performed under ambient background radiation.

### Examples

### Example 1—Synthetic Procedures

### General Procedures

NMR spectra were recorded on a Bruker Avance III 400 MHz or a Bruker 500 MHz spectrometer and the compounds were assigned using ¹H NMR, ¹³C NMR, ¹⁹F NMR, COSY, HSQCED and HMBC spectra. Chemical shifts were reported in parts per million (ppm.) relative to reference (CDCl₃: ¹H: 7.26 ppm. and ¹³C 77.16 ppm.; CD₃OD: ¹H: 3.31 ppm. and ¹³C 49.00 ppm.; D₂O: ¹H 4.79 ppm) NMR data are presented in the following way: chemical shift, multiplicity (s=singlet, bs=broad singlet, d=doublet, t=triplet, dd=doublet of doublets, ddd=doublet of doublet of doublets, dtd=doublet of triplet of doublets h=heptet, m=multiplet and/or multiple resonances) and coupling constants J in Hz. Reactions were monitored using TLC F254 (Merck KGaA) using UV absorption detection (254 nm) and by spraying them with 5% conc. H₂SO₄ in MeOH or cerium ammonium molybdate stain (Hannesian's stain) followed by charring at 300° C. Mass spectra were recorded on a JEOL AccuTOF CS JMS-T1000S (ESI) mass spectrometer. Purification by flash column chromatography was executed using silica gel 60 (Merck, 0.040-0.063 mm) or using automatic flash column chromatography on a Biotage Isolera Spektra One using SNAP or Silicycle cartridges (Biotage, 30-100 μm, 60 Å) 4-50 g. Reactions under protective atmosphere were performed under positive Ar·/N₂ flow in flame-dried flasks. Alternately, NMR spectra were recorded on a Bruker Avance III 400 MHz, Bruker 500 MHz, JEOL JNM-ECZ500R/S3 SuperCool or RoyaIHFX spectrometer and the compounds were assigned using 1H NMR, 13C NMR, 19F NMR, COSY, HSQCED and HMBC spectra. Chemical shifts were reported in parts per million (ppm.) relative to reference (CDCl₃: 1H: 7.26 ppm. and 13C 77.16 ppm.; CD3O_{D}: 1H: 3.31 ppm. and 13C 49.00 ppm.; D₂O: 1H 4.79 ppm) NMR data are presented in the following way: chemical shift, multiplicity (s=singlet, bs=broad singlet, d=doublet, t=triplet, dd=doublet of doublets, ddd=doublet of doublet of doublets, dtd=doublet of triplet of doublets h=heptet, m=multiplet and/or multiple resonances) and coupling constants J in Hz. Reactions were monitored using TLC F254 (Merck KGaA) using UV absorption detection (254 nm) and by spraying them with, 5% conc. H₂SO₄ in MeOH, ninhydrin or cerium ammonium molybdate stain (Hannesian's stain) followed by charring at 300° C. Mass spectra were recorded on a JEOL AccuTOF CS JMS-T1000S (ESI) mass spectrometer. Purification by flash column chromatography was executed using silica gel 60 (Merck, 0.040-0.063 mm) or using automatic flash column chromatography on a Biotage Isolera Spektra One using SNAP or Silicycle cartridges (Biotage, 30-100 μm, 60 Å) 4-50 g with 0-20% H₂O (1% acetic acid) in ACN as eluent.

General synthetic procedure 1: Synthesis of L-Ara derivatives via oxidative amination reaction (01-08) L-Ara (1 eq.) and K₂CO₃ (2 eq.) were dissolved in MeOH, subsequently N-alkylamine (2 eq.) and iodine (1.05 eq.) were added to the mixture. The mixture was stirred for 24/48/72/96 h at 40° C. Purification 01 and 02: Washed with EtOAc and recrystallized in H₂O/ACN. Purification 03 and 04: Washed with diethyl ether and recrystallized in H₂O and then in MeOH.

General synthetic procedure 2: Synthesis of D-GalA derivatives via oxidative amination reaction (09-16) D-GalA (1 eq.) and K₂CO₃ (2 eq.) were dissolved in dry MeOH, then N-alkylamine (2 eq.) and iodine (1.05 eq.) were added to the mixture. The mixture was stirred for 24/48/72/96 h at 40° C. The mixture was centrifuged and the pellet was washed several times with methanol until it turned white. Purification 09 and 10: The pellet was recrystallized in H₂O/ACN. Purification 11-13. The pellet was dissolved in H₂O and the pH was dropped until the precipitate was formed. After filtration, the residue was washed with MeOH.

General synthetic procedure 3: Gold catalysed reactions (01, 09, 11) The monosaccharide (1 eq.) and K₂CO₃ (2 eq.) were dissolved in MeOH, subsequently the gold particles 1% AuTiO₂ (1 eq) and N-alkylamine (2 eq.) were added to the mixture. The mixture was stirred for 48 h at 40° C. Resulting in starting material+product, purification until now only via column chromatography.

General synthetic procedure 4: Synthesis of L-Ara and D-GalA derivatives with secondary amines via oxidative amination reaction (17-22) L-Ara or D-GalA (1 eq.) and Cs₂CO₃ (2 eq.) were dissolved in MeOH, subsequently N-alkylamine (2 eq.) and slowly iodine (1.2 eq.) were added to the mixture. The mixture was stirred for 24/48/72/96 h at 40° C.

General synthetic procedure 5: Pd/c catalysed reactions of L-Ara and D-GalA derivatives via reductive amination (23-37) To a solution of L-Ara (1 eq.) or D-GalA (1.1 eq.) and N-alkylamine (1 eq.) in MeOH or EtOH, palladium on activated carbon (10 wt % loading) was added under an argon atmosphere. The reaction mixture was bubbled through with hydrogen for 15 min and was left stirring for 48/72/96 h at 20° C. The resulting mixture was purged of hydrogen by flushing with argon for 15 min.

General synthetic procedure 6: N-oxides (38-47) N-methyl-alkyl-D-galacturonicamine or N-methyl-alkyl-L-arabinamine (1 eq.), or N-methyl-alkyl-monosacharide-derivative-amine like N-methyloctyl-d-glucosamine (1 eq.), K₂CO₃ (2.1 eq.), or NaOH (3 eq), and H₂O₂ (35%) (3-12 eq.) were stirred at rt in H₂O for 8 h at rt or 50° C. The reaction mixture was quenched and concentrated in vacuo. The residue was dissolved in MeOH or EtOH and filtered. The Galacturonic acid derivatives could be precipitated with an acid and filtered of as purification step.

General synthetic procedure 7: To a solution of L-Ara (1 eq.) or D-GalA (1.1 eq.) and primary N-alkylamines (1 eq.) in MeOH or EtOH, palladium on activated carbon (10 wt % loading) was added under an argon atmosphere. The reaction mixture was bubbled through with hydrogen for 15 min and was left stirring for 48/72/96 h at 35° C. The resulting mixture was optionally purged of hydrogen by flushing with argon for 15 min. Subsequently, a formaldehyde 37% solution (1-3 eq.) was added.

The reaction mixture was optionally bubbled through with hydrogen for 15 min and then was left stirring for 48/72/96 h at 35° C. The resulting mixture was purged of hydrogen by flushing with argon for 15 min.

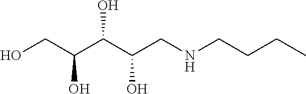

1-(N-butyl)-L-arabinonamide (also known as N-butyl-L-arabinonamide) (01): Via general synthetic procedure 1 starting from L-Ara (100 g, 666 mmol). Crystallization (ACN in H₂O) of the residue gave 1 (86.3 g, 60%) as an ivory solid. Via general synthetic procedure 3 starting from L-Ara (200 mg, 1.33 mmol). Column chromatography (20% H₂O in ACN) of the residue gave 1 (120 mg, 60%). ¹H NMR (500 MHz, D₂O) δ 4.47 (s, H-2 1H), 3.93 (m, H-3, H-5a, 2H), 3.83 (m, H-4, 1H), 3.74 (ddd, J=11.8, 6.3, 0.8 Hz, H-5b, 1H), 3.32 (hept, J=6.7 Hz, 2H), 1.64-1.54 (m, 2H), 1.46-1.34 (m, 2H), 0.96 (td, J=7.4, 0.8 Hz, 3H); ¹³C NMR (126 MHz, D₂O) δ 175.00 (C═O), 71.51 (C-3), 70.86 (C-2), 70.72 (C-4), 63.04 (C-5), 38.94, 30.65, 19.38, 13.09; HRMS (m/z): [M+Na]⁺ calcd for C₉H₁₉NO₅, 244.12; found, 244.11609.

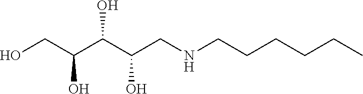 [SMILES: CCCCCCNC[C@H](O)[C@@H](O)[C@@H](O)CO]

1-(N-hexyl)-L-arabinonamide (also known as N-hexyl-L-arabinonamide) (02): Via general synthetic procedure 1 starting from L-Ara (100 g, 666 mmol). Crystallization (ACN in H₂O) of the residue gave 02 (74.4 g, 46%) as a white solid; ¹H NMR (500 MHz, D₂O) δ 4.40 (d, J=1.1 Hz, 1H H-2), 3.95 (m, 1H, H-3), 3.94 (m, 1H, H-5a), 3.84 (m, 1H, H-4), 3.76 (m, 1H, H-5b), 3.26 (t, 2H), 1.53 (m, 2H), 1.31 (m, 6H), 0.88 (t, 3H); ¹³C NMR (126 MHz, D₂O) δ 174.91 (C═O), 71.54 (C-3), 70.86 (C-2), 70.77 (C-4), 63.08 (C-5), 39.20, 30.81, 28.51, 25.76, 22.02, 13.42; HRMS (m/z): [M+Na]⁺ calcd for C₁₁H₂₃NO₅, 272.15; found, 272.14739.

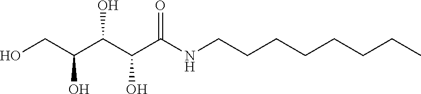 [SMILES: CCCCCCCCNC(=O)[C@H](O)[C@@H](O)[C@@H](O)CO]

1-(N-octyl)-L-arabinonamide (also known as N-octyl-L-arabinonamide) (03): Via general synthetic procedure 1 starting from L-Ara (100 g, 666 mmol). Crystallization (ACN in H₂O) of the residue gave 03 (58.7 g, 32%) as a white solid. ¹H NMR (500 MHz, D₂O) δ 4.37 (d, J=1.5 Hz, 1H, H-2), 3.93-3.79 (m, 2H, H-3, H-5a), 3.78-3.54 (m, 2H, H-4, H-5b), 3.33-3.17 (m, 2H), 1.56 (q, J=7.2 Hz, 2H), 1.36-1.25 (m, 12H), 1.01-0.78 (m, 2H); ¹³C NMR (126 MHz, MeOD) δ 174.50 (C═O), 72.03 (C-3), 71.30 (C-2), 70.97 (C-3), 63.54 (C-4), 38.77, 31.59, 29.26, 29.16, 29.04, 28.97, 26.57, 22.30, 13.01; HRMS (m/z): [M+Na]⁺ calcd for C₁₃H₂₇NO₅, 300.18; found, 300.17869.

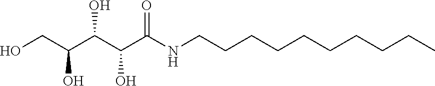 [SMILES: CCCCCCCCCCNC(=O)[C@H](O)[C@@H](O)[C@@H](O)CO]

1-(N-Decyl)-L-arabinonamide (also known as N-Decyl-L-arabinonamide) (04): Via general synthetic procedure 1 starting from L-Ara (1 g, 6.66 mmol). Crystallization (ACN in H₂O) of the residue gave 04 (1.36 g, 67%) as a white solid. ¹H NMR (500 MHz, D₂O) δ 4.28 (d, J=1.5 Hz, 1H, H-2), 3.82-3.67 (m, 2H, H-3, H-5a), 3.65-3.52 (m, 2H, H-4, H-5b), 3.25-3.12 (m, 2H), 1.51-1.43 (m, 2H), 1.25 (t, J=8.6 Hz, 16H), 0.88-0.77 (m, 3H); ¹³C NMR (126 MHz, D₂O) δ 175.00, 72.03, 71.31, 70.97, 63.55, 38.77, 31.69, 29.30 (2×), 29.16 (2×), 29.07, 29.04, 26.56, 13.00; HRMS (m/z): [M+Na]⁺ calcd for C₁₅H₃₁NO₅, 328.21; found, 328.20999.

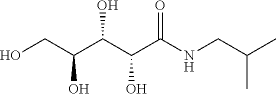

1-(N-isobutyl)-L-arabinonamide (also known as N-isobutyl-OH L-arabinonamid) (05): Via general synthetic procedure 1 starting from L-Ara (100 g, 666 mmol). Crystallization (ACN in H₂O) of the residue gave 05 (200 mg, 45%) as a white solid; ¹H NMR (500 MHz, D₂O) δ 4.36 (d, J=1.5 Hz, 1H, H-2), 3.85-3.76 (m, 2H, H-3, H-5a), 3.71 (dt, J=5.8, 2.8 Hz, 1H, H-4), 3.61 (dd, J=11.8, 6.3 Hz, 1H, H-5b), 3.07 (dd, J=13.2, 6.9 Hz, 1H), 2.99 (dd, J=13.2, 6.9 Hz, 1H), 1.76 (hept, J=6.7 Hz, 1H), 0.83 (d, J=6.7 Hz, 6H); ¹³C NMR (126 MHz, D₂O) δ 175.11 (C═O), 71.49 (C-3), 70.84 (C-2), 70.69 (C-4), 63.00 (C-5), 46.47, 27.92, 19.20, 19.19.

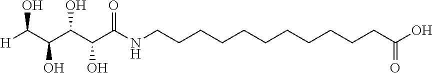

1-(N-(decanoic acid))-L-arabinonamide (also known as N-(decanoic acid)-L-arabinonamide) (06): Via general synthetic procedure 1 starting from L-Ara (1 g, 6.66 mmol). Crystallization (ACN in H₂O) of the residue gave 06 (604 mg, 26%) as a white solid. TLC (ACN/H₂O, 4/1, v/v): Rf=0.58; ¹H NMR (500 MHz, D₂O) δ 4.22 (d, J=1.7 Hz, 1H), 3.86-3.80 (m, 2H), 3.71 (ddd, J=8.7, 6.3, 2.9 Hz, 1H), 3.65 (dd, J=11.6, 6.3 Hz, 1H), 2.56 (t, J=7.1 Hz, 2H), 2.15 (t, J=7.5 Hz, 2H), 1.58-1.48 (m, 2H), 1.40 (t, J=7.1 Hz, 2H), 1.28 (s, 12H), ¹³C NMR (126 MHz, D₂O) δ 72.47, 71.66, 71.35, 63.21, 40.57, 37.66, 31.80, 28.72, 28.65, 28.58, 28.53, 28.50, 26.05, 25.89. HRMS (m/z): [M+Na]⁺ calcd for C₁₇H₃₃NO₇, 244.13; found, 372, 19955.

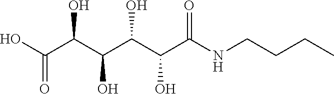 [SMILES: CCCCNC(=O)[C@H](O)[C@@H](O)[C@@H](O)[C@H](O)C(=O)O]

N-butyl-D-galactaric acid amide (09): Via general synthetic procedure 2 starting from D-GalA (100 g, 515 mmol). Crystallization (ACN in H₂O) of the residue gave 09 (15.4 g, 11%) as an ivory solid. Via general synthetic procedure 3 starting from D-GalA (200 mg, 1.03 mmol). Column chromatography (20% H₂O in ACN) of the residue gave 09 (29 mg, 11%). ¹H NMR (500 MHz, D₂O) δ 4.44 (d, J=1.2 Hz, 11H, H-2), 4.27 (d, J=1.2 Hz, 1H, H-5), 4.00 (d, J=3.1 Hz, 2H, H-3, H-4), 3.35-3.21 (m, 2H), 1.59-1.47 (m, 2H), 1.36 (h, J=7.4 Hz, 2H), 0.92 (td, J=7.4, 0.7 Hz, 3H); ¹³C NMR (126 MHz, D₂O) δ 179.34, 175.24, 71.29, 71.25, 71.09, 70.72, 38.84, 30.59, 19.29, 12.95; HRMS (m/z): [M+Na]⁺ calcd for C₁₀H₁₉NO₇, 288.11; found, 288.10432.

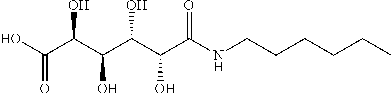 [SMILES: CCCCCCNC(=O)[C@H](O)[C@@H](O)[C@@H](O)[C@H](O)C(=O)O]

N-hexyl-D-galactaric acid amide (10): Via general synthetic procedure 2 starting from D-GalA (20 g, 103 mmol). Crystallization (ACN in H₂O) of the residue gave 10 (9.1 g, 30%) as a white solid. ¹H NMR (500 MHz, D₂O) δ 4.43 (s, 1H, H-2), 4.27 (s, 1H, H-5), 4.00 (d, J=3.0 Hz, 2H, H-3, H-4), 3.55-3.18 (m, 2H), 1.55 (q, J=7.0 Hz, 2H), 1.32 (d, J=5.5 Hz, 6H), 0.93-0.78 (m, 3H); ¹³C NMR (126 MHz, D₂O) δ 175.42 CONH, 179.41 COOH, 71.29, 71.25, 71.08, 70.71, 39.13, 30.68, 28.37, 25.61, 21.88, 13.28; HRMS (m/z): [M+Na]⁺ calcd for C₁₂H₂₃NO₇, 316.14; found 316.13833.

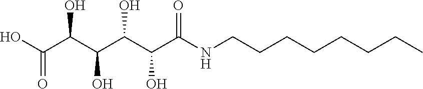 [SMILES: CCCCCCCCNC(=O)[C@H](O)[C@@H](O)[C@@H](O)[C@H](O)C(=O)O]

N-octyl-D-galactaric acid amide (11): Via general synthetic procedure 2 starting from D-GalA (10 g, 51.5 mmol). Crystallization (ACN in H₂O) of the residue gave 11 (6.8 g, 82%) as an ivory solid. Via general synthetic procedure 3 starting from D-GalA (200 mg, 1.03 mmol). Column chromatography (20% H₂O in ACN) of the residue gave 09 (42 mg, 15%). ¹H NMR (500 MHz, D₂O) δ 6 4.43 (s, 1H, H-2), 4.27 (d, J=1.3 Hz, 1H, H-5), 4.02-3.98 (m, 2H, H-3, H-4), 3.28 (td, J=6.9, 3.2 Hz, 2H), 1.56 (p, J=7.1 Hz, 2H), 1.41-1.21 (m, 1 OH), 0.90-0.85 (m, 3H); ¹¹C NMR (126 MHz, D₂O) δ 179.34, 175.20, 71.30, 71.26, 71.09, 70.71, 39.14, 31.07, 28.41, 28.33, 28.32, 25.94, 21.99, 13.38; HRMS (m/z): [M+Na]⁺ calcd for C₁₄H₂₇NO₇, 344.17; found, 344.16970.

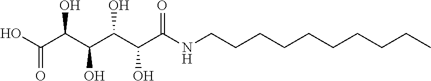 [SMILES: CCCCCCCCCCNC(=O)[C@H](O)[C@@H](O)[C@@H](O)[C@H](O)C(=O)O]

N-decyl-D-galactaric acid amide (12): Via general synthetic procedure 2 starting from D-GalA (10.0 g, 51.5 mmol). The reaction mixture was cooled down to room temperature and washed several times with MeOH using a centrifuge. Subsequently, the pellets were collected and dissolved in H₂O. The pH of the solution was dropped until a precipitate was formed. The resulting suspension was filtered, and the residue was washed with MeOH to yield 12 (3.9 g, 18%) as an ivory solid. HRMS (m/z): [M+Na]⁺ calcd for C₁₆H₃₁NO₇, 372.20; found, 372.20014; water solubility was relatively low for compound (12).

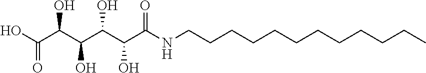 [SMILES: CCCCCCCCCCCCNC(=O)[C@H](O)[C@@H](O)[C@@H](O)[C@H](O)C(=O)O]

N-dodecyl-D-galactaric acid amide (13): Via general synthetic procedure 2 starting from D-GalA (10.0 g, 51.5 mmol). The reaction mixture was cooled down to room temperature and washed several times with MeOH and EtOAc using a centrifuge. Subsequently, the pellets were collected and dissolved in H₂O. The pH of the solution was dropped until a precipitate was formed. The resulting suspension was filtered, and the residue was washed with MeOH to yield 13 (4.6 g, 23.5%) as an ivory solid. HRMS (m/z): [M+Na]⁺ calcd for C₁₈H₃₅NO₇, 400.23; found, 400.23228; water solubility was relatively low for compound (13).

Compounds (09) through (13) can also be described as the corresponding 1-(N-alkyl)-D-galactaric acid monoamides.

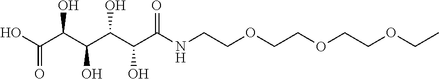 [SMILES: CCOCCOCCOCCNC(=O)[C@H](O)[C@@H](O)[C@@H](O)[C@H](O)C(=O)O]

N-2-(2-(2-ethoxyethoxy)ethoxy)ethyl-D-galactaric acid amide (15): Via general synthetic procedure 2 starting from D-GalA (1 g, 5 mmol). Column chromatography (20% H₂O in ACN) of the residue gave 15 (285 g, 15%) as a solid. TLC (ACN/H₂O (1% AA), 4/1, v/v): Rf=0.71; ¹H NMR (500 MHz, D₂O) δ 4.46 (s, 1H), 4.29 (s, 1H), 4.02 (d, J=1.9 Hz, 2H), 3.70 (m, 12H), 3.50 (tq, J=8.6, 5.4, 4.3 Hz, 2H), 1.20 (t, 3H). ¹³C NMR (126 MHz, D₂O) δ 177.43, 174.74, 71.22 (2×), 71.13, 70.80, 69.59 (2×), 69.47, 68.87, 66.43, 66.37, 39.11, 14.04. HRMS (m/z): [M+Na]⁺ calcd for C₁₄H₂₇NO₁₀, 392.15; found, 392.15292

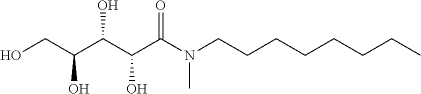 [SMILES: CCCCCCCCN(C)C(=O)[C@H](O)[C@@H](O)[C@@H](O)CO]

N-methyl-N-octyl-L-arabinonamide (17): Via general synthetic procedure 4 starting from L-Ara (1 g, 6.66 mmol). The mixture was concentrated under vacuo at 37 C. The residue was centrifuged with heptane (3 times), the pellet was dissolved in water (pH 14). EtOAc was added to the mixture and washed several times. The combined organic layers were concentrated under vacuo. The residue gave 17 (669 mg, 35%) as a white solid. TLC (ACN/H₂O, 4/1, v/v): Rf=0.7; ¹H NMR (500 MHz, CD₃OD) 4.81 (t, J=1.6 Hz, 1H, H-2), 3.88-3.62 (m, 4H, H-3, H-4, H-5a, H5-b), 3.55-3.25 (m, 2H, CH₂), 3.09 (s, 1.5H, Me), 2.97 (s, 1.5H, Me), 1.61 (m, 2H), 1.40-1.30 (m, 10H), 0.98-0.89 (m, 3H); 13C NMR (126 MHz, MeOD) δ 173.01, 71.09, 70.95, 67.43, 63.38, 48.66, 33.12, 31.59, 29.07, 28.96, 27.88, 26.50, 26.21, 22.30, 13.01. Compound (17) is well soluble in water and in methanol and has good foaming behaviour. HRMS (m/z): [M+Na]⁺ calcd for C₁₄H₂₉NO₅, 314.20; found, 314.19384.

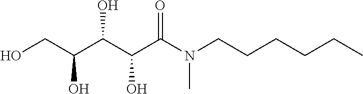 [SMILES: CCCCCCN(C)C(=O)[C@H](O)[C@@H](O)[C@@H](O)CO]

N-methyl-N-hexyl-L-arabinonamide (18): Via general synthetic procedure 4 starting from L-Ara (1 g, 6.66 mmol). The mixture was concentrated under vacuo at 37 C. The residue was centrifuged with heptane (3 times), the pellet was dissolved in water (pH 14). EtOAc was added to the mixture and washed several times. The combined organic layers were concentrated under vacuo and freeze dried in H₂O. The residue gave 18 (380 mg, 22%) as a white solid. TLC (ACN/H₂O, 4/1, v/v): Rf=0.7; ¹H NMR (500 MHz, CD₃OD) δ 4.80 (d, J=1.4 Hz, 1H), 3.86-3.77 (m, 1H), 3.75-3.61 (m, 3H), 3.41 (t, J=7.6 Hz, 2H), 3.02 (d, J=56.6 Hz, 3H), 1.69-1.54 (m, 2H), 1.35 (q, J=4.3, 3.9 Hz, 6H), 1.00-0.87 (m, 3H). ¹³C NMR (126 MHz, MeOD) δ 172.82, 71.85, 71.09 67.43, 63.34, 48.64, 33.75, 31.35, 27.82, 26.48, 22.22, 12.92. Compound (18) is well soluble in water and in methanol.

HRMS (m/z): [M+Na]⁺ calcd for C₁₂H₂₅NO₅, 286.17; found, 286.16095.

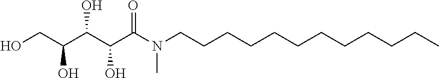 [SMILES: CCCCCCCCCCCCN(C)C(=O)[C@H](O)[C@@H](O)[C@@H](O)CO]

N-methyl-N-dodecyl-L-arabinonamide (19): Via general synthetic procedure 4 starting from L-Ara (1 g, 6.66 mmol). The mixture was concentrated under vacuo at 37 C. The residue was centrifuged with heptane (3 times), the filtrates were combined. In the filtrate a precipitated occurred, which was filtered. The residue was washed with heptane and freeze dried in H₂O. The residue gave 19 (1235 mg, 53%) as a white solid. TLC (ACN/H₂O, 4/1, v/v): Rf=0.7; ¹H NMR (500 MHz, CD₃OD) δ 4.88 (d, J=1.3 Hz, 1H), 3.80 (dd, J=11.1, 2.9 Hz, 1H), 3.76-3.62 (m, 3H), 3.41 (td, J=7.2, 3.0 Hz, 2H), 3.07 (s, 3H), 1.70-1.51 (m, 2H), 1.47-1.22 (m, 18H), 0.92 (t, J=6.9 Hz, 3H). ¹³C NMR (126 MHz, MeOD) δ 13C NMR (126 MHz, MeOD) δ 179.04, 71.71, 70.94, 67.14, 63.20, 48.70, 33.14, 31.66, 29.35, 29.13, 29.05 (2×), 27.95 (2×), 26.54, 26.23, 22.32, 13.02. Compound (19) is well soluble in water and in methanol and in ethyl acetate. =HRMS (m/z): [M+Na]⁺ calcd for C₁₈H₃₇NO₅, 370.27; found, 370.25860.

Compounds (17), (18), and (19) can also be described as the corresponding 1-(N-methyl-N-alkyl)-L-arabinonamides.

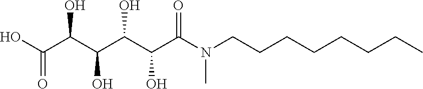 [SMILES: CCCCCCCCN(C)C(=O)[C@H](O)[C@@H](O)[C@@H](O)[C@H](O)C(=O)O]

N-methyl-N-octyl-D-galactaric acid amide (20): Via general synthetic procedure 4 starting from D-GalA (1 g, 5.15 mmol). The mixture was concentrated under vacuo at 37 C. The residue was centrifuged with heptane (3 times), the pellet was dissolved in water (pH 14). EtOAc was added to the mixture and washed several times. The combined water layers were concentrated under vacuo/or freeze dried and the residue was dissolved in EtOAc, to remove the salt. The residue gave 20 (511 mg, 30%) as a white solid. TLC (ACN/H₂O, 4/1, v/v): Rf=0.7; ¹H NMR (500 MHz, D₂O)¹H NMR (500 MHz, MeOD) δ 4.80 (d, 1H, H-2), 4.49 (dd, J=4.8, 1.5 Hz, 1H, H-5), 4.08 (ddd, J=9.6, 4.4, 1.6 Hz, 1H, H-4), 3.83 (m, 1H, H-3), 3.42 (dd, J=8.4, 6.7 Hz, 2H, CH₂), 3.08 (s, 1.5H, Me), 2.98 (s, 1.5H, Me), 1.75-1.55 (m, 2H), 1.41-1.24 (m, 10H), 0.98-0.88 (m, 3H). ¹¹C NMR (126 MHz, MeOD) δ 174.43, 71.47, 70.40, 69.72, 67.06, 48.34, 33.29, 31.59, 29.09, 27.90, 26.50, 26.21, 22.30, 13.02. Compound (20) is well soluble in water and in methanol and has good foaming behaviour. HRMS (m/z): [M+Na]⁺ calcd for C₁₅H₂₉NO₇, 358.19; found, 358.18453

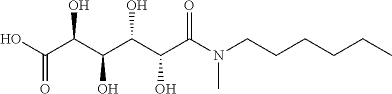 [SMILES: CCCCCCN(C)C(=O)[C@H](O)[C@@H](O)[C@@H](O)[C@H](O)C(=O)O]

1-(N-methyl-N-hexyl)-D-galactaric acid monoamide (21): Via general synthetic procedure 4 starting from D-GalA g, 5.15 mmol). The mixture was concentrated under vacuo at 37 C. The residue gives 21 as a white solid. TLC (ACN/H₂O (Acid), 4/1, v/v): Rf=0.7.

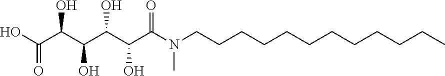 [SMILES: CCCCCCCCCCCCN(C)C(=O)[C@H](O)[C@@H](O)[C@@H](O)[C@H](O)C(=O)O]

N-methyl-N-dodecyl-D-galactaric acid amide (22): Via general synthetic procedure 4 starting from D-GalA (1 g, 5.15 mmol). The mixture was concentrated under vacuo at 37 C. The residue was dissolved in water (pH 14) and EtOAc was added. Precipitate was formed and filtered. The residue was again dissolved in water (pH 14), EtOAc was added to the mixture and washed several times. The combined water layers were washed with amberlite H+ and freeze dried. The residue gave 22 (942 mg, 47%) as a white solid. TLC (ACN/H₂O (Acid), 4/1, v/v): Rf=0.6; ¹H NMR (500 MHz, MeOD) δ 4.82 (d, 1H, H-2), 4.51 (dd, J=4.6, 1.5 Hz, 1H, H-5), 4.13-4.06 (m, 1H, H-4), 3.84 (ddd, J=16.4, 9.5, 1.2 Hz, 1H, H-3), 3.45 (m, 2H), 3.00 (d, 3H), 1.68-1.57 (m, 2H), 1.31 (m, 18H), 0.92 (t, J=6.9 Hz, 3H); ¹¹C NMR (126 MHz, MeOD) δ 175.70 (COOH), 172.80 (CONH₂), 71.42 (C-4), 70.51 (C-3), 69.87 (C-5), 66.78 (C-2), 47.88, 33.60, 32.25, 31.69, 29.23 (6×), 26.50, 26.36, 22.13, 12.98. HRMS (m/z): [M+Na]⁺ calcd for C₁₉H₃₇NO₇, 414.26; found, 414.24812. Compound (22) is well soluble in water and in methanol and has good foaming behaviour.

Compounds (20) and (22) can also be described as the corresponding 1-(N-methyl-N-alkyl)-D-galactaric acid monoamides.

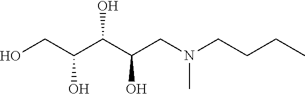

N-methyl-N-butyl-L-arabinamine (23b) (rAra1.4): Via general synthetic procedure 7 starting from L-Ara (1 g). The Pd/C catalyst was filtered off over Celite and washed with MeOH. The filtrate was concentrated in vacuo to yield compound 23b (1627 mg, 80%) as an off-white solid. ¹H NMR (500 MHz, MeOD) δ 3.96 (ddd, J=7.3, 4.9, 2.2 Hz, 1H, H-2), 3.77 (dd, J=11.0, 3.4 Hz, 1H, H-5a), 3.64 (ddd, J=8.1, 5.9, 3.4 Hz, 1H, H-4), 3.59 (dd, J=11.1, 5.9 Hz, 1H, H-5b), 3.38 (dd, J=8.2, 2.3 Hz, 1H, H-3), 2.63 (dd, J=12.9, 7.5 Hz, 1H, H-1a), 2.54-2.39 (m, 3H, H-1b CH₂), 2.30 (s, 3H), 1.52-1.43 (m, 2H), 1.36-1.27 (m, 2H), 0.92 (t, J=7.4 Hz, 3H). ¹³C NMR (126 MHz, METHANOL-D₄) ¹³C NMR (126 MHz, METHANOL-D₄) δ. 72.80, 71.83, 67.24, 63.74, 60.74, 57.88, 41.68, 28.71, 20.32, 13.04. HRMS (m/z): [M+H]⁺ calculated for C₁₀H₂₃NO₄, 222.16; found, 222.27

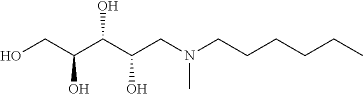 [SMILES: CCCCCCN(C)C[C@H](O)[C@@H](O)[C@@H](O)CO]

N-methyl-N-hexyl-L-arabinamine (23) (rAra1.6): Via general synthetic procedure 5 starting from L-Ara (2.0 g, 13.3 mmol). The Pd/C catalyst was filtered off on Celite and washed with MeOH. The filtrate was concentrated in vacuo to yield 23 (3.1 g, 94%) as a white solid. TLC (ACN/H₂O, 4/1, v/v): Rf=0.11; ¹H NMR (500 MHz, MeOD) δ 4.00 (ddd, J=7.4, 4.9, 2.2 Hz, 1H, H-2), 3.81 (dd, J=11.0, 3.4 Hz, 1H, H-5), 3.71-3.66 (m, 1H, H-4), 3.66 (m, 1H, H-5), 3.42 (dd, J=8.2, 2.3 Hz, 1H, H-3), 2.66 (dd, J=12.9, 7.5 Hz, 1H, H-1), 2.57-2.39 (m, 3H, H-1), 2.33 (s, 3H), 1.58-1.48 (m, 2H), 1.41-1.30 (m, 6H), 0.97-0.89 (m, 3H); ¹³C NMR ¹³C NMR (126 MHz, MeOD) δ 72.77 (C-3), 71.77 (C-4), 67.23 (C-2), 63.68 (C-5), 60.71 (C-1), 58.13, 41.63, 31.54, 26.84, 26.49, 22.28, 12.98; HRMS (m/z): [M+Na]⁺ calcd for C₁₂H₂₇NO₄, 272.18; found, 272.18291.

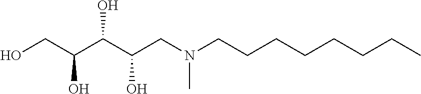 [SMILES: CCCCCCCCN(C)C[C@H](O)[C@@H](O)[C@@H](O)CO]

N-methyl-N-octyl-L-arabinamine (24) (rAra1.8): Via general synthetic procedure 5 starting from L-Ara (2.0 g, 13.3 mmol). The Pd/C catalyst was filtered off on Celite and washed with MeOH. The filtrate was concentrated in vacuo to yield 24 (3.3 g, 99%) as a yellow solid. TLC (ACN/H₂O, 4/1, v/v): Rf=0.39; ¹H NMR (500 MHz, MeOD) δ 4.01 (ddd, J=7.3, 4.9, 2.3 Hz, 1H, H-2), 3.81 (dd, J=11.0, 3.4 Hz, 1H, H-5), 3.69 (ddd, J=8.2, 5.9, 3.4 Hz, 1H, H-4), 3.64 (dd, J=11.0, 5.8 Hz, 1H, H-5), 3.42 (dd, J=8.2, 2.2 Hz, 1H, H-3), 2.67 (dd, J=12.9, 7.5 Hz, 1H, H-1), 2.58-2.40 (m, 3H, H-1), 2.34 (s, 3H), 1.58-1.49 (m, 2H), 1.40-1.28 (m, 10H), 0.95-0.88 (m, 3H); ¹³C NMR (126 MHz, MeOD) δ 72.75 (C-3), 71.76 (C-4), 67.19 (C-2), 63.68 (C-5), 60.67 (C-1), 58.11, 41.61, 31.61, 29.25, 29.01, 27.16, 26.47, 22.31, 13.02; HRMS (m/z): [M+H]⁺ calcd for C₁₄H₃₁NO₄, 278.23; found, 278.23295.

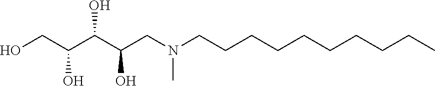 [SMILES: CCCCCCCCCCN(C)C[C@@H](O)[C@@H](O)[C@H](O)CO]

N-methyl-N-decyl-L-arabinamine (24b) (rAra1.10): Via general synthetic procedure 7 starting from L-Ara (1 g, 7.6 mmol). The Pd/C catalyst was filtered off over Celite and washed with EtOH. The filtrate was concentrated in vacuo to yield compound 24b (3.88 gr, 98%) as an off-white solid. TLC (ACN/H₂O (1% AA), 4/1, v/v): R_{f}=0.15 ¹H NMR (500 MHz, MeOD) δ 3.95 (dq, J=8.3, 3.0 Hz, 1H, H-2), 3.77 (dd, J=11.0, 3.4 Hz, 1H, H-5a), 3.68-3.62 (m, 1H, H-4), 3.56 (s, 1 Hz, H-5b), 3.37 (ddd, J=11.1, 8.3, 2.5 Hz, 1H, H-3), 2.61 (td, J=12.9, 8.3 Hz, 1H, H-1b), 2.54-2.35 (m, 3H, H-1b, CH₂), 2.29 (s, 3H), 1.48 (q, J=9.4 Hz, 2H), 1.28 (q, J=8.4 Hz, 14H), 0.92-0.81 (m, 3H). ¹³C NMR (126 MHz, METHANOL-D₄) ¹³C NMR (126 MHz, METHANOL-D₄) δ 72.82 (C-3), 71.83 (C-4), 67.26 (C-2), 63.74 (C-5), 60.74 (C-1), 58.17, 41.67, 31.72, 29.37 (3×), 29.11, 27.22, 26.55, 22.38, 13.09. HRMS (m/z): [M+H]⁺ calculated for C₁₆H₃₅NO₀, 306.25661; found, 306.26254

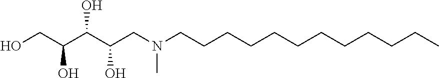 [SMILES: CCCCCCCCCCCCN(C)C[C@H](O)[C@@H](O)[C@@H](O)CO]

N-methyl-N-dodecyl-L-arabinamine (25) (rAra1.12): Via general synthetic procedure 5 starting from L-Ara (2.0 g, 13.3 mmol). The Pd/C catalyst was filtered off on Celite and washed with 3:1 EtOH/H₂O. The filtrate was concentrated in vacuo to yield 25 (3.8 g, 85%) as a white solid. TLC (ACN/H₂O, 4/1, v/v): Rf=0.22; ¹H NMR (500 MHz, MeOD) δ 4.00 (ddd, J=7.3, 4.9, 2.3 Hz, 1H, H-2), 3.81 (dd, J=11.0, 3.4 Hz, 1H, H-5), 3.72-3.66 (m, 1H, H-4), 3.63 (dd, J=11.0, 5.8 Hz, 1H, H-5), 3.42 (dd, J=8.2, 2.3 Hz, 1H, H-3), 2.65 (d, J=7.5 Hz, 1H, H-1), 2.56-2.41 (m, 3H, H-1), 2.33 (s, 3H), 1.53 (p, J=7.1, 6.6 Hz, 2H), 1.32 (d, J=10.9 Hz, 20H), 0.95-0.89 (m, 3H); ¹³C NMR (126 MHz, MeOD) δ 72.77 (C-3), 71.77 (C-4), 67.22 (C-2), 63.68 (C-5), 60.70 (C-1), 58.13, 41.63, 31.67, 29.38, 29.35, 29.34, 29.29, 29.07, 29.06, 27.17, 26.51, 22.33, 13.03; HRMS (m/z): [M+Na]⁺ calcd for C₁₈H₃₉NO₄, 356.28; found, 356.27714.

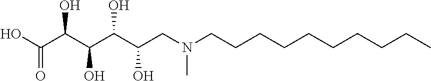 [SMILES: CCCCCCCCCCN(C)C[C@H](O)[C@@H](O)[C@@H](O)[C@H](O)C(=O)O]

N-methyldecyl-D-galactaric acid amine (25b) (rGala1.10): Via general synthetic procedure 7 starting from D-GalA (2 g, 10.30) mmol). The Pd/C catalyst was filtered off over Celite and washed with EtOH. The filtrate was concentrated in vacuo and freeze-dried to yield compound 25b (2.39 gr, 73%) as a white powder. TLC (ACN/H₂O (1% AA), 4/1, v/v): Rf=0.22. 1 H NMR (500 MHz, MeOD) δ 4.35-4.29 (m, 1H, H₅), 4.20 (d, J=1.9 Hz, 1H, H₃), 3.94 (dd, J=9.4, 1.9 Hz, 1H, H₄), 3.51-3.46 (m, 1H, H₂), 3.40 (dd, J=13.0, 10.3 Hz, 1H), 3.21 (d, J=11.9 Hz, 2H, H₁), 3.17 (d, J=5.2 Hz, 2H), 2.93 (s, 3H), 1.89-1.65 (m, 2H), 1.47-1.16 (m, 14H), 1.03-0.78 (m, 3H). 13C NMR (126 MHz, MeOD) δ 178.19 (COOH), 71.62 (C4), 71.42 (C2), 71.36 (C3), 64.60 (C5), 56.73 (C1), 59.17, 40.06, 31.70, 29.22, 29.17, 29.02, 28.87, 26.33, 23.66, 22.41, 13.02 HRMS (m/z): [M+H]⁺ calculated for C₁₇H₃₅NO₆, 350.24644; found, 350.25369. Alternately Via general synthetic procedure 7 starting from D-GalA (2 g, 5.72 mmol). The Pd/C catalyst was filtered off on Celite and washed with MeOH and H₂O. The filtrate was concentrated in vacuo to yield 25b (x g, x %) as an ivory solid. TLC (ACN/H₂O, 4/1, v/v): Rf=0.7. MS (m/z): [M+H]⁺ calcd for C₁₇H₃₅NO₆, 350.25; found, 350.19.

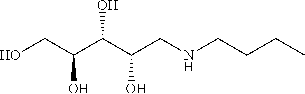

N-butyl-L-arabinamine (26): Via general synthetic procedure 5 starting from L-Ara (2.0 g, 13.3 mmol). The Pd/C catalyst was filtered off on Celite and washed with MeOH. The filtrate was concentrated in vacuo to yield 26 (2.7 g, 96%) as a white solid. TLC (ACN/H₂O, 4/1, v/v): Rf=0.06; ¹H NMR (500 MHz, D₂O) δ 3.91 (ddd, J=8.8, 4.2, 2.3 Hz, 1H, H-2), 3.75 (dd, J=11.8, 3.0 Hz, 1H, H-5), 3.68-3.62 (m, 1H, H-4), 3.60-3.55 (m, 1H, H-5), 3.44-3.38 (m, 1H, H-3), 2.80-2.61 (m, 2H, H-1), 2.56 (dt, J=9.8, 7.3 Hz, 2H), 1.45-1.37 (m, 2H), 1.30-1.21 (m, 2H), 0.83 (t, J=7.4 Hz, 3H); ¹³C NMR (126 MHz, D₂O) δ 72.02 (C-3), 70.98 (C-4), 68.40 (C-2), 62.95 (C-5), 51.19 (C-1), 48.16, 30.50, 19.75, 13.17; HRMS (m/z): [M+H]⁺ calcd for C₉H₂₁NO₄, 208.15; found, 208.15366. (This is a reference compound).

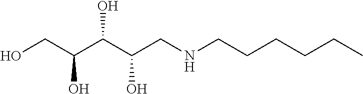 [SMILES: CCCCCCNC[C@H](O)[C@@H](O)[C@@H](O)CO]

N-hexyl-L-arabinamine (27) (rAra6): Via general synthetic procedure 5 starting from L-Ara (2.0 g, 13.3 mmol). The Pd/C catalyst was filtered off on Celite and washed with H₂O and MeOH. The filtrate was concentrated in vacuo to yield 27 (2.5 g, 81%) as a yellow solid. TLC (ACN/H₂O, 4/1, v/v): Rf=0.11; ¹H NMR (500 MHz, MeOD) δ 3.99 (ddd, J=8.1, 4.1, 2.2 Hz, 1H, H-2), 3.79 (ddd, J=10.7, 8.9, 3.2 Hz, 1H, H-5), 3.70-3.66 (m, 1H, H-4), 3.64 (ddd, J=11.0, 5.9, 3.2 Hz, 1H, H-5), 3.43 (dd, J=8.0, 2.2 Hz, 1H, H-3), 2.93-2.71 (m, 2H, H-1), 2.71-2.57 (m, 2H), 1.60-1.49 (m, 2H), 1.41-1.30 (m, 6H), 0.96-0.90 (m, 3H); ¹³C NMR (126 MHz, MeOD) δ 72.81 (C-3), 71.68 (C-4), 68.54 (C-2), 63.59 (C-5), 52.24 (C-1), 49.19, 31.50, 29.07, 26.67, 22.25, 12.96; HRMS (m/z): [M+H]⁺ calcd for C₁₁H₂₅NO₄, 236.18; found, 236.18584. (This is a reference compound).

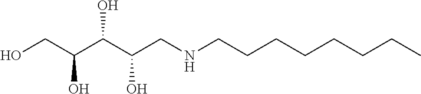 [SMILES: CCCCCCCCNC[C@H](O)[C@@H](O)[C@@H](O)CO]

N-octyl-L-arabinamine (28) (rAra8): N-octylamine (2 eq.) was added dropwise to a solution of L-Ara (2.0 g, 13.3 mmol) in MeOH. The reaction mixture was stirred for 24 h at 20° C. The resulting mixture was cooled to 0° C. and NaBH4 (1.5 eq.) was added stepwise. After stirring for 2.5 h, the pH of the reaction mixture was lowered to 1 by dropwise addition of 6 M HCl. The resulting suspension was filtered to yield 28 (1.8 g, 52%) as a white solid. TLC (ACN/H₂O, 4/1, v/v): Rf=0.12; ¹H NMR (500 MHz, EtOD) δ 4.25 (ddd, J=9.6, 3.3, 1.8 Hz, 1H, H-2), 3.83 (dd, J=11.4, 2.9 Hz, 1H, H-5), 3.73 (ddd, J=8.5, 5.6, 2.9 Hz, 1H, H-4), 3.69 (dd, J=11.4, 5.6 Hz, 1H, H-5), 3.51 (dd, J=8.7, 1.8 Hz, 1H, H-3), 3.30-3.15 (m, 2H, H-1), 3.12-3.05 (m, 2H), 1.81-1.71 (m, 2H), 1.35-1.24 (m, 10H), 0.92-0.85 (m, 3H); ¹³C NMR (126 MHz, EtOD) δ 71.29 (C-3), 70.80 (C-4), 65.78 (C-2), 63.05 (C-5), 50.49 (C-1), 47.89, 31.53, 28.85, 26.30, 26.20, 25.72, 22.31, 13.42; HRMS (m/z): [M+Na]⁺ calcd for C₁₃H₂₉NO₄, 286.20; found, 286.19846. (This is a reference compound).

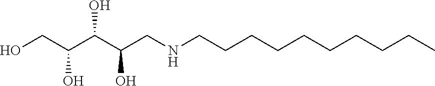 [SMILES: CCCCCCCCCCNC[C@@H](O)[C@@H](O)[C@H](O)CO]

N-decyl-L-arabinamine (28b) (rAra10): Via general synthetic procedure 5 starting from L-Ara (2.0 g, 13.3 mmol). The Pd/C catalyst was filtered off over Celite and washed with MeOH. The filtrate was concentrated in vacuo and subsequently washed with ethyl acetate. Acetone was added and the product precipitated as a white powder. Filtration was used to yield 28b (283 mg, 7%) as an off-white powder. TLC (ACN/H₂O, 4/1, v/v): Rf=0.67; ¹H NMR (500 MHz, MeOD) δ 3.96 (ddd, J=8.2, 4.1, 2.2 Hz, 1H, H-2), 3.80-3.57 (m, 3H, H-4, H-5), 3.39 (dd, J=8.0, 2.2 Hz, 1H, H₃), 2.83-2.69 (m, 1H, H-1), 2.68-2.54 (m 3H, H-1, CH₂), 1.51 (p, J=7.1 Hz, 2), 1.35-1.20 (m, 14H), 0.87 (t, J=6.8 Hz, 2H). ¹³C NMR (126 MHz, MeOD) δ 72.74 (C-3), 71.69 (C-4), 68.45 (C-2), 63.61 (C-5), 52.16 (C-1), 29.33, 29.27, 28.95, 26.99, 22.38, 13.11. HRMS (m/z): [M+H]⁺ calcd for C₁₅H₃₃NO₄, 292.24; found, 292.24982

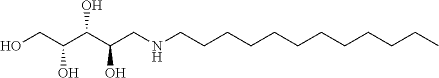 [SMILES: CCCCCCCCCCCCNC[C@@H](O)[C@@H](O)[C@H](O)CO]

N-dodecyl-L-arabinamine (28c) (rAra12): Via general synthetic procedure 5 starting OH from L-Ara (2.01 g, 13.4 mmol). The Pd/C catalyst was filtered off on Celite and washed with MeOH. The filtrate was concentrated in vacuo to yield 28c (1.155 g, 27%) as a yellow solid. TLC (ACN/H₂O, 4/1, v/v): Rf=0.56; ¹H NMR (500 MHz, MeOD) δ 3.84 (ddd, J=7.1, 5.5, 4.1 Hz, 1H, H2), 3.80-3.71 (m, 1H, H5a), 3.67-3.61 (m, 1H, H4), 3.61-3.55 (m, 1H, H5bz), 3.55-3.48 (m, 1H, H3), 2.87 (dd, J=12.2, 4.2 Hz, 1H, H1), 2.75-2.66 (m, 1H, H1), 2.66-2.53 (m, 2H), 1.50 (s, 2H), 1.33-1.25 (m, 18H), 0.87 (t, J=6.9 Hz, 3H). ¹³C NMR (126 MHz, MeOD) δ 74.40 (C3), 72.76 (C4), 70.28 (C2), 63.40 (C5), 51.17 (C1), 49.21, 31.73, 29.44, 29.41, 29.39, 29.35, 29.26, 29.11, 29.04, 27.02, 22.39, 13.15. HRMS (m/z): [M+H]⁺ calcd for C₁₇H₃₇NO₄, 320.27; found, 320.27941

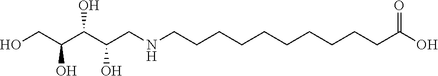 [SMILES: O=C(O)CCCCCCCCCCNC[C@H](O)[C@@H](O)[C@@H](O)CO]

N-(decanoic acid)-L-arabinamine (29): Via general synthetic procedure 5 starting from L-Ara (2.0 g, 13.3 mmol). The Pd/C catalyst was filtered off on Celite and washed with MeOH. The filtrate was concentrated in vacuo and dissolved in H₂O. The pH was lowered to 1 and a precipitate was formed. The resulting suspension was filtered to yield 29 (3.6 g, 90%) as a white solid. TLC (ACN/H₂O, 4/1, v/v): Rf=0.27; ¹H NMR (500 MHz, D₂O) δ 3.89-3.85 (m, 1H, H-2), 3.73 (ddd, J=11.6, 3.5, 0.8 Hz, 1H, H-5), 3.67-3.61 (m, 1H, H-4), 3.56 (ddd, J=11.5, 6.2, 0.9 Hz, 1H, H-5), 3.41 (ddd, J=7.8, 2.3, 0.9 Hz, 1H, H-3), 2.67 (ddd, J=12.7, 8.5, 0.8 Hz, 1H, H-1), 2.59 (ddd, J=12.7, 4.4, 0.9 Hz, 1H, H-1), 2.50 (dd, J=10.2, 7.3 Hz, 2H), 2.09 (td, J=7.4, 0.9 Hz, 2H), 1.43 (dt, J=28.1, 7.1 Hz, 4H), 1.27-1.19 (m, 14H); ¹³C NMR (126 MHz, D₂O) δ 184.32, 72.54 (C-3), 71.48 (C-4), 68.86 (C-2), 63.08 (C-5), 51.41 (C-1), 48.59, 37.69, 35.56, 28.77, 28.66, 28.63, 28.61, 28.55, 26.49, 25.93; HRMS (m/z): [M+H]⁺ calcd for C₁₆H₃₃NO₆, 336.23; found, 336.23814.

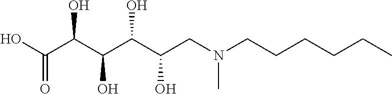 [SMILES: CCCCCCN(C)C[C@H](O)[C@@H](O)[C@@H](O)[C@H](O)C(=O)O]

N-methyl-N-hexyl-D-galacturonic acid amine (30) (rGalA1.6): Via general synthetic procedure 5 starting from D-GalA (2.0 g, 10.3 mmol). The Pd/C catalyst was filtered off on Celite and washed with MeOH. The filtrate was concentrated in vacuo to yield 30 (2.5 g, 90%) as a white solid. TLC (ACN/H₂O, 4/1, v/v): Rf=0.16; ¹H NMR (500 MHz, MeOD) δ 4.38-4.31 (m, 1H, H-2), 4.22 (d, J=1.8 Hz, 1H, H-5), 3.97 (dd, J=9.3, 1.8 Hz, 1H, H-4), 3.50 (dd, J=9.3, 1.4 Hz, 1H, H-3), 3.40 (dd, J=13.3, 10.6 Hz, 1H, H-1), 3.26-3.14 (m, 3H, H-1), 2.94 (s, 3H), 1.87-1.68 (m, 2H), 1.46-1.35 (m, 6H), 0.98-0.91 (m, 3H); ¹³C NMR (126 MHz, MeOD) δ 178.50 (COOH), 71.64 (C-4), 71.40 (C-3), 71.38 (C-5), 64.61 (C-2), 59.00 (C-1), 56.66, 40.20, 31.06, 25.94, 23.53, 22.11, 12.89; HRMS (m/z): [M+Na]⁺ calcd for C₁₃H₂₇NO₆, 316.17; found, 316.17365.

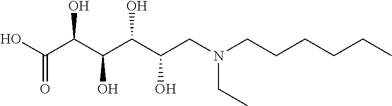 [SMILES: CCCCCCN(CC)C[C@H](O)[C@@H](O)[C@@H](O)[C@H](O)C(=O)O]

N-ethyl-N-hexyl-D-galacturonic acid amine (30b) (rGalA2.6): Starting from rGalA6 (200 mg) in or EtOH, palladium on activated carbon (10 wt % loading) was added under an argon atmosphere. The reaction mixture was bubbled through with hydrogen for 15 min. Acetaldehyde (1-3 eq) was added and was left stirring for 48/72/96 h at 35° C. The resulting mixture was purged of hydrogen by flushing with argon for 15 min. The Pd/C catalyst was filtered off on Celite and washed with MeOH and H₂O. The filtrate was concentrated in vacuo to yield 31 (231 mg, 96%) as an ivory solid. ¹H NMR (500 MHz, MeOD) δ 4.21 (dt, J=10.2, 2.6 Hz, 1H, H-2), 4.13 (d, J=2.1 Hz, 1H, H-5), 3.86 (dd, J=9.2, 2.1 Hz, 1H, H-4), 3.44 (dd, J=9.2, 1.8 Hz, 1H, H-3), 3.34-3.30 (m, 1H, H-1), 3.22 (dd, J=13.9, 6.8 Hz, 2H, H-1, CH₂), 3.12 (dd, J=15.2, 10.4 Hz, 3H, 2×CH₂), 1.70 (q, J=11.0 Hz, 2H), 1.35 (d, J=4.6 Hz, 6H), 1.32-1.25 (m, 3H), 0.97-0.84 (m, 3H). ¹³C NMR (126 MHz, MeOD) δ 178.22, 71.71, 71.64, 71.47, 64.95, 55.70, 31.13 (2×), 26.09 (2×), 23.54, 22.18, 12.93 (2×). MS (m/z): [M]⁺ calcd for C₁₄H₂₉NO₆, 308.20; found, 308.40

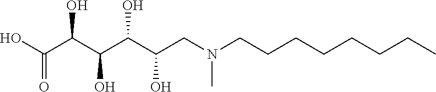 [SMILES: CCCCCCCCN(C)C[C@H](O)[C@@H](O)[C@@H](O)[C@H](O)C(=O)O]

N-methyl-N-octyl-D-galacturonic acid amine (31) (rGalA1.8): Via general synthetic procedure 5 starting from D-GalA (10.0 g, 51.5 mmol). The Pd/C catalyst was filtered off on Celite and washed with MeOH and H₂O. The filtrate was concentrated in vacuo to yield 31 (13.2 g, 88%) as an ivory solid. TLC (ACN/H₂O, 4/1, v/v): Rf=0.2; ¹H NMR (500 MHz, D₂O) δ 4.28 (d, J=12.2 Hz, 1H, H-2), 4.18 (t, J=1.2 Hz, 1H, H-5), 3.90 (dt, J=9.8, 1.2 Hz, 1H, H-4), 3.47 (dt, J=9.7, 1.2 Hz, 1H, H-3), 3.40 (d, J=11.1 Hz, 1H, H-1), 3.15 (dd, J=15.7, 12.3 Hz, 3H, H-1), 2.86 (s, 3H), 1.69 (dt, J=15.8, 7.5 Hz, 2H), 1.21 (td, J=8.2, 4.8 Hz, 10H), 0.83-0.77 (m, 3H); ¹³C NMR (126 MHz, D₂O) δ 179.15 (COOH), 71.34 (C-5), 71.21 (C-4), 70.48 (C-3), 64.02 (C-2), 57.96 (C-1), 50.43, 39.57, 30.96, 28.12, 28.11, 25.62, 23.19, 21.96, 13.37; HRMS (m/z): [M+H]⁺ calcd for C₁₅H₃₁NO₆, 322.22; found, 322.22274.

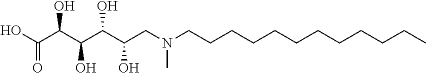 [SMILES: CCCCCCCCCCCCN(C)C[C@H](O)[C@@H](O)[C@@H](O)[C@H](O)C(=O)O]

N-methyl-N-dodecyl-D-galacturonic acid amine (32) (rGalA1.12): Via general synthetic procedure 5 starting from D-GalA (10.0 g, 51.5 mmol). The Pd/C catalyst was filtered off on Celite and washed with 3:1 EtOH/H₂O. The filtrate was concentrated in vacuo to yield 32 (15.1 g, 86%) as a white solid. TLC (ACN/H₂O, 4/1, v/v): Rf=0.3; ¹H NMR (500 MHz, EtOD) δ 4.36 (ddd, J=10.7, 2.8, 1.5 Hz, 1H, H-2), 4.21 (d, J=1.6 Hz, 1H, H-5), 3.98 (dd, J=9.7, 1.6 Hz, 1H, H-4), 3.53 (dd, J=9.6, 1.5 Hz, 1H, H-3), 3.44 (dd, J=13.2, 10.7 Hz, 1H, H-1), 3.22 (td, J=12.2, 9.6, 4.4 Hz, 3H, H-1), 2.95 (s, 3H), 1.79 (dp, J=24.0, 9.1, 7.6 Hz, 2H), 1.39-1.24 (m, 20H), 0.92-0.85 (m, 3H); ¹³C NMR (126 MHz, EtOD) δ 178.99 (COOH), 71.33 (C-5), 71.26 (C-4), 70.87 (C-3), 64.32 (C-2), 58.63 (C-1), 40.39, 31.65, 29.38, 29.35, 29.32, 29.29, 29.04, 28.99, 26.34, 23.73, 23.36, 22.34, 13.42; HRMS (m/z): [M+Na]⁺ calcd for C₁₉H₃₉NO₆, 400.27; found, 400.26864.

Compounds such as (31), (32), and (33) can also be described as the corresponding N-Methyl-N-Alkyl-D-Galactaric acid amines.

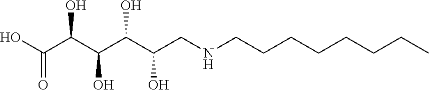 [SMILES: CCCCCCCCNC[C@H](O)[C@@H](O)[C@@H](O)[C@H](O)C(=O)O]

N-octyl-D-galacturonic acid amine (33) (rGalA8): Octylamine (2.7 g, 3.4 ml, 20.6 mmol) was added dropwise to a solution of D-GalA (2.0 g, 10.3 mmol) in methanol. The reaction mixture was stirred for 24 h at 20° C. The resulting mixture was cooled to 0° C. and NaBH4 (1.5 eq.) was added. After stirring for 2.5 h, the pH of the reaction mixture was lowered to 1 by dropwise addition of 6 M HCl. The resulting suspension was filtered, and the filtrate was concentrated in vacuo to yield 33 (2.9 g, 91%) as a white solid. TLC (ACN/H₂O, 4/1, v/v): Rf=0.2; ¹H NMR (500 MHz, D₂O) δ 4.56 (d, J=1.7 Hz, 1H, H-5), 4.23 (ddd, J=9.8, 3.3, 1.5 Hz, 1H, H-2), 4.06 (dd, J=9.7, 1.7 Hz, 1H, H-4), 3.63 (dd, J=9.8, 1.6 Hz, 1H, H-3), 3.30-3.17 (m, 2H, H-1), 3.13-3.07 (m, 2H), 1.71 (q, J=7.7, 7.3 Hz, 2H), 1.36-1.23 (m, 10H), 0.89-0.82 (m, 3H); ¹³C NMR (126 MHz, D₂O) δ 176.74 (COOH), 70.94 (C-4), 70.18 (C-5), 70.09 (C-3), 65.43 (C-2), 50.38 (C-1), 47.89, 30.97, 28.14, 28.11, 25.65, 25.29, 21.95, 13.36; HRMS (m/z): [M]⁺ calcd for C₁₄H₂₉NO₆, 308.20; found, 308.20733 (This is a reference compound).

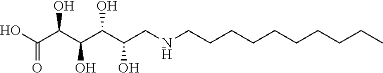 [SMILES: CCCCCCCCCCNC[C@H](O)[C@@H](O)[C@@H](O)[C@H](O)C(=O)O]

N-decyl-D-galacturonic acid amine (33b) (rGalA10): Via general synthetic procedure 5 starting from (1.0 g, 4.7 mmol). The Pd/C catalyst was filtered off on Celite and washed with MeOH and a basic solution. The filtrate was concentrated in vacuo to yield 33b (30 mg, 2%) as a yellow solid. TLC (ACN/H₂O, 4/1, v/v): Rf=0.63; ¹H NMR (500 MHz, METHANOL-D₄) δ 4.15 (d, J=5.1 Hz, 1H, H-2), 4.12 (d, J=2.2 Hz, 1H, H-5), 3.86 (d, J=8.8 Hz, 1H, H-4), 3.47 (d, J=9.1 Hz, 1H, H-3), 3.21-3.04 (m, 2H, H-1ab), 2.98 (q, J=6.9 Hz, 2H, CH₂), 1.67 (d, J=9.1 Hz, 2H), 1.39-1.20 (m, 14H), 0.88 (dd, J=7.7, 5.6 Hz, 3H). ¹³C NMR (126 MHz, D₂O) δ 178.13 (COOH), 71.99 (C-3), 71.67 (C-4), 71.50 (C-2), 66.06 (C-5), 50.93 (C-1), 47.31, 31.71, 29.27, 29.18, 29.08, 28.91, 26.29, 25.85, 22.38, 13.05. HRMS (m/z): [M+H]⁺ calcd for C₁₆H₃₃NO₆, 336.23; found, 336.23938.

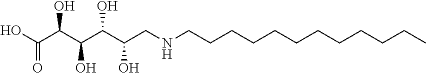 [SMILES: CCCCCCCCCCCCNC[C@H](O)[C@@H](O)[C@@H](O)[C@H](O)C(=O)O]

N-dodecyl-D-galacturonic acid amine (34) (rGalA12): N-dodecylamine (2 eq.) was added dropwise to a solution of from D-GalA (2.0 g, 10.3 mmol) in MeOH. The reaction mixture was stirred for 24 h at 20° C. The resulting mixture was cooled to 0° C. and NaBH4 (1.5 eq.) was added After stirring for 2.5 h, a yellow suspension had formed. The resulting suspension was filtered and washed with MeOH to yield 34 (0.1 g, 3.8%) as a yellow solid. TLC (ACN/H₂O, 4/1, v/v): Rf=0.13; HRMS (m/z): [M+Na]⁺ calcd for C₁₈H₃₇NO₆, 386.25; found, 386.25232 (This is a reference compound).

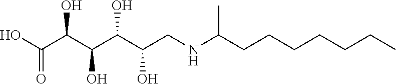 [SMILES: CCCCCCCC(C)NC[C@H](O)[C@@H](O)[C@@H](O)[C@H](O)C(=O)O]

N-(1-methyloctyl)-D-galacturonic acid amine (35): Via general synthetic procedure 5 starting from D-GalA (1.0 g, 5.2 mmol). The Pd/C catalyst was filtered off on Celite. The residue was washed with 3:1 EtOH/H₂O which was collected in a separate flask. The filtrate of the EtOH/H₂O wash was concentrated in vacuo to yield 35 (1.4 g, 83%) as a white solid. TLC (ACN/H₂O, 4/1, v/v): Rf=0.30; ¹H NMR (500 MHz, D₂O) δ 4.18 (d, J=1.5 Hz, 1H, H-5), 4.16 (ddd, J=9.7, 3.4, 1.5 Hz, 1H, H-2), 3.90 (dd, J=9.6, 1.6 Hz, 1H, H-4), 3.52 (dd, J=9.6, 1.6 Hz, 1H, H-3), 3.33-3.25 (m, 1H), 3.25-3.11 (m, 2H, H-1), 1.74-1.64 (m, 1H), 1.58-1.47 (m, 1H), 1.28-1.19 (m, 13H), 0.83-0.76 (m, 3H); ¹³C NMR (126 MHz, D₂O) δ 179.77 (COOH), 71.33 (C-5), 71.23 (C-4), 70.78 (C-3), 65.73 (C-2), 54.70, 47.38 (C-1), 32.42, 30.96, 28.31, 28.14, 24.54, 21.95, 15.21, 12.98; HRMS (m/z): [M+Na]⁺ calcd for C₁₅H₃₁NO₆, 344.20; found, 344.20392.

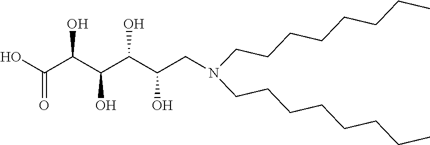 [SMILES: CCCCCCCCN(CCCCCCCC)C[C@H](O)[C@@H](O)[C@@H](O)[C@H](O)C(=O)O]

N,N-dioctyl-D-galacturonic acid amine (36): Via general synthetic procedure 5 starting from D-GalA (2.0 g, 10.3 mmol). The Pd/C catalyst was filtered off on Celite and washed with MeOH. The filtrate was concentrated in vacuo to yield 36 (3.8 g, 96%) as a yellow solid. TLC (ACN/H₂O, 4/1, v/v): Rf=0.11; ¹H NMR (500 MHz, MeOD) δ 4.29 (dt, J=10.2, 2.4 Hz, 1H, H-2), 4.19 (d, J=1.9 Hz, 1H, H-5), 3.94 (dd, J=9.2, 2.0 Hz, 1H, H-4), 3.49 (dd, J=9.3, 1.7 Hz, 1H, H-3), 3.43-3.34 (m, 1H, H-1), 3.26-3.16 (m, 5H, H-1), 1.81-1.73 (m, 4H), 1.40-1.27 (m, 10H), 0.96-0.90 (m, 6H); ¹³C NMR (126 MHz, MeOD) δ 178.26 (COOH), 71.61 (C-4), 71.57 (C-3), 71.38 (C-5), 64.81 (C-2), 56.69 (C-1), 31.50, 28.83, 26.33, 26.25, 25.88, 23.31, 22.28, 13.02; HRMS (m/z): [M+H]⁺ calcd for C₂₂H₄₅NO₆, 420.33; found, 420.33401.

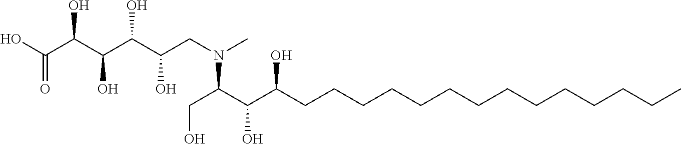 [SMILES: CCCCCCCCCCCCCC[C@H](O)[C@H](O)[C@@H](CO)N(C)C[C@H](O)[C@@H](O)[C@@H](O)[C@H](O)C(=O)O]

N-methylphytosphingosine-D-galacturonic acid amine (36b): Via general synthetic procedure 7 starting from D-GalA (2 g, 5.72 mmol). The Pd/C catalyst was filtered off on Celite and washed with MeOH and H₂O. The filtrate was concentrated in vacuo to form a mixture of 36b and starting materials. TLC (ACN/H₂O, 4/1, v/v): Rf=0.8; MS (m/z): [M+H]⁺ calcd for C₂₅H₅₁NO₉, 510.36; found, 510.28.

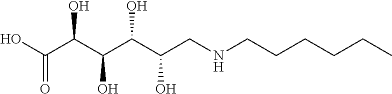 [SMILES: CCCCCCNC[C@H](O)[C@@H](O)[C@@H](O)[C@H](O)C(=O)O]

N-hexyl-D-galacturonic acid amine (37) (rGalA6): Via general synthetic procedure 5 starting from D-GalA (2.0 g, 10.3 mmol). The Pd/C catalyst was filtered off on Celite and washed with H₂O. The filtrate was concentrated in vacuo to yield 37 (2.4 g, 92%) as a white solid. ¹H NMR (500 MHz, D₂O) δ 4.21-4.12 (m, 2H, H-2, H-5), 3.89 (dt, J=9.6, 1.2 Hz, 1H, H-4), 3.51 (dt, J=9.7, 1.2 Hz, 1H, H-3), 3.25-3.11 (m, 2H, H-1), 3.08-2.99 (m, 2H), 1.70-1.58 (m, 2H), 1.32 (t, J=7.4 Hz, 2H), 1.25 (dt, J=7.1, 3.7 Hz, 4H), 0.81 (tt, J=7.0, 1.6 Hz, 3H); ¹³C NMR (126 MHz, D₂O) δ 179.57 (COOH), 71.32 (C-5), 71.21 (C-4), 70.73 (C-3), 65.60 (C-2), 50.43 (C-1), 47.86, 30.41, 25.32, 25.25, 21.69, 13.18; HRMS (m/z): [M+Na]⁺ calcd for C₁₂H₂₅NO₆, 380.17; found, 380.17554 (This is a reference compound).

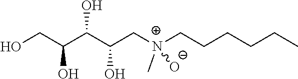 [SMILES: CCCCCC[N+](C)([O-])C[C@H](O)[C@@H](O)[C@@H](O)CO]

N-methylhexyl-L-arabinonamine oxide (38) (nAra1.6): Via general synthetic procedure 6 starting from 23 n-methylhexyl-L-arabinosamine (2 g, 8.021 mmol). Any remaining hydrogen peroxide was quenched with sodium thiosulphate and the mixture was concentrated in vacuo. The white solid was dissolved in methanol, filtrated and concentrated in vacuo to yield compound 23 (2.11 gr, 99%) as a white solid. TLC (ACN/H₂O (1% AA), 4/1, v/v): R_{f}=0.26. ¹H NMR (500 MHz, MeOD) δ 4.57 (ddt, J=9.3, 7.1, 1.9 Hz, 1H, H-3), 3.82 (ddd, J=11.0, 3.3, 1.6 Hz, 1H, H-5a), 3.75-3.64 (m, 1H, H-4), 3.55-3.49 (m, 2H, H5-b), 3.43-3.39 (m, 2H, H-1a, H-3), 3.37-3.28 (m, 3H, H-1b, CH₂), 3.20 (d, J=16.5 Hz, 3H), 1.93-1.79 (m, 2H), 1.43-1.36 (m, 6H), 0.97-0.92 (m, 3H). ¹³C NMR (126 MHz, MeOD) δ 73.04, 71.28, 70.81, 65.96, 64.01, 63.54, 55.03, 54.56, 32.23, 26.02, 22.98, 22.75, 22.13, 12.84 HRMS (m/z): [M+H]⁺ calculated for C₁₂H₂₇NO₅, 266.18892; found, 266.19595

Alternately Via general synthetic procedure 6 starting from 23 (2 g) The filtrate was concentrated in vacuo to yield 38 (2.11 g, 99%). as a white solid. TLC (ACN/H₂O, 4/1, v/v): Rf=0.20 ¹H NMR (500 MHz, MeOD) δ 4.57 (ddt, J=9.3, 7.2, 1.9 Hz, 1H), 3.82 (ddd, J=11.1, 3.3, 1.6 Hz, 1H), 3.72 (dddd, J=8.6, 7.0, 5.6, 3.3 Hz, 1H), 3.68-3.58 (m, 1H), 3.55-3.47 (m, 1H), 3.44-3.25 (m, 4H), 3.20 (d, J=16.5 Hz, 3H), 1.96-1.77 (m, 2H), 1.44-1.30 (m, 6H), 1.05-0.85 (m, 3H).

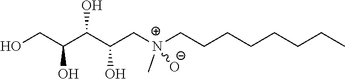 [SMILES: CCCCCCCC[N+](C)([O-])C[C@H](O)[C@@H](O)[C@@H](O)CO]

N-methyloctyl-L-arabinonamine oxide (39) (nAra1.8): Via general synthetic procedure 6 starting 24 n-methyloctyl-L-arabinosamine (2 g, 7.242 mmol). Any remaining hydrogen peroxide was quenched with sodium thiosulphate and the mixture was concentrated in vacuo. The white solid was dissolved in methanol, filtrated and concentrated in vacuo to yield compound 39 (2.082 gr, 98%) as a white solid. TLC (ACN/H₂O, 4/1, v/v): Rf=0.35; ¹H NMR (500 MHz, D₂O) δ 4.49 (dt, J=9.2, 1.6 Hz, 1H, H-2), 3.77 (ddd, J=11.9, 3.0, 1.4 Hz, 1H, H-5a), 3.68 (tdd, J=7.1, 3.7, 1.8 Hz, 2H, H-4), 3.59 (ddd, J=11.7, 5.9, 1.2 Hz, 1H, H-5b), 3.50-3.43 (m, 1H, H-1a), 3.41 (m, 1H, H-3), 3.37-3.26 (m, 3H, H-1b, CH₂), 3.13 (d, J=5.4 Hz, 3H), 1.80-1.61 (m, 2H), 1.32-1.17 (m, 10H), 0.84-0.78 (m, 3H). ¹³C NMR (126 MHz, D₂O) δ 72.31, 70.56, 70.49, 70.32, 65.49, 63.17, 55.43, 31.32, 28.61, 26.01, 23.01, 22.80, 22.22, 13.60. HRMS (m/z): [M+H]⁺ calculated for C₁₄H₃₁NO₅, 294.22022; found, 294.22853

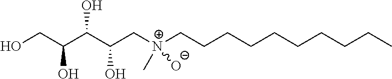 [SMILES: CCCCCCCCCC[N+](C)([O-])C[C@H](O)[C@@H](O)[C@@H](O)CO]

N-methyldecyl-L-arabinonamine oxide (40) (nAra1.10): Via general synthetic procedure 6 starting n-methyldecyl-L-arabinosamine (200 mg). MS was observed (MS+H+) 322.47.

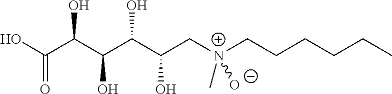 [SMILES: CCCCCC[N+](C)([O-])C[C@H](O)[C@@H](O)[C@@H](O)[C@H](O)C(=O)O]

N-methyl-N-hexyl-D-galacturonic acid amine oxide (41) (nGalA1.6): Via general synthetic procedure 6 starting from N-methyl-N-hexyl-D-galacturonic acid amine 30 (2 g, 6.818 mmol). Any remaining hydrogen peroxide was quenched with sodium thiosulphate and the mixture was concentrated in vacuo. The white solid was dissolved in methanol, filtrated and concentrated in vacuo to yield compound 41 (1.94 gr, 92%) as a white solid. TLC (ACN/H₂O, 4/1, v/v): Rf=0.19; ¹H NMR (500 MHz, MeOD) δ 4.64-4.56 (m, 1H, H-2), 4.23 (d, J=1.5 Hz, 1H, H-5), 3.99 (ddd, J=9.6, 4.2, 1.7 Hz, 1H, H-4), 3.61-3.49 (m, 2H, H-1a, H-3), 3.46-3.28 (m, 3H, H-1b), 3.22 (d, J=13.6 Hz, 3H), 1.96-1.78 (m, 2H), 1.39 (h, J=3.2 Hz, 6H), 0.98-0.91 (m, 3H); ¹³C NMR (126 MHz, MeOD) δ 179.01 (COOH), 72.66 (C-3), 71.57 (C-4), 71.47 (C-5), 70.49 (C-1), 69.98, 65.49 (C-2), 54.60, 31.26, 26.06, 22.79, 22.17, 12.91; HRMS (m/z): [M+H]⁺ calcd for C₁₃H₂₇NO₇, 310.18; found, 310.18752.

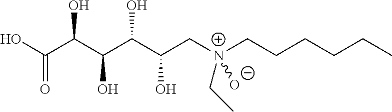 [SMILES: CCCCCC[N+]([O-])(CC)C[C@H](O)[C@@H](O)[C@@H](O)[C@H](O)C(=O)O]

N-ethyl-N-hexyl-D-galacturonic acid amine oxide (41b) (nGalA2.6): Via general synthetic procedure 6 starting from N-ethyl-N-hexyl-D-galacturonic acid amine 30b (220 mg). Compound 41b was observed, almost complete conversion. MS (m/z): [M+H]⁺ calcd for C₁₄H₂₉NO₇, 324.19; found, 324.47

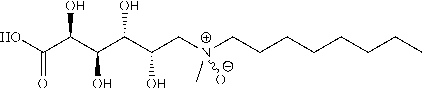 [SMILES: CCCCCCCC[N+](C)([O-])C[C@H](O)[C@@H](O)[C@@H](O)[C@H](O)C(=O)O]

N-methyl-N-octyl-D-galacturonic acid amine oxide (42) (nGalA1.8): Via general synthetic procedure 6 starting from N-methyl-N-octyl-D-galacturonic acid amine 31 (2 g, 6.818 mmol). Any remaining hydrogen peroxide was quenched with sodium thiosulphate and the mixture was concentrated in vacuo. The white solid was dissolved in methanol, filtrated and concentrated in vacuo to yield compound 42 (1.70 gr, 81%) as a white solid. TLC (ACN/H₂O, 4/1, v/v): Rf=0.19; ¹H NMR (500 MHz, D₂O) δ 4.58 (ddt, J=9.3, 4.7, 1.6 Hz, 1H, H-2), 4.26 (t, J=1.4 Hz, 1H, H-5), 3.99 (dt, J=9.7, 1.4 Hz, 1H, H-4), 3.62-3.52 (m, 2H, H-1a, H-3), 3.50-3.30 (m, 3H, H-1b), 3.22 (d, J=4.1 Hz, 3H), 1.85-1.72 (m, 2H), 1.38-1.22 (m, 10H), 0.91-0.84 (m, 3H); ¹³C NMR 126 MHz, D₂O) δ 180.08 (COOH), 71.97 (C-3), 71.34 (C-5), 71.09 (C-4), 70.48 (C-1), 69.70, 65.37 (C-2), 55.08, 30.98, 28.29, 28.21, 25.75, 22.66, 21.97, 13.40; HRMS (m/z): [M+H]P calcd for C₁₅H₃₁NO₇, 338.21; found, 338.21978.

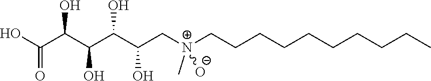 [SMILES: CCCCCCCCCC[N+](C)([O-])C[C@H](O)[C@@H](O)[C@@H](O)[C@H](O)C(=O)O]

N-methyl-N-octyl-D-galacturonic acid amine oxide (42b) (nGalA1.10): Via general synthetic procedure 6 starting from N-methyl-N-decyl-D-galacturonic acid amine 25b (1 g, 2.861 mmol). Any remaining hydrogen peroxide was quenched with sodium thiosulphate and the mixture was concentrated in vacuo. The white solid was dissolved in methanol, filtrated and concentrated in vacuo to yield compound 42b (0.38 gr, 36%) as a white solid. TLC (ACN/H₂O (1% AA), 4/1, v/v): Rf=0.33 1H NMR (500 MHz, METHANOL-D₄) δ 4.55 (dd, J=10.6, 8.8 Hz, 1H), 4.22-4.14 (m, 1H), 3.58-3.50 (m, 1H), 3.49-3.42 (m, 1H), 3.41-3.33 (m, 2H), 3.21 (d, J=8.9 Hz, 3H), 1.83 (dtd, J=23.4, 12.1, 6.5 Hz, 2H), 1.44-1.16 (m, 14H), 0.87 (t, J=6.8 Hz, 3H). 13C NMR (126 MHz, METHANOL-D₄) δ 178.48 (COOH), 72.77 (C3), 71.69 (C4), 71.36 (C5), 71.23 (C1), 69.94, 65.61 (C2), 55.20, 35.53, 31.86, 29.33, 29.20, 26.41, 25.52, 23.07, 22.47, 13.14 HRMS (m/z): [M+H]⁺ calculated for C₁₇H₃₅NO₇, 366.24135; found, 366.24908.

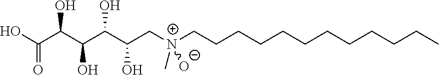 [SMILES: CCCCCCCCCCCC[N+](C)([O-])C[C@H](O)[C@@H](O)[C@@H](O)[C@H](O)C(=O)O]

N-methyl-N-dodecyl-D-galacturonic acid amine oxide (43) (nGalA1.12): Via general synthetic procedure 6 starting from N-methyl-N-dodecyl-D-galacturonic acid amine 32 (2 g, 5.298 mmol). Any remaining hydrogen peroxide was quenched with sodium thiosulphate and the mixture was concentrated in vacuo. The white solid was dissolved in methanol, filtrated and concentrated in vacuo to yield compound 43 (1.52 g, 73%) as a white solid. TLC (ACN/H₂O, 4/1, v/v): Rf=0.23; ¹H NMR (500 MHz, MeOD) δ 4.59 (tt, J=9.1, 1.7 Hz, 1H, H-2), 4.22 (t, J=1.2 Hz, 1H, H-5), 3.98 (ddd, J=9.4, 6.0, 1.7 Hz, 1H, H-4), 3.60-3.48 (m, 2H, H-1a, H-3), 3.46-3.34 (m, 3H, H-1 b), 3.21 (d, J=13.5 Hz, 3H), 1.96-1.78 (m, 2H), 1.44-1.26 (m, 18H), 0.92 (t, J=6.9 Hz, 3H); ¹³C NMR (126 MHz, MeOD) δ 178.87 (COOH), 72.63 (C-3), 71.58 (C-4), 71.39 (C-5), 71.23 (C-1), 69.87, 65.58 (C-2), 55.47, 31.66, 29.33, 29.26, 29.21, 29.06, 29.05, 26.41, 26.38, 23.07, 22.32, 13.03; HRMS (m/z): [M+H]⁺ calcd for C₁₉H₃₉NO₇, 394.27; found, 394.27820.

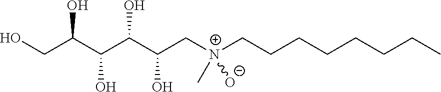 [SMILES: CCCCCCCC[N+](C)([O-])C[C@H](O)[C@@H](O)[C@H](O)[C@H](O)CO]

1-(N-methyl-N-octyl)-D-glucosamine oxide (44) (nGlc1.8): Via general synthetic procedure 6 starting from N-methyloctyl-D-glucosamine (3.4 g). After the reaction was completed (as analysed by MS) a small amount was taken out of the reaction flask, dried by air at 50° C., and measured with NMR. TLC (ACN/H₂O, 4/1, v/v): Rf=0.35; HRMS (m/z): [M+H]⁺ calcd for C₁₅H₃₃NO₆, 324.23; found, 324.40. ¹H NMR (500 MHz, METHANOL-D₄) δ 8.52 (s, 1H), 4.39 (dq, 1H, H-2), 3.78-3.75 (m, 1H, H-3), 3.74 (d, 1H, H-6A), 3.70-3.67 (m, 1H, H-4), 3.66-3.64 (m, 1H, H-5), 3.61 (d, 1H, H-6B), 3.44 (dt, 2H, H-1), 3.39-3.30 (m, 1H), 3.18-3.11 (m, 3H, H-7), 1.87-1.74 (m, 2H), 1.40-1.22 (m, 10H), 0.91-0.84 (m, 3H). 13C NMR (126 MHz, METHANOL-D₄) δ 169.14, 71.65, 71.63, 71.61 (C-5), 71.37 (C-4), 70.75, 70.43 (C-3), 69.77 (C-1), 68.58 (C-2), 63.31 (C-6), 61.50, 55.32 (C-7A), 54.46 (C-7B), 48.19, 48.03, 47.86, 47.68, 47.51, 47.34, 47.17, 31.58, 29.07, 28.93, 26.44, 26.42, 23.13, 22.85, 22.35, 13.09.

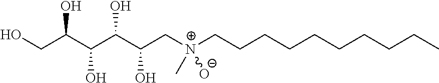 [SMILES: CCCCCCCCCC[N+](C)([O-])C[C@H](O)[C@@H](O)[C@H](O)[C@H](O)CO]

1-(N-methyl-N-decyl)-D-glucosamine oxide (45) (nGlc1.10): Via general synthetic procedure 6 starting from N-methyldecyl-D-glucosamine (483 mg), H₂O₂ (0.124 mL, 1 eq), NaOH (3 eq) in water (100 mL), while monitoring the pH at 14 and temperature at 50° C. MS was collected after 12 h, 132 h and 204 h. After 134 h 50 mL water was added to increase solubility. Extra H₂O₂ was added (5 eq.) to complete the reaction. After the reaction was completed (as analysed by MS) a small amount was taken out of the reaction flask and dried by air at 50° C., and measured with NMR. ¹H NMR (500 MHz, METHANOL-D₄) δ 4.39 (m, 1H, H-2), 3.76 (m, 1H, H-6A), 3.75 (m, 1H, H-3), 3.67 (m, 1H, H-4), 3.67 (m, 1H, H-4), 3.65 (m, 1H, H-5), 3.60 (m, 1H, H-6B), 3.43 (m, 1H, H-1), 3.36-3.30 (m, 1H), 3.17 (d, J=1.7 Hz, 2H), 3.16-3.12 (m, 1H), 1.86-1.74 (m, 1H), 1.40-1.20 (m, 14H), 0.87 (td, J=7.1, 2.2 Hz, 3H). ¹³C NMR (126 MHz, METHANOL-D₄) δ 71.56 (C-5), 71.34 (C-4), 70.61 (C-3), 69.72 (C-1), 68.49 (C-2), 63.42 (C-6), 61.52, 55.23, 54.56, 31.70, 29.28, 29.25, 29.07, 26.40, 22.38, 13.07.

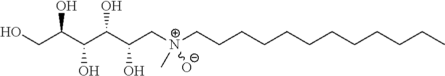 [SMILES: CCCCCCCCCCCC[N+](C)([O-])C[C@H](O)[C@@H](O)[C@H](O)[C@H](O)CO]

1-(N-methyl-N-dodecyl)-D-glucosamine oxide (46) (nGlc1.12): Via general synthetic procedure 6 starting from N-methyldodecyl-D-glucosamine (250 mg, 0.687 mmol), Any remaining hydrogen peroxide was quenched with sodium thiosulphate and the mixture was concentrated in vacuo. The white solid was dissolved in methanol, filtrated and concentrated in vacuo to yield compound 46 (130 mg, 80%). TLC (ACN/H₂O, 4/1, v/v): Rf=0.45 1H NMR (500 MHz, METHANOL-D₄) δ 4.39 (dt, J=6.8, 4.3 Hz, 1H, H-2), 3.76 (dd, J=3.8, 1.4 Hz, 1H, H-3), 3.74 (d, J=2.7 Hz, 1H, H-6a), 3.69-3.65 (m, 2H, H-5, H-4), 3.62 (t, J=5.2 Hz, 1H, H-6b), 3.47-3.41 (m, 2H, H-1), 3.39-3.28 (m, 2H, CH₂), 315 (d, J=13.1 Hz, 3H, CH₃), 1.85-1.78 (m, 2H), 1.27 (d, J=9.0 Hz, 18H), 0.92-0.75 (m, 3H). 13C NMR (126 MHz, METHANOL-D₄) δ 71.68 (C4), 71.38 (C5), 70.39 (C3), 69.69 (CH2), 69.00 (C1), 68.58 (C2), 63.25 (C6), 54.87 (CH3), 68.58 31.73, 29.41, 29.33, 29.26, 29.13, 29.11, 26.43, 23.13, 22.85, 22.40, 13.38. HRMS (m/z): [M+H]⁺ calcd for C₁₉H₄₁NO₆, 380.29; found, 380.30044

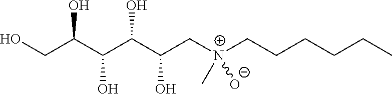 [SMILES: CCCCCC[N+](C)([O-])C[C@H](O)[C@@H](O)[C@H](O)[C@H](O)CO]

1-(N-methyl-N-hexyl)-D-glucosamine oxide (47) (nGlc1.6): Via general synthetic procedure 6 starting from N-methylhexyl-D-glucosamine (3.0 g), After the reaction was completed (as analysed by MS) a small amount was taken out of the reaction flask, dried by air at 50° C., and measured with NMR. ¹H NMR (500 MHz, METHANOL-D₄) δ 8.53 (s, 1H), 4.42 (m, 1H, H-2), 3.80-3.77 (m, 1H, H-3), 3.76 (m, 1H, H-6A), 3.73 (ddd, 1H, H-4), 3.69 (ddd, 1H, H-5), 3.64 (m, 1H, H-6B), 3.53 (m, 2H, H-1), 3.47-3.35 (m, 2H), 3.19 (d, 3H, H-7), 1.89-1.75 (m, 2H), 1.41-1.21 (m, 6H), 0.89 (dtd, 3H). ¹³C NMR (126 MHz, METHANOL-D₄) δ 169.27, 71.70, 71.61 (C-5), 71.31 (C-4), 70.68, 70.35 (C-3), 69.13 (C-1), 68.65 (C-2), 63.39, 63.31, 63.20 (C-6), 61.58, 55.85 (C-7A), 54.44 (C-7B), 48.31, 48.14, 47.97, 47.80, 47.63, 47.46, 47.29, 31.55, 31.30, 26.12, 26.08, 23.20, 22.78, 22.34, 22.25, 13.13, 13.06.

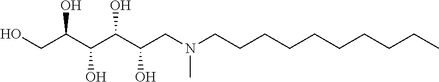 [SMILES: CCCCCCCCCCN(C)C[C@H](O)[C@@H](O)[C@H](O)[C@H](O)CO]

1-(N-methyl-N-octyl)-D-glucosamine (48) (rGlc1.8): Via general synthetic procedure 7 starting from D-glc (2.000 g, 11.10 mmol). The Pd/C catalyst was filtered off on Celite and washed with EtOH. The filtrate was dried in vacuo, yielding rGlc1.8 (3.4 g, 100%) as a white solid. NMR showed that a very little amount of glucose is still present (˜1%). ¹H NMR (500 MHz, METHANOL-D₄) δ 3.86 (dt, 1H, H-2), 3.76 (dd, 1H, H-6A), 3.72 (dd, 1H, H-3), 3.68 (ddd, 1H, H-4), 3.62-3.60 (m, 1H, H-5), 3.60-3.57 (m, 1H, H-6B), 2.54 (dd, 1H, H-1), 2.50-2.37 (m, 2H), 2.29 (s, 3H, H-7), 1.48 (q, 2H), 1.35-1.21 (m, 10H), 0.91-0.82 (m, 3H). ¹³C NMR (126 MHz, METHANOL-D₄) δ 72.00 (C-5), 71.65 (C-3 and C-4), 70.37 (C-2), 63.59 (C-6), 60.18 (C-1), 58.25, 48.19, 48.02, 47.85, 47.68, 47.51, 47.34, 47.17, 41.65 (C-7), 31.68, 29.33, 29.09, 27.23, 26.55, 22.39, 13.10.

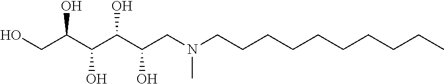 [SMILES: CCCCCCCCCCN(C)C[C@H](O)[C@@H](O)[C@H](O)[C@H](O)CO]

1-(N-methyl-N-decyl)-D-glucosamine (49) (rGlc1.10): Via general synthetic procedure 7 starting from D-glc (1.997 g, 11.08 mmol). The Pd/C catalyst was filtered off on Celite and washed with MeOH and EtOH. The filtrate was washed with heptane, however the product also dissolved in heptane. Column chromatography was used to isolate the product. Product got stuck on the column, so the column was flushed and washed with pure MeOH, resulting in two batches (flush and wash). The MeOH was dried off, and to get rid of impurities ethyl acetate was added. MS (MS flush1 and MS wash1) was collected. Evaporation of ethyl acetate yielded a very low amount of product. The two batches were washed again with DCM/MeOH (10%) and MS (MS flush2 and wash2) was collected. Also NMR was collected. This yielded rGlc1.10 as a brownish (batch 1) and a yellow/off white (batch 2) paste (1468 mg, 39%). ¹H NMR (500 MHz, MeOD) δ 3.86 (d, J=6.5 Hz, OH, H-2), 3.79 (dd, J=2.9, 1.9 Hz, OH, H-6B), 3.72 (dd, J=4.6, 1.9 Hz, 1H, H-3), 3.70-3.66 (m, OH, H-4), 3.62-3.57 (m, 2H, H-6A & H-5), 2.54 (dd, J=6.0, 2.7 Hz, 1H, H-1), 2.44 (dd, J=7.7, 5.7 Hz, 1H), 2.29 (s, 3H, H-7), 1.53-1.44 (m, 2H), 1.28 (dd, J=10.2, 4.1 Hz, 14H), 0.87 (t, J=6.9 Hz, 3H). ¹³C NMR (126 MHz, METHANOL-D₄) δ 72.00 (C-5), 71.66 (C-3 & C-4), 70.38 (C-2), 63.59 (C-6), 60.19 (C-1), 58.26, 41.65 (C-7), 31.72, 29.41, 29.37, 29.35, 29.29, 29.12, 27.22, 26.56, 22.39, 13.09.

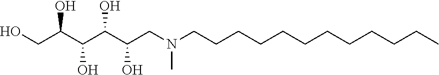 [SMILES: CCCCCCCCCCCCN(C)C[C@H](O)[C@@H](O)[C@H](O)[C@H](O)CO]

1-(N-methyl-N-dodecyl)-D-glucosamine (50) (rGlc1.12): Via general synthetic procedure 7 starting from D-glc (1.0 g, 5.2 mmol). The Pd/C catalyst was filtered off on Celite and washed with MeOH and EtOH. The filtrate was concentrated in vacuo to yield 50 (620 mg, 1.7 mmol). TLC (ACN/H₂O, 4/1, v/v): Rf=0.4. 1H NMR (500 MHz, METHANOL-D₄) δ 3.88-3.83 (m, 1H. H-1), 3.76 (dd, J=11.2, 3.6 Hz, 1H, H-6a), 3.72 (dd, J=4.6, 1.9 Hz, 1H, H-3), 3.70-3.65 (in, 1H, H-5), 3.64-3.55 (m, 2H, H-4, H-6b), 2.55 (dd, J=5.9, 2.5 Hz, 2H, H-1), 2.50-2.39 (m, 2H), 2.30 (s, 3H), 1.52-1.44 (m, 2H), 1.27 (d, J=4.7 Hz, 18H), 0.87 (t, J=7.0 Hz, 3H). 13C NMR (126 MHz, METHANOL-D₄) δ 71.99, 71.65 (2×), 70.36, 63.59, 60.17, 58.25, 41.64, 31.74, 29.42 (3×), 29.35, 29.14, 27.81, 27.21, 26.54, 22.39, 13.09. HRMS (m/z): [M+H]⁺ calcd for C₁₉H₄₁NO₅, 364.30; found, 364.30438

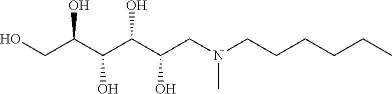 [SMILES: CCCCCCN(C)C[C@H](O)[C@@H](O)[C@H](O)[C@H](O)CO]

1-(N-methyl-N-hexyl)-D-glucosamine (51) (rGlc1.6): Via general synthetic procedure 7 starting from D-glc (2.001 g, 11.11 mmol). The Pd/C catalyst was filtered off on Celite and washed with MeOH and EtOH. the filtrate was washed with heptane, and subsequently dissolved in DCM and heptane. DCM was dried to air at rt and white crystals were formed. The heptane layer was discarded and the white crystals were dissolved in methanol, The product was dried in vacuo yielding rGlc1.6 (˜3.0 g, 99%) as a clear oily substance. ¹H NMR (500 MHz, METHANOL-D₄) δ 3.86 (dt, 1H, H-2), 3.76 (dd, 1H, H-6A), 3.72 (dd, 1H, H-3), 3.69 (ddd, 1H, H-4), 3.62-3.60 (m, 1H, H-5), 3.60-3.57 (m, 1H, H-6B), 2.54 (dd, 1H, H-1), 2.50-2.34 (m, 2H), 2.29 (s, 3H, H-7), 1.54-1.41 (m, 2H), 1.29 (dp, 6H), 0.94-0.84 (m, 3H). ¹³C NMR (126 MHz, METHANOL-D₄) δ 72.01 (C-5), 71.65 (C-3 and C-4), 70.39 (C-2), 63.60 (C-6), 60.20 (C-1), 58.27, 48.24, 48.07, 47.90, 47.73, 47.56, 47.39, 47.22, 41.69 (C-7), 31.62, 26.93, 26.52, 22.38, 13.11.

### Varying Reaction Conditions

For the following reaction (see scheme below) some reaction conditions were varied as depicted in the table below.

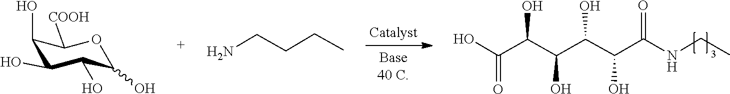

| Entry | Catalyst | Base | Product formation |
|---|---|---|---|
| 1 | Au-Ti-O₂ | K₂CO₃ | + |
| 2 | Au-Ti-O₂ | Cs₂CO₃ | + |
| 3 | Au-Ti-O₂ | KOH | − |
| 4 | Au-Ti-O₂ | triethylamine | ++ |
| 5 | Au-Ti-O₂ | diisopropylethylamine | ++ |
| 6 | Au-C | triethylamine | + |
| 7 | Au-ZnO | triethylamine | ++ |
| 8 | Au-Al₂O₃ | triethylamine | + |
| 9 | Pt-Al₂O₃ | triethylamine | ++ |
| 8 + 9 | Mix | triethylamine | ++ |

Additional Galacturonic acid examples are as follows—Au/TiO₂ (0.07 eq. Au) catalyzed oxidative amidation reaction optimalization, full conversion in closed environment (16-22 h, 200 mg)

| Entry | Conditions | Yield |
|---|---|---|
| 1 | 40° C., 2 eq. TEA, 2 eq. butylamine | 21% |
| 2 | 50° C., 2 eq. TEA, 2 eq. butylamine | 25% |
| 3 | 60° C., 2 eq. TEA, 2 eq. butylamine | 2% |
| 4 | 50° C., 2 eq. Cs₂CO₃, 2 eq. butylamine | 6% |
| 5 | 50° C., 3 eq. TEA, 2 eq. butylamine | 11% |
| 6 | 50° C., 3 eq. TEA, 1 eq. butylamine | <1% |
| 7 | 40° C., 2 eq. K₂CO₃, 2 eq. butylamine | 11% |
| 8 | 50° C., 1 eq. TEA, 2 eq. butylamine | 5% |
| 9 | 35° C., 2 eq TEA, 2 eq octylamine | 20% |
| 10 | 35° C., 2 eq TEA, 2 eq butylamine + | 20% |
| | intern standard | |
| 11 | 35° C., 2 eq TEA, 5 eq octylamine | 20% |
| 12 | 35° C., 0 eq TEA, 5 eq octylamine | 17% |
| 13 | 35° C., 2 eq TEA, 2 eq octylamine | 0% |
| | (Au later) | |
| 14 | RT, 2 eq TEA, 2 eq octylamine | 5% |
| | (Au later) | |
| 15 | 35° C., 2 eq TEA, 2 eq octylamine | 10% |
| | (dropwise) | |
| 16 | 60° C., 2 eq TEA, 2 eq octylamine | 40% |
| 17 | 80° C., 2 eq TEA, 2 eq octylamine | 40% |

For the following reaction (see scheme below) some reaction conditions were varied as depicted in the table below. (for rGalA1.8 (31) and rGalA8(33) as reference)

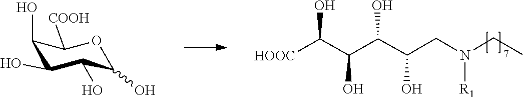

| Entry | Conditions step 1 | Conditions step 2 | Product | Yield |
|---|---|---|---|---|
| 1 | octylamine (2 eq.), | N.A. | rGalA8: | 91% |
| | NaBH₄, 0º C.-rt, MeOH | | R₁ = H | |
| 2 | octylamine (1 eq.), | N.A. | rGalA8: | 26% |
| | Pd/C, H₂, rt, MeOH | | R₁ = H | |
| 3 | N,N-methyloctylamine (2 eq.), | N.A. | rGalA1.8: | 19% |
| | NaBH₄, 0º C.-rt, MeOH | | R₁ = Me | |
| 4 | N,N-methyloctylamine (1 eq.), | N.A. | rGalA1.8: | 88% |
| | Pd/C, H₂, rt, MeOH | | R₁ = Me | |
| 5 | octylamine (1 eq.), Pd/C, H₂, rt, | formaldehyde (3.0 | rGalA1.8: | 97% |
| | MeOH | eq.) | R₁ = Me | |
| 6 | octylamine (1 eq.), Pd/C, H₂, | formaldehyde (3.0 | rGalA1.8: | 88% |
| | 35º C., MeOH | eq.) | R₁ = Me | |
| 7 | octylamine (1 eq.), Pd/C, H₂, | formaldehyde (3.0 | rGalA1.8: | 99% |
| | 35° C., EtOH | eq.) | R₁ = Me | |

In case of the one-pot reaction, the following scheme applies:

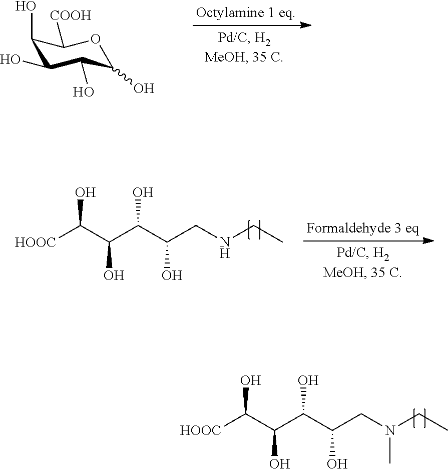

### Example 2—Characterisation of Compounds

### 2.1.1 Solubility

Solubility in water was determined by preparing mixtures of an excess of compound dispersed in 4 ml demi water using a vortex at maximum speed for 1 min. Amounts of 100 to 300 mg were generally used for compounds with chains of 10 carbon atoms or less, whereas 10 to 30 mg were generally used for compounds with longer chains. 1 ml of the mixture was concentrated in vacuo to yield a solid. The mass of the solid was determined in mg to calculate the solubility in percentage by dividing by ten.

### 2.1.2 Foaming

Foaming abilities were evaluated at room temperature by shaking 1% compound solutions (2 ml) of pH 3, 7, and 12 by hand for 10 s. The pH of each solution was assessed with a pH meter at room temperature. For compounds with a solubility of >1%, solutions at maximum solubility were used. The foam heights at the initial time were compared to one another after which the foaming abilities were classified according to no foam formation (−), low foam formation (+), moderate foam formation (++), and strong foam formation (+++).

Foaming power was also tested at room temperature with 5 mL of 0.4% aq. surfactant (20 mg) solution in a 50 mL measuring cylinder. After performing the Ultra-Turrax (a registered trademark for a high-performance dispersing instrument) for 20 seconds (8000 rpm), the initial height of the foam was measured for foam height. (volume in mL). Every time step of, 1, 3, 5, 10, 30, 60 minutes was noted. In addition, after 24 hours the stability was checked. Particularly, foaming power and stability of the surfactants were performed using an IKA Ultra-Turrax T25 with an 18 G dispersing element. At room temperature, an aqueous solution of 5 mL 0.4% surfactant in a 50 mL measuring cylinder (2.20 cm diameter) was mixed with the Ultra-Turrax for 20 seconds at 8000 rpm. The foam height (volume in mL) was denoted for 0, 1, 3, 5, 10, 30, 60 min and 24 hours. In addition, after 24 hours the stability was checked.

### 2.1.3 Surface Tension

Surface tension measurements were performed with a Biolin Scientific Sigma701 automatic tension meter equipped with a Wilhelmy plate made of platinum (±0.5 mN/m). At room temperature and a pH of 7, different concentrations of aqueous solutions (0.01%-max. 12.5%; 4 ml) of synthesized compounds and of commercially available surfactants (SLES, which is sodium laureth sulfate, and SCG, which is sodium cocoyl glutamate) were measured continuously (30 sec) (‘continuous Wilhelmy plate’ method) using small plastic petri dishes. The number of experimental points is related to the compound's/surfactant's solubility and available quantity. All compound/surfactant solutions were allowed to stand 12 h before being measured. The platinum plate was thoroughly cleaned with demi water and flame-dried before each measurement. The CMC of each compound/surfactant was determined at the minimum of a breakpoint of a conventional plot of surface tension versus compound/surfactant concentration. DCMC was obtained by acquiring the surface tension at the CMC. The pC₂₀ value was determined by taking the minus log of the concentration compound/surfactant at a surface tension of 52 mN/m.

### 2.1.4 Skin Irritation Properties

Zein solubilisation was measured to determine the irritation of the surfactants. Zein is a simple protein found in corn. It is a very simple mimic of skin protein, the more solubilised by your material the harsher the potential to irritate and dry the skin. This method is a quick screen and provides indicative data. The procedure is 1) Weight 2-2.5 ml Eppendorf's on a 4 dp balance. 2) Weight out as accurately as possible 70 mg of Zein. 3) Add to the Eppendorf and reweigh. 4) Make your surfactant dilutions. 5) Add 1 ml of the surfactant dilution to the Eppendorf. 6) Place this on a rotary mixer for 20 mins at 20 rpm at RT. 7) Remove and centrifuge for 4 mins at highest speed available on a micro centrifuge 8) Discard the supernatant making sure as much of it possible is removed. 9) Repeat with water, centrifuge and discard supernatant. 10) With the Eppendorf lid open, place at 50° C. overnight to dry (48 hours required). 11) Re-weigh the next day. 12) Calculate the amount of Zein solubilised. The obtained data allows comparison of surfactants and their harshness to skin. Decisions for in vivo testing can be based on good Zein results.

### 2.1.5 Biodegradability

Biodegradability of compounds was assessed. The compound is added to an inoculate of activated sludge from a municipal wastewater treatment plant (WWTP). The experiment relies on the basic principles of the 301A DOC Die-Away Test described in the OECD guidelines for readily biodegradable substances. A small adjustment was made where the measuring is not done in conical flasks but, instead, in serum vials closed with butylrubber stoppers. This allowed measurement of the evolution of CO₂ and O₂ over time. In general, the experiment involves the experimental incubation (inoculum and compound), along with a blank control (inoculum no compound), a positive control (inoculum and sodium acetate), and controls for abiotic degradation (compound in medium), adsorption (compound with sterilized inoculum), and toxicity (inoculum with compound and sodium acetate).

Activated sludge was obtained from the wastewater treatment facility in Weurt, The Netherlands, from an aerated sludge basin. The basin was operated at 20.7° C. and contained 2.29 g/L total suspended solids (TSS) and 1.04 g/L dissolved oxygen (DO). The fresh sludge was conditioned overnight by bubbling with air at room temperature. Before incubation, the sludge was washed once in mineral medium containing per L ddH₂O: 85 mg KH₂PO₄, 217.5 mg K₂HPO₄, 334 mg Na₂HPO₄*2H₂O, 5 mg NH₄Cl, 27.5 mg CaCl₂, 22.5 mg MgSO₄*7H₂O, 0.25 mg FeCl₃*6H₂O, and 0.4 mg 2Na-EDTA.

Incubations were prepared containing approximately 70 mg/L surfactant and 3 ml sludge in a final volume of 30 ml mineral medium in a borosilicate serum vial crimp-sealed with a butyl rubber stopper. In no sludge controls, the sludge was replaced with additional medium. In inactive controls, autoclaved (20 min at 120° C.) sludge was used. In toxicity controls, approximately 135 mg/L sodium acetate was added alongside sludge and surfactant. In no substrate controls, both acetate and surfactant were omitted. In acetate only controls, 135 mg/L sodium acetate was added as the sole carbon source. All incubations were kept at 20° C. shaking 300 rpm throughout the duration of the experiment. 1 ml samples were taken weekly for surfactant quantification and centrifuged 5 min at 20.000×g. The supernatant was diluted in a mixture of 20% supernatant, 40% acetonitrile, and 40% methanol and stored at −70° C. until analysis. Calibration curves of the surfactant were prepared by spiking surfactant in the supernatant of the substrate-free controls to account for matrix effects. Measurements were taken by on an Agilent HPLC/MS-QToF in positive ionization mode. CO₂ was analyzed by measuring headspace gas on an Agilent 8890A/5977B GC-MS equipped with an Agilent 6 FT Porapak Q 80/10 column.

### 2.2 Results

Surface tension, solubility and foam creation of surfactants are important properties. The non-ionic L-Ara based surfactants with a secondary amide appear to not create foam. 01, 02, and 05 are soluble in water, but do not reach a stable surface tension value in their maximal concentration. This means that no micelles were formed. Although 03 and 04 are poorly soluble in H₂O, they are soluble in diverse oils and 03 is also soluble in a 3:1 ethanol water ratio. The anionic surfactant 06 is soluble in H₂O but does not create foam.

TABLE E1
properties of arabinonamides

| | SOLUBILITY | SURFACE | CMC | |
|---|---|---|---|---|
| COMPOUND | (H₂O) | TENSION | (%) | FOAM |
| 01 | >6% | 43.5 | no | − |
| 02 | 1.14% | 44.68 | no | − |
| 03 | 3:1 EtOH/H₂O & | − | − | − |
| | in oil | | | |
| 04 | In oil | − | − | − |
| 05 | Yes | − | − | − |
| 06 | Yes | | | +/− |

Although the secondary amide-containing surfactants with L-Ara are not that soluble in H₂O, the D-GalA derivatives have increased solubility due to their negative charge (table E1). 09 has a 6% solubility, 10 and 11 2.5 and 2%, and 12 and 13 less than 1%. The solubility decreases as the carbon chain length increases. Foam appears from an eight carbon chain 11. Although the solubility of 11 is just 2%, it reaches a surface tension of 26 mN/m with a 0.29% concentration. The commercially available c SLS and SLES have a higher surface tension of 29.5-33 mN/M, but with a lower 0.02% concentration. The foam stability of SLES and 11 are also quite similar. Although 11 does not have a large solubility concentration in H₂O, the surface tension and foam stability can be better than that of SLES and SLS.

TABLE E2
properties of galactaric acid amides

| | SOLUBILITY | SURFACE | CMC | |
|---|---|---|---|---|
| COMPOUND | (H₂O) | TENSION | (%) | FOAM |
| 09 | >6% | 50.78 | − | − |
| 10 | ~2.5% | 49.43 | − | − |
| 11 | ~2.0% | 26.0 | 0.17 | ++ |
| 12 | <1% | | | ++ |
| 13 | − | | | − |

Derivatives with an N-alkylation were synthesized. The properties are shown in table E3. It was found that these compounds had increased solubility. It is thought that the extra methyl group on the nitrogen removes the possibility for the surfactants to stack on each other in layers. In this way the compounds 17-22 are better soluble in H₂O and methanol.

TABLE E3
properties of N-alkylated compounds

| | SOLUBILITY | SURFACE | CMC | |
|---|---|---|---|---|
| COMPOUND | (H₂O) | TENSION | (%) | FOAM |
| 17 | Soluble | Not measured | − | ++ |
| 18 | Soluble | Not measured | − | − |
| 19 | Soluble | Not measured | − | ++ |
| 20 | Soluble | Not measured | − | ++ |
| 22 | Soluble | Not measured | − | ++ |

The L-Ara and D-GalA based surfactants obtained via the reductive amination are presented in table E4 and E5, respectively. N-methylated L-Ara compounds 23-25, showed more compatible properties in contrast to reference surfactants 26-28. The surfactants 23-25 create foam, have a lower surface tension, and the solubility is a lot higher for the methyl-hexyl carbon chain 23 with 30%.

TABLE E4
properties of N-alkylated arabinonamides and comparisons

| | SOLUBILITY | SURFACE | CMC | |
|---|---|---|---|---|
| COMPOUND | (H₂O) | TENSION | (%) | FOAM |
| 24 | 0.9% | 28.7 | 0.25% | + |
| | | | | pH > 7 |
| 25 | 0.06% | 26.2 | 0.1% | ++ |
| | | | | pH > 7 |
| 23 | 30% | 31.4 | 2% | + |
| | | | | pH > 7 |
| 29 | 1% | 56.1 | 1% | − |
| 26 | 6.67% | 45.8 | 2% | − |
| 28 | NS | − | − | − |
| 27 | 0.75% | 30.3 | 0.5% | − |

Similar results were obtained for the D-GalA reductive amination surfactants (Table E5). Earlier measurements of compound 24 initially found 0.25% solubility. A significant increase in solubility was reported between 33, 34, 37 and the methylated 30-32, such as 33 is 1% soluble in H₂O and 31 15%, which is a compatible percentage to be used in applications like shampoos. The methylated surfactants create foam in contrast to the non-methylated surfactants. Although 32 is less soluble (1%) because of its longer alkyl chain, it has an enormous stability in foam at the whole pH range and reaches the surface tension of 33.9 mN/m with a critical micelle concentration (CMC) of 0.01%. Moreover, the N-methyl-octyl surfactant 31 showed a good surface tension of 31.5 nM/m, solubility and foam creation.

TABLE E5
properties of N-alkylated galactaric acid amides and comparisons

| | SOLUBILITY | SURFACE | CMC | |
|---|---|---|---|---|
| COMPOUND | (H₂O) | TENSION | (%) | FOAM |
| 30 | 40% | 60.6 | 2% | ++ |
| | | | | PH > 7 |
| 31 | 15% | 32.067 | 1.15% | ++ |
| | | | | pH > 7.3 |
| 32 | 1% | 33.9 | 0.1% | +++ |
| 33 | 1% | 45.1 | 1% | − |
| 35 | 0.9% | | | − |
| 34 | 0.3% | 30.7 | 0.1% | − |
| 37 | 14% | − | − | − |
| | (Poorly in 3:1 | | | |
| | EtOH/H₂O) | | | |

TABLE E5.2
additional properties of N-alkylated galactaric acid amides and comparisons

| | | | | | Foamability (mL) | | | Surface | |
|---|---|---|---|---|---|---|---|---|---|
| | | | | | after (min) | | | tension | CMC |
| Name | Number | Chain | h | Solubility | 1 | 5 | 10 | (nM/m) | (%) |
| rAra6 | 27 | 6 | H | 0.75 | | | | | |
| rAra1.6 | 23 | 6 | Me | 30 | 2 | 0.2 | 0 | 31.4 | 2 |
| rAra8 | 28 | 8 | H | NS | | | | | |
| rAra1.8 | 24 | 8 | Me | 0.9 | 5 | 4.5 | 4 | 28.7 | 0.25 |
| rAra10 | 28B | 10 | H | NS | | | | | |
| rAra1.10 | 24B | 10 | Me | 0.1 | 3 | 3 | 2 | 33.7 | 0.2 |
| rAra12 | 28C | 12 | H | NS | | | | | |
| rAra1.12 | 25 | 12 | Me | 0.06 | 5 | 5 | 4 | 26.2 | 0.1 |
| rGalA6 | 37 | 6 | H | 14 | | | | | |
| rGalA1.6 | 30 | 6 | Me | 40 | 0.2 | 0 | 0 | 42.1 | — |
| rGalA8 | 33 | 8 | H | 1 | | | | | |
| rGalA1.8 | 31 | 8 | Me | 15 | 4.5 | 4 | 4 | 32.1 | 1.15 |
| rGalA10 | 33b | 10 | H | 0.55 | | | | | |
| rGalA1.10 | 25b | 10 | Me | 2 | 6 | 6 | 5 | 32.85 | 0.06 |
| rGalA12 | 34 | 12 | H | 0.3 | | | | | |
| rGalA1.12 | 32 | 12 | Me | 1 | 4.5 | 5 | 3 | 33.9 | 0.1 |
| SLES | | 12 | — | 28 | 4 | 4 | 3 | 33.8 | 0.2 |

It was found that Ara and GalA compounds were generally more soluble than glucose analogues. For instance, rAra1.12 (25) and rGalA1.12 (32) were more soluble than their glucose analogue rGlc1.12 (50), which was found to not be soluble at all in water (solubility 0%).

Amine N-oxide surfactants 41-42 are soluble in H₂O and create foam (Table E6).

TABLE E6
properties N-oxide compounds, Arabinose and Galacturonic Acid in Salt form.

| | NAME | YIELD (%) | SOLUBILITY (H₂O) | SURFACE TENSION | CMC (%) | FOAM |
|---|---|---|---|---|---|---|
| 38 | nAra1.6 | 99 | 19% | 49.275 | 1.60 | − |
| 39 | nAra1.8 | 98 | 11% | 27.734 | 0.55 | ++ |
| 41 | nGalA1.6 | 92 | 17% | 37.727 | 2.58 | − |
| 42 | nGalA1.8 | 81 | 10% | 42.89 | 0.65 | +++ |
| 42B | nGalA1.10 | 36 | 8% | 25.17 | 0.08 | +++ |
| 43 | nGalA1.12 | 73 | 7% | 25.551 | 0.058 | +++ |

A surprising result is the better solubility of compounds based on arabinose but also on galacturonic acid in comparison to those based on glucose. Solubility was measured from glucose N-oxide molecules. Glucose analogues with 10 and 12 carbon chain, nGlc1.10 (45) and nGlc1.12 (46), all had a solubility of <1%. Table E6 reports much higher values.

TABLE E7
properties of commercially available surfactants

| | SOLUBILITY | SURFACE | CMC | |
|---|---|---|---|---|
| COMPOUND | (H₂O) | TENSION | (%) | FOAM |
| SLES | Yes | 36.5 | 0.2 | ++ |
| SCG | Yes | 36.6 | | ++ |
| GLUCOPURE WET | Yes | 27.078 | 0.85 | ++ |
| (C8-C10) | | | | |

TABLE E8
additional properties of compounds and comparisons

| | | | | | Surface | |
|---|---|---|---|---|---|---|
| | Solubility | Foamability | | | Tension | CMC |
| Surfactant | (%) | 1 min | 5 min | 30 min | (nM/m) | (%) |
| nAra1.6 (38) | 19 | 0.5 | 0 | 0 | 53.59 | 1.30 |
| nAra1.8 (39) | 11 | 2 | 2 | 0.5 | 27.73 | 0.55 |
| nGalA1.6 (41) | 17 | 0.5 | 0 | 0 | 42.90 | 2.60 |
| nGalA1.8 (42) | 10 | 6 | 6 | 2 | 42.89 | 0.65 |
| nGalA1.10 (42b) | 8 | 6 | 6 | 5 | 25.17 | 0.08 |
| nGalA1.12 (43) | 7 | 5 | 4 | 4 | 25.55 | 0.058 |
| Glucopure C8/10 | | 7 | 5.5 | 5 | 27.08 | 0.030 |
| Glucopure | <0.1 | 4 | 3 | 3 | 27.79 | 0.014 |
| C12/C14 | | | | | | |
| SLES | 28 | 4 | 4 | 3 | 33.8 | 0.2 |

### Zein Score

The skin irritation potential of the surfactants was investigated using zein solubilisation, which is a commonly known assay. Zein is a very simple mimic of skin protein. The more zein is solubilized by the surfactant the harsher the potential to irritate and dry the human skin. SDS and SLES are commercial surfactants and known to be harsh on the skin. Hence, in this assay SDS was set as “100” to compare with the potentials of other surfactants, which are all executed in duplo. The surfactants were measured at different pH values, a pH of 4-5, 7 and at their original pH as is. SLES showed a skin irritation potential value of 49 at a pH of 7, and an even higher value at pH11, indicating it can be about half as irritating as SDS. In contrast, our bio-based surfactants all showed very low values between 0 and 3. For example, GalA1.8 (31) had a zein score of 1 at a pH 7. This indicates that the synthesized surfactants derived from SBP monosaccharides D-GalA and L-Ara can be mild for the human skin.

Biodegradation of compounds was examined. Biodegradation is important to not pollute the environment or harm plant and animal life. The commercially available surfactant SLES has a primary degradation of 99% in 30 days, which also depends on the initial concentration, using Organization for Economic Co-operation and Development (OECD) 301 procedure, a widely known standardized procedure. This same procedure was used to test rGalA1.8 (31) revealing a biodegradability of 90% in 15 days. The compound was added to an inoculate of activated sludge from a municipal wastewater treatment plant (WWTP), including a blank control (no compound), a positive control (inoculum and sodium acetate), and controls for abiotic degradation (compound in medium), adsorption (compound with sterilized inoculum, boiled before use), and toxicity (inoculum with compound and sodium acetate). The results showed a biodegradation of GalA1.8 faster than the commercial surfactant SLES. Compound GalA1.8 did not inhibit breakdown of acetate. Within 5 days, no nGalA1.12 could be detected in acetate+sludge. No rGalA1.8 could be detected within 15 days. Without acetate, no rGalA1.8 could be detected within 20 days. Compounds showed good CO₂ evolution. Controls showed reliable results.

### 2.3 Conclusions

In conclusion, a green synthesis procedure to synthesized bio-based surfactants was developed. The SBP derived monosaccharides L-Ara and D-GalA were reacted with N-alkylamines via a reductive amination to form a variety of surfactants. In addition, the amines were oxidized resulting in N-oxide surfactants. The optimized conditions for the reduction reaction were a one-pot two-step hydrogenation, with no excess of reagents and ethanol as the solvent. The N-oxidation was suitably performed using H₂O₂. In addition, various properties were determined namely: solubility, foamability, surface tension, emulsion stability, skin irritation, and biodegradability. Interestingly, the methylated bio-based compounds all had an increased solubility in comparison to their non-methylated referents (h=H). Solubility was improved by at least 3 times (compare 32 and 34) up to 15 times (compare 31 and 33) up to 40 times (compare 23 and 27) up to an even higher ratio (compare 30 and 37). Moreover, octyl, decyl and dodecyl alkyl chain-containing surfactants showed a comparable foamability and surface tension with SLES. Also the N-oxides surfactants showed a very interesting result in the better solubility of the new compounds in comparison to Glucose-based analogues. Solubility was measured for glucose N-oxide molecules. Glucose with 10 and 12 carbon chain, nGlc1.10 (45) and nGlc1.12 (46), have a solubility of <1%, compared to values over 7% for new compounds 42B and 43.

These studies indicate the attractive properties of the new molecules derived from SBP: soluble, non-irritating, and significant performance. The surfactants can be used in all types of industries like cosmetics and home care, as cleaning agents, solubilizers, emulsifiers, foaming agents, hydrotropes, and stabilizers, etc. The synthesis of bio-based surfactants with interesting properties is possible by a sustainable hydrogenation and oxidation process to form N-methylalkyl (h=Me) and corresponding N-oxide containing compounds.

1.-10. (canceled)

11. Method for producing an amide-derivative of a saccharide,
comprising the steps of:
i) providing a saccharide;
ii) providing a primary or secondary amine;
iii) reacting the saccharide and the amine in the presence of a metal catalyst at a temperature of at most 60° C. to yield the amide-derivative;
iv) optionally isolating the amide-derivative.

12. The method according to claim 11, wherein the saccharide is derived from biomass,
and/or wherein the saccharide is a monosaccharide.

13. The method according to claim 11, wherein the amine of step ii) is of general formula tail-NH₂, wherein tail is a linear C₃₋₂₀ alkyl.

14. The method according to claim 11, wherein the metal catalyst is a gold catalyst.

15. The method according to claim 11, wherein the reacting of step iii) is performed in the presence of a base.

16. The method according to claim 11, wherein:
a) the reacting of step iii) is performed at a temperature of about 20-60° C.;
b) the metal catalyst is present at about 0.2-15 mol-%;
c) the reacting of step iii) is performed in a protic solvent;
d) the reacting of step iii) is performed in a single step;
e) the reacting of step iii) is performed without the addition of an oxidant; and/or
f) the reacting of step iii) is performed under ambient background radiation.

17. A compound of general formula (I): 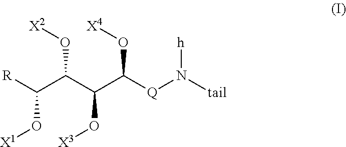
wherein
R is —H, —CH₂—Rr, or —C(═O)—Rr;
Rr is —OH, —Oh¹, —NH₂, —NH(h¹), or —Nh¹h², wherein h¹ and h² are independently a linear, branched, or cyclic C₁₋₆ alkyl, alkenyl, or alkynyl moiety wherein each carbon atom is optionally substituted by halogen, alkoxy, or haloalkoxy;
X¹, X², X³, and X⁴ are in each instance independently hydrogen or a linear, branched, or cyclic C₁₋₆ acyl moiety wherein each carbon atom is optionally substituted by halogen, alkoxy, or haloalkoxy, and wherein the acyl chain is optionally unsaturated, wherein two instances of X¹, X², X³, and X⁴ can together form a bridging moiety to form a five- or six-membered ring;
Q is —CH₂— or —C(═O)—;
h is a hydrogen or a linear, branched, or cyclic C₁₋₂₀ alkyl, alkenyl, or alkynyl moiety wherein each carbon atom is optionally substituted by halogen, alkoxy, or haloalkoxy;
or wherein h is an independently selected instance of tail;
tail is a linear, branched, or cyclic C₁₋₄₀ alkyl, alkenyl, or alkynyl moiety, wherein each carbon atom is optionally substituted by one or more halogen, -h^{t}, —O-h^{t}, —C(═O)Oh^{t}, —C(═O)N(h^{t})₂, —OC(═O)-h^{t}, —N(h^{t})C(═O)-h^{t}, ═O, or —N(h^{t})₂, wherein each instance of h^{t} is independently hydrogen or a linear, branched, or cyclic C₁₋₂₀ alkyl, alkenyl, or alkynyl moiety wherein each carbon atom of h^{t} is optionally substituted by halogen, alkoxy, or haloalkoxy;
or wherein tail is of general formula —(CH₂CH₂O)₁₋₄₅(CH₂)₀₋₂H;
or a salt or N-oxide thereof.

18. Compound according to claim 17, wherein the compound is N-methyl-N-octyl-L-arabinonamide, N-methyl-N-octyl-D-galactaric acid amide, N-methyl-N-hexyl-L-arabinamine, N-methyl-N-octyl-L-arabinamine, N-methyl-N-dodecyl-L-arabinamine, N-methyl-N-hexyl-D-galacturonic acid amine, N-methyl-N-octyl-D-galacturonic acid amine, N-methyl-N-dodecyl-D-galacturonic acid amine, N,N-dioctyl-D-galacturonic acid amine, N-butyl-L-arabinonamide, N-hexyl-L-arabinonamide, N-octyl-L-arabinonamide, N-Decyl-L-arabinonamide, N-isobutyl-L-arabinonamide, N-(decanoic acid)-L-arabinonamide, N-butyl-D-galactaric acid amide, N-hexyl-D-galactaric acid amide, N-octyl-D-galactaric acid amide, N-butyl-L-arabinamine, N-hexyl-L-arabinamine, N-octyl-L-arabinamine, N-(decanoic acid)-L-arabinamine, N-octyl-D-galacturonic acid amine, N-dodecyl-D-galacturonic acid amine, N-(1-methyloctyl)-D-galacturonic acid amine, N-hexyl-D-galacturonic acid amine, N-methyl-N-hexyl-D-galacturonic acid amine oxide, N-methyl-N-octyl-D-galacturonic acid amine oxide, or N-methyl-N-dodecyl-D-galacturonic acid amine oxide, or wherein the compound is N-methyl-N-octyl-L-arabinonamide, N-methyl-N-octyl-D-galactaric acid amide, N-methyl-N-hexyl-L-arabinamine, N-methyl-N-octyl-L-arabinamine, N-methyl-N-dodecyl-L-arabinamine, N-methyl-N-hexyl-D-galacturonic acid amine, N-methyl-N-octyl-D-galacturonic acid amine, N-methyl-N-dodecyl-D-galacturonic acid amine, N,N-dioctyl-D-galacturonic acid amine, N-butyl-L-arabinonamide, N-hexyl-L-arabinonamide, N-octyl-L-arabinonamide, N-Decyl-L-arabinonamide, N-isobutyl-L-arabinonamide, N-(decanoic acid)-L-arabinonamide, N-butyl-D-galactaric acid amide, N-hexyl-D-galactaric acid amide, N-octyl-D-galactaric acid amide, N-methylhexyl-L-arabinonamine oxide, N-methyloctyl-L-arabinonamine oxide, N-methyldecyl-L-arabinonamine oxide, N-methyl-N-hexyl-D-galacturonic acid amine oxide, N-ethyl-N-hexyl-D-galacturonic acid amine oxide, N-methyl-N-octyl-D-galacturonic acid amine oxide, N-methyl-N-octyl-D-galacturonic acid amine oxide, N-methyl-N-dodecyl-D-galacturonic acid amine oxide, 1-(N-methyl-N-octyl)-D-glucosamine oxide, 1-(N-methyl-N-decyl)-D-glucosamine oxide, 1-(N-methyl-N-dodecyl)-D-glucosamine oxide, or 1-(N-methyl-N-hexyl)-D-glucosamine oxide.

19.-20. (canceled)

21. The compound according to claim 17, wherein it is of general formula (I-oxide): 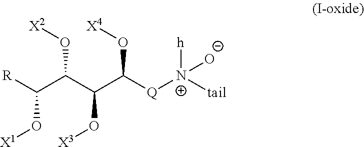
wherein
R is —H, —CH₂—Rr, or —C(═O)—Rr;
Rr is —OH, —Oh¹, —NH₂, —NH(h¹), or —Nh¹h², wherein h¹ and h² are independently a linear, branched, or cyclic C₁₋₆ alkyl, alkenyl, or alkynyl moiety wherein each carbon atom is optionally substituted by halogen, alkoxy, or haloalkoxy;
X¹, X², X³, and X⁴ are in each instance independently hydrogen or a linear, branched, or cyclic C₁₋₆ acyl moiety wherein each carbon atom is optionally substituted by halogen, alkoxy, or haloalkoxy, and wherein the acyl chain is optionally unsaturated, wherein two instances of X¹, X², X³, and X⁴ can together form a bridging moiety to form a five- or six-membered ring;
Q is —CH₂— or —C(═O)—;
h is a hydrogen or a linear, branched, or cyclic C₁₋₂₀ alkyl, alkenyl, or alkynyl moiety wherein each carbon atom is optionally substituted by halogen, alkoxy, or haloalkoxy;
or wherein h is an independently selected instance of tail;
tail is a linear, branched, or cyclic C₁₋₄₀ alkyl, alkenyl, or alkynyl moiety, wherein each carbon atom is optionally substituted by one or more halogen, -h^{t}, —O-h^{t}, —C(═O)Oh^{t}, —C(═O)N(h^{t})₂, —OC(═O)-h^{t}, —N(h^{t})C(═O)-h^{t}, ═O, or —N(h^{t})₂, wherein each instance of h^{t} is independently hydrogen or a linear, branched, or cyclic C₁₋₂₀ alkyl, alkenyl, or alkynyl moiety wherein each carbon atom of h^{t} is optionally substituted by halogen, alkoxy, or haloalkoxy;
or wherein tail is of general formula —(CH₂CH₂O)₁₋₄₅(CH₂)₀₋₂H;
or a salt thereof.

22. The compound according to claim 21, wherein it is of general formula (II-r-oxide):

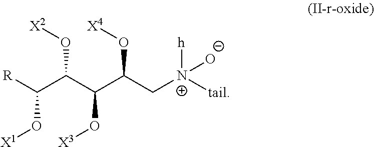

23. The compound according to claim 21, wherein it is of general formula (III-og-oxide), (III-oa-oxide), (III-rg-oxide), or (III-ra-oxide):

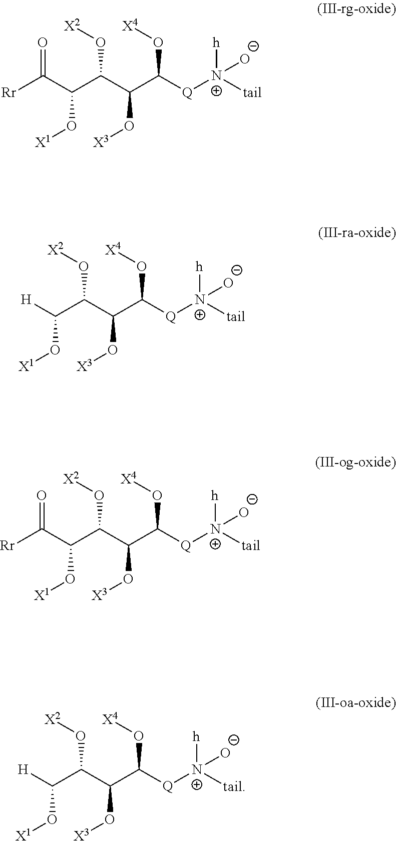

24. The compound according to claim 21, wherein
Rr is —OH or —Oh¹;
h¹ is —CH₃, —CH₂CH₃, —CH(CH₃)₂, or —C(CH₃)₃;
X¹, X², X³, and X⁴ are in each instance independently hydrogen or —C(═O)CH₃, and optionally each of X¹, X², X³, and X⁴ represent the same moiety;
h is hydrogen or a linear, branched, or cyclic C₁₋₂₀ alkyl, alkenyl, or alkynyl moiety;
tail is a linear or branched C₁₋₂₂ alkyl, alkenyl, or alkynyl moiety, wherein up to two carbon atoms are optionally substituted by halogen, -h^{t}, —O-h^{t}, —C(═O)Oh^{t}, C(═O)N(h^{t})₂, —OC(═O)-h^{t}, —N(h^{t})C(═O)-h^{t}, ═O, or —N(h^{t})₂, wherein each instance of h^{t} is independently hydrogen or a linear or branched C₁₋₁₄ alkyl, alkenyl, or alkynyl moiety.

25. The compound according to claim 21, wherein tail is a linear C₃₋₂₀ alkyl.

26. The compound according to claim 21, wherein h is a linear, branched, or cyclic C₁₋₂₀ alkyl, alkenyl, or alkynyl moiety.

27. The compound according to claim 21, wherein it is of general formula (IV-oxide):

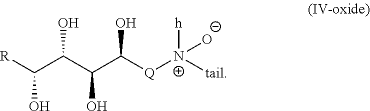

28. The compound according to claim 26, wherein tail is —(CH₂)₃—CH₃, —(CH₂)₅—CH₃, —(CH₂)₇—CH₃, —(CH₂)₉—CH₃, —(CH₂)₁₁—CH₃, —CH₂—CH(CH₃)—CH₃, —(CH₂)₁₀—COOH, —CH(CH₃)—(CH₂)₅—CH₃, —CH(CH₃)—(CH₂)₆—CH₃, —CH₂—CH(CH₂CH₃)—(CH₂)₃—CH₃, —(CH₂CH₂O)₃—CH₂CH₃, or —(CH₂CH₂O)₃—CH₃.

29. The compound according to claim 21, wherein the compound is N-methylhexyl-L-arabinonamine oxide, N-methyloctyl-L-arabinonamine oxide, N-methyldecyl-L-arabinonamine oxide, N-methyl-N-hexyl-D-galacturonic acid amine oxide, N-ethyl-N-hexyl-D-galacturonic acid amine oxide, N-methyl-N-octyl-D-galacturonic acid amine oxide, N-methyl-N-octyl-D-galacturonic acid amine oxide, N-methyl-N-dodecyl-D-galacturonic acid amine oxide, 1-(N-methyl-N-octyl)-D-glucosamine oxide, 1-(N-methyl-N-decyl)-D-glucosamine oxide, 1-(N-methyl-N-dodecyl)-D-glucosamine oxide, or 1-(N-methyl-N-hexyl)-D-glucosamine oxide.

30. Method for producing a compound according to claim 21, the method comprising the steps of:
i) providing a compound of general formula (I): 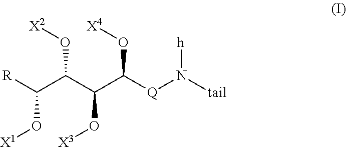
wherein
R is —H, —CH₂—Rr, or —C(═O)—Rr;
Rr is —OH, —Oh¹, —NH₂, —NH(h¹), or —Nh¹h², wherein h¹ and h² are independently a linear, branched, or cyclic C₁₋₆ alkyl, alkenyl, or alkynyl moiety wherein each carbon atom is optionally substituted by halogen, alkoxy, or haloalkoxy;
X¹, X², X³, and X⁴ are in each instance independently hydrogen or a linear, branched, or cyclic C₁₋₆ acyl moiety wherein each carbon atom is optionally substituted by halogen, alkoxy, or haloalkoxy, and wherein the acyl chain is optionally unsaturated, wherein two instances of X¹, X², X³, and X⁴ can together form a bridging moiety to form a five- or six-membered ring;
Q is —CH₂— or —C(═O)—;
h is a linear, branched, or cyclic C₁₋₂₀ alkyl, alkenyl, or alkynyl moiety wherein each carbon atom is optionally substituted by halogen, alkoxy, or haloalkoxy;
or wherein h is an independently selected instance of tail;
tail is a linear, branched, or cyclic C₁₋₄₀ alkyl, alkenyl, or alkynyl moiety, wherein each carbon atom is optionally substituted by one or more halogen, -h^{t}, —O-h^{t}, —C(═O)Oh^{t}, —C(═O)N(h^{t})₂, —OC(═O)-h^{t}, —N(h^{t})C(═O)-h^{t}, ═O, or —N(h^{t})₂, wherein each instance of h^{t} is independently hydrogen or a linear, branched, or cyclic C₁₋₂₀ alkyl, alkenyl, or alkynyl moiety wherein each carbon atom of h^{t} is optionally substituted by halogen, alkoxy, or haloalkoxy;
or wherein tail is of general formula —(CH₂CH₂O)₁₋₄₅(CH₂)₀₋₂H
ii) reacting the provided compound with H₂O₂ to form the N-oxide according to claim 21, and
iii) optionally isolating the N-oxide.

31. The method according to claim 30, wherein h is a linear, branched, or cyclic C₁₋₂₀ alkyl.
